(19) United States
(12) Patent Application Publication
(10) Pub. No.: US 2024/0401157 A1
(43) Pub. Date: Dec. 5, 2024

(54) RESPIRATORY BIOMARKER PANEL

(71) Applicant: Cepheid, Sunnyvale, CA (US)

(72) Inventors: Sara Elfman, Solna (SE); Linda Strömqvist Meuzelaar, Solna (SE)

(73) Assignee: Cepheid, Sunnyvale, CA (US)

(21) Appl. No.: 18/425,806

(22) Filed: Jan. 29, 2024

Related U.S. Application Data
(60) Provisional application No. 63/534,764, filed on Aug. 25, 2023.
(60) Provisional application No. 63/442,070, filed on Jan. 30, 2023.

(51) Int. Cl. C12Q 1/70; B01L 3/00; C12Q 1/689

(52) U.S. Cl. CPC C12Q 1/701; B01L 3/502761; C12Q 1/689; B01L 2200/027; B01L 2200/028; B01L 2200/16; B01L 2300/0681; B01L 2300/1805; C12Q 2600/158

(57) ABSTRACT

The present disclosure provides a set of primers and optional probes for identifying the presence of α-coronavirus, β-coronavirus, SARS-COV-2, adenovirus, Chlamydia pneumoniae, Influenza A, Influenza B, metapneumovirus, rhinovirus/enterovirus, Mycoplasma, Bordetella spp., parainfluenza, and respiratory syncytial virus (RSV), which can be included with (e.g., in at kit) or in a cartridge for automated detection of these pathogens by nucleic acid amplification. This disclosure also provides related detection methods, as well as cartridges, systems, and kits useful in such methods.

### CROSS-REFERENCE TO RELATED APPLICATIONS

This application claims the benefit of U.S. provisional application No. 63/442,070, filed Jan. 30, 2023, and U.S. provisional application No. 63/534,764, filed Aug. 25, 2023, each of which is hereby incorporated by reference in its entirety.

### INCORPORATION BY REFERENCE OF SEQUENCE LISTING PROVIDED AS AN XML FILE

The instant application contains a Sequence Listing which has been submitted electronically in XML file format and is hereby incorporated by reference in its entirety. Said XML copy, created on Apr. 22, 2024, is named CPHDP022US.xml and is 95,728 bytes in size.

### FIELD

The present invention relates to generally to the area of detecting bacterial and/or viral respiratory pathogens.

### BACKGROUND

Respiratory tract infections (RTIs) are among the most common and important problems in clinical medicine around the world. Viruses are responsible for about 90% of upper respiratory tract infections, and about 30% of lower respiratory tract infections. These cause a variety of patient diseases including acute bronchitis, the common cold, influenza, and respiratory distress syndromes. Defining most of these patient diseases is difficult because the presentations connected with RTIs commonly overlap and their causes are similar.

Many of the different types of pathogens that cause RTI led to both upper and lower RTIs that can be local respiratory infections, as well as systemic infections. The most severe cases of RTIs occur in children under the age of 5 years old and in older immunocompromised individuals. The main symptoms of RTIs include coughing, congestion, nasal discharge, wheezing, headache, fever, and myalgia. Symptoms are similar for both bacterial and viral infections making diagnosis difficult without having confirmed diagnostic testing. Without proper diagnosis and treatment of respiratory infections many RTIs can lead to pneumonia. Pneumonia accounts for 16% of all deaths of children under 5 years old and has taken 920,136 children lives in 2015 alone. Pneumonia is the single largest infectious cause of death in children worldwide. Pneumonia is a form of acute respiratory infection in which the small alveoli sacs fill up with pus and fluid, leading to painful breathing and difficulties in oxygen exchange. The presenting symptoms of viral and bacterial pneumonia are similar but require very different treatment regimens. Identifying the pathogen is important for physicians to provide appropriate patient management and treatment.

Respiratory viruses and bacteria have traditionally been detected by cell culture. Viral culture is slow (at least one week of incubation time) and requires a high level of operator experience. Antigen detection methods such as enzyme-linked immunosorbent assay (ELISA) and immunochromatographic (lateral flow) assay and immunofluorescent assay (IFA) are commonly used to detect most respiratory viruses, but these methods suffer from insensitivity. With respect to bacterial respiratory infections, Bordetella species will grow on appropriate agar but more slowly than most other bacteria. For example, it is recommended that cultures be incubated for 10 days to improve recovery. Culture isolation of Bordetella requires prompt transport and specialized medium to maintain the viability of the bacteria. Serologic methods are occasionally used to diagnose pertussis, but there are no FDA-cleared tests, and results can be difficult to interpret, leading to poor sensitivity and specificity. Mycoplasma pneumoniae can be recovered in special culture systems, but requires several weeks of incubation, making it of little use to diagnose of infections. Molecular detection and serologic detection of IgM are the common methods used to diagnose M. pneumoniae infections, and many IgM serology methods suffer from poor specificity. Chlamydia pneumoniae can be recovered in cell culture, but the test is slow, requires expertise, and not available in most laboratories. Serologic methods (IFA) are offered by a few reference laboratories, but results are slow and usually only provide retrospective information.

Molecular methods can provide higher sensitivity and faster time to results than culture. However, current nucleic acid amplification methods have limitations because amplification reaction and signal detection require a controlled environment and precise measurement with expensive instruments. Thus, the methods are often cost-prohibitive for use in point-of-care situations. Additionally, some methods are not optimized for detection of multiplexed target nucleic acids in single patient samples. Detection of multiplexed targets may be accomplished by signal multiplexing in single-pot reactions (fluorescent spectral multiplexing, arrays of electrochemical detectors), physical separation of multiple reactions into unique reaction vessels, or a combination thereof. Physical separation of multiple reactions into unique reaction vessels can lead to erroneous results due to differences in sampling or in assay conditions during reactions, which can confound efforts to make differential diagnoses. Multiplexing can overcome some of these difficulties, but presents its own technical challenges, particularly with attempts to assay for more than a few pathogens in a single reaction mixture. Since the early 2000s, however, DNA-detection technologies have bifurcated into either massively multiplexed but slow systems (next-generation sequencing (NGS) and microarrays), or rapid assays with limited capacity for multiplexing (quantitative PCR (qPCR) and isothermal amplification).

Furthermore, in the case of a CLIA-waived test, no more than three simple steps must be required by the user to simultaneously query a panel of target nucleic acids using a single patient sample. Physical separation of samples into discrete chambers quickly becomes infeasible for CLIA-waived tests, unless a complicated device or disposable automatically handles processing. Spectral multiplexing with fluorescence can reduce the number of unique reactions required to detect a panel of target nucleic acids, but spectral multiplexing LAMP reactions has required dramatic sacrifices in assay speed or signal strength, dampening prospects for successful application to point-of-care testing.

The methods, compositions, and devices presented herein achieve rapid, sensitive, qualitative and optionally quantitative detection of many target nucleic acids (DNA and RNA) from a single sample, in some embodiments, using a closed and affordable instrument.

### SUMMARY

Various embodiments contemplated herein may include, but need not be limited to, one or more of the following:

Embodiment 1: A cartridge for detecting and/or identifying viral and bacterial respiratory pathogens in a sample, the cartridge comprising: a cartridge body comprising a plurality of chambers therein, wherein the plurality of chambers includes: a sample chamber having at least a fluid outlet in fluid communication with another chamber of the plurality; and an optional lysis chamber in fluidic communication with the sample chamber, optionally wherein the sample chamber and lysis chamber are the same; a reaction vessel fluidically coupled to the plurality of chambers of the cartridge body and configured for: i) amplification of nucleic acid and ii) detection and identification of a plurality of amplification products via real-time PCR, melt curve analysis, or a combination thereof; a filter disposed in a fluidic path between the lysis chamber, if present, or the sample chamber, and the reaction vessel, and a set of primers and optional probes for detecting and/or identifying the presence of α-coronavirus, β-coronavirus, SARS-COV-2, adenovirus, Chlamydia pneumoniae, Influenza A, Influenza B, metapneumovirus, rhinovirus/enterovirus, Mycoplasma, Bordetella spp., parainfluenza, and respiratory syncytial virus (RSV).

Embodiment 2: The cartridge of embodiment 1, wherein the lysis chamber comprises one or more lysis reagents for releasing nucleic acid.

Embodiment 3: The cartridge of embodiment 1 or embodiment 2, wherein the sample chamber and the lysis chamber are the same.

Embodiment 4: The cartridge of any one of embodiments 1-3, wherein the reaction vessel comprises one or more reaction chambers for detection of the plurality of amplification products.

Embodiment 5: The cartridge of any one of embodiments 1-4, wherein the reaction vessel comprises one reaction chamber.

Embodiment 6: The cartridge of embodiment 4, wherein each reaction chamber is configured to detect a single amplification product.

Embodiment 7: The cartridge of embodiment 4, wherein each reaction chamber is configured to detect a plurality of amplification products.

Embodiment 8: The cartridge of embodiment 7, wherein each reaction chamber is configured to detect amplification products from α-coronavirus, β-coronavirus, SARS-COV-2, adenovirus, Chlamydia pneumoniae, Influenza A, Influenza B, metapneumovirus, rhinovirus/enterovirus, Mycoplasma, Bordetella spp., parainfluenza, and respiratory syncytial virus (RSV).

Embodiment 9: The cartridge of embodiment 7 or embodiment 8, wherein each reaction chamber is configured to detect and identify simultaneously at least 10 amplification products via real-time PCR.

Embodiment 10: The cartridge of embodiment 7 or embodiment 8, wherein each reaction chamber is configured to detect and identify simultaneously up to 10 amplification products via melt curve analysis.

Embodiment 11: The cartridge of any one of embodiments 1-10, wherein the cartridge is a Clinical Laboratory Improvement Amendments (CLIA)-compliant cartridge.

Embodiment 12: The cartridge of any one of embodiments 1-11, wherein the cartridge is configured to carry out isothermal amplification.

Embodiment 13: The cartridge of any one of embodiments 1-11, wherein the cartridge is configured to carry out non-isothermal amplification, optionally by thermal cycling or temperature oscillation.

Embodiment 14: A method for detecting and/or identifying viral and bacterial respiratory pathogens in a sample, the method comprising: a) contacting nucleic acid from the sample with a set of primers and optional probes for detecting the presence of α-coronavirus, β-coronavirus, SARS-COV-2, adenovirus, Chlamydia pneumoniae, Influenza A, Influenza B, metapneumovirus, rhinovirus/enterovirus, Mycoplasma, Bordetella spp., parainfluenza, and respiratory syncytial virus (RSV); b) subjecting the nucleic acid, primers, and optional probes to amplification conditions followed by a melt curve assay; c) detecting the presence of any amplification product(s) via real-time PCR and melt curve analysis; and d) differentially identifying the presence of a viral and/or bacterial respiratory pathogen in the sample, or determining that no viral or bacterial pathogen detectable using the set of primers is present, based on detection of the amplification product(s) or lack thereof, respectively.

Embodiment 15: The method of embodiment 14, wherein the method further comprises administering a treatment regimen to a subject based on detecting the presence of at least one of the viral or bacterial respiratory pathogens and/or the severity of an infection.

Embodiment 16: The method of embodiment 15, wherein the treatment regimen is selected from one or more of administration of an antibiotics, administration of an anti-viral medication, mechanical ventilation, invasive monitoring, sedation, intensive care admission, surgical intervention, drug of last resort, or hospital admittance.

Embodiment 17: The method of any one of embodiments 14-16, wherein one or more of the bacterial respiratory pathogens are identified via melt curve analysis.

Embodiment 18: The method of any one of embodiments 14-16, wherein the viral and/or bacterial respiratory pathogens identified via melt curve analysis include metapneumovirus, Influenza A, Mycoplasma pneumoniae, Chlamydia pneumoniae, Bordetella parapertussis, and Bordetella pertussis.

Embodiment 19: The method of any one of embodiments 14-18, wherein: a) said contacting nucleic acid from the sample with the set of primers and optional probes comprises: placing the sample in a cartridge comprising a cartridge body having a plurality of chambers in fluidic communication, a reaction vessel having one or more reaction chambers and configured for amplification and detection of the nucleic acid, a fluidic path between the plurality of chambers and the reaction vessel, and a filter in the fluidic path; and if the sample comprises cells, lysing cells in the sample with one or more lysis reagents present within at least one of the plurality of chambers; b) said subjecting the nucleic acid, primer pairs, and optional probes to amplification conditions comprises amplifying the nucleic acid with primers and probes present in solution within the reaction vessel.

Embodiment 20: The method of embodiment 19, wherein amplification is isothermal.

Embodiment 21: The method of embodiment 19, wherein amplification is non-isothermal, preferably by thermal cycling or temperature oscillation.

Embodiment 22: The method of any one of embodiments 19-21, wherein said detecting is carried out in more than one reaction chamber.

Embodiment 23: The method of any one of embodiments 19-21, wherein said detecting is carried out in a single reaction chamber.

Embodiment 24: The method of embodiment 22 or embodiment 23, wherein each reaction chamber detects simultaneously at least 10 amplification products via real-time PCR.

Embodiment 25: The method of any one of embodiments 22-24, wherein each reaction chamber detects simultaneously up to 10 amplification products via melt curve analysis.

Embodiment 26: The method of any one of embodiments 14-25, wherein the sample is a respiratory sample selected from a nasopharyngeal swab (NP), oral-pharyngeal swab (OP), nasal swab (NS), respiratory mucus sample, respiratory tissue sample, respiratory cell sample, saliva sample, sputum sample, or combination thereof.

Embodiment 27: The method of any of embodiments 14-25, wherein the sample is a wastewater sample.

Embodiment 28: The method of any one of embodiments 14-27, wherein said detecting and/or identifying is done at the same facility where the sample was collected from a subject.

Embodiment 29: The method of any one of embodiments 14-28, wherein the method is a point-of-care method.

Embodiment 30: The method of any one of embodiments 14-29, wherein the method is carried out in a hospital, an urgent care center, an emergency room, a physician's office, a health clinic, or a home.

Embodiment 31: The method of any one of embodiments 14-30, wherein the method is a Clinical Laboratory Improvement Amendments (CLIA)-waived test.

Embodiment 32: The method of any one of embodiments 19-30, wherein the cartridge is a Clinical Laboratory Improvement Amendments (CLIA)-compliant cartridge, is operated in compliance with CLIA, is operated by a CLIA-compliant laboratory, or is operated in a CLIA-compliant location.

Embodiment 33: The method of any one of embodiments 19-32, wherein the method is carried out to distinguish between a virulent pathogen and a less virulent pathogen.

Embodiment 34: A kit comprising a cartridge comprising: a set of primers and optional probes for detecting the presence of α-coronavirus, β-coronavirus, SARS-COV-2, adenovirus, Chlamydia pneumoniae, Influenza A, Influenza B, metapneumovirus, rhinovirus/enterovirus, Mycoplasma, Bordetella spp., parainfluenza, RSV, or a combination thereof; and optionally one or more lysis reagents.

Embodiment 35: The cartridge of any one of embodiments 1-13, the method of any one of embodiments 14-33, or the kit of embodiment 34, wherein: the primers and optional probe(s) for detecting α-coronavirus are capable of hybridizing to the spike glycoprotein gene of α-coronavirus; the primers and optional probe(s) for detecting β-coronavirus are capable of hybridizing to the ORF1a gene of β-coronavirus A; the primers and optional probe(s) for detecting SARS-COV-2 coronavirus are capable of hybridizing to the E gene, N gene, RDRP gene, or a combination thereof, of SARS-COV-2 coronavirus; the primers and optional probe(s) for detecting adenovirus are capable of hybridizing to the hexon protein gene of Adenovirus A, B, C, D, and E; the primers and optional probe(s) for detecting Chlamydia are capable of hybridizing to the ompA gene of Chlamydia pneumoniae; the primers and optional probe(s) for detecting Influenza A are capable of hybridizing to the HA gene, the MP gene, the PA gene, the PB gene, or a combination thereof, of Influenza A; the primers and optional probe(s) for detecting Influenza B are capable of hybridizing to the MP gene, NS gene, or a combination thereof of Influenza B; the primers and optional probe(s) for detecting metapneumovirus are capable of hybridizing to the N gene of human metapneumovirus; the primers and optional probe(s) for detecting rhinovirus/enterovirus are capable of hybridizing to 5′ UTR gene of rhinovirus/enterovirus; the primers and optional probe(s) for detecting Mycoplasma are capable of hybridizing to the P1 adhesion protein gene of Mycoplasma; the primers and optional probe(s) for detecting Bordetella comprise primers and optional probe(s) that are capable of hybridizing to the IS1001 gene of Bordetella parapertussis; the primers and optional probe(s) for detecting Bordetella also comprise primers and optional probe(s) that are capable of hybridizing to the IS481 gene of Bordetella pertussis; the primers and optional probe(s) for detecting parainfluenza are capable of hybridizing to the polymerase (L) protein gene, nucleocapsid (NP) protein gene, or a combination thereof, of parainfluenza 1, 2, 3, and 4; and the primers and optional probe(s) for detecting RSV are capable of hybridizing to the RSV A gene, the RSV B gene, or a combination thereof.

Embodiment 36: The cartridge of any one of embodiments 1-13, the method of any one of embodiments 14-33 the kit of embodiment 34, or the cartridge, method, or kit of embodiment 35, wherein the primers and optional probes are configured to detect their target nucleic acid sequences in a single reaction mixture comprising 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 35 distinct primer pairs and optional probes.

Embodiment 37: The cartridge, method, or kit of embodiment 35 or embodiment 36, wherein the primer and/or probe capable of hybridizing to the ORF1a gene of β-coronavirus comprises: at least one primer pair and optional probe specific for the ORF1a gene of CoV-OC43; and/or at least one primer pair and optional probe specific for the ORF1a gene of CoV-HKU1.

Embodiment 38: The cartridge, method, or kit of any one of embodiments 35-37, wherein the set of primers and optional probes capable of hybridizing to the spike glycoprotein gene of α-coronavirus comprises: at least one primer pair and optional probe specific for the S gene of CoV-229E; and/or at least one primer pair and optional probe specific for the S gene of CoV-NL63.

Embodiment 39: The cartridge, method, or kit of any one of embodiments 35-38, wherein, when present: the primers and optional probe(s) capable of selectively hybridizing to the ORF1a gene of CoV-OC43 comprise a sequence that is identical or complementary to at least 15 contiguous nucleotides of SEQ ID NO:24; the primers and optional probe(s) capable of selectively hybridizing to the ORF1a gene of CoV-HKU1 comprise a sequence that is identical or complementary to at least 15 contiguous nucleotides of SEQ ID NO:28; the primers and optional probe(s) capable of selectively hybridizing to the S gene of CoV-229E comprise a sequence that is identical or complementary to at least 15 contiguous nucleotides of SEQ ID NO:20; the primers and optional probe(s) capable of selectively hybridizing to the S gene of CoV-NL63 comprise a sequence that is identical or complementary to at least 15 contiguous nucleotides of SEQ ID NO:32; the primers and optional probe(s) capable of selectively hybridizing to the N gene of SARS-COV-2 comprise a sequence that is identical or complementary to at least 15 contiguous nucleotides of SEQ ID NO:96; the primers and optional probe(s) capable of selectively hybridizing to the RDRP gene of SARS-COV-2 comprise a sequence that is identical or complementary to at least 15 contiguous nucleotides of SEQ ID NO:104; the primers and optional probe(s) capable of selectively hybridizing to the E gene of SARS-COV-2 comprise a sequence that is identical or complementary to at least 15 contiguous nucleotides of SEQ ID NO: 100; the primers and optional probe(s) capable of selectively hybridizing to the hexon protein gene of Adenovirus A, B, C, D, and E comprise a sequence that is identical or complementary to at least 15 contiguous nucleotides of SEQ ID NO:4; the primers and optional probe(s) capable of selectively hybridizing to the ompA gene of Chlamydia pneumoniae comprise a sequence that is identical or complementary to at least 15 contiguous nucleotides of SEQ ID NO: 16; the primers and optional probe(s) capable of selectively hybridizing to the PB gene of Influenza A comprise a sequence that is identical or complementary to at least 15 contiguous nucleotides of SEQ ID NO:40; the primers and optional probe(s) capable of selectively hybridizing to the MP gene of Influenza A comprise a sequence that is identical or complementary to at least 15 contiguous nucleotides of SEQ ID NO:36; the primers and optional probe(s) capable of selectively hybridizing to the PA gene of Influenza A comprise a sequence that is identical or complementary to at least 15 contiguous nucleotides of SEQ ID NO: 44; the primers and optional probe(s) capable of selectively hybridizing to the H1 gene of Influenza A comprise a sequence that is identical or complementary to at least 15 contiguous nucleotides of SEQ ID NO:48; the primers and optional probe(s) capable of selectively hybridizing to the MP gene of Influenza B comprise a sequence that is identical or complementary to at least 15 contiguous nucleotides of SEQ ID NO:52, the primers and optional probe(s) capable of selectively hybridizing to the NS gene of Influenza B comprise a sequence that is identical or complementary to at least 15 contiguous nucleotides of one or more of SEQ ID NO:56; the primers and optional probe(s) capable of selectively hybridizing to the N gene of human metapneumovirus comprise a sequence that is identical or complementary to at least 15 contiguous nucleotides of SEQ ID NO:60; the primers and optional probe(s) capable of selectively hybridizing to 5′ UTR gene of rhinovirus/enterovirus comprise a sequence that is identical or complementary to at least 15 contiguous nucleotides of SEQ ID NO:84; the primers and optional probe(s) capable of selectively hybridizing to the P1 adhesion protein gene of Mycoplasma pneumoniae comprise a sequence that is identical or complementary to at least 15 contiguous nucleotides of SEQ ID NO:64; the primers and optional probe(s) capable of selectively hybridizing to the IS1001 gene of Bordetella parapertussis comprise a sequence that is identical or complementary to at least 15 contiguous nucleotides of SEQ ID NO:8; the primers and optional probe(s) capable of selectively hybridizing to the IS481 gene of Bordetella pertussis comprise a sequence that is identical or complementary to at least 15 contiguous nucleotides of SEQ ID NO: 12; the primers and optional probe(s) capable of selectively hybridizing to the polymerase (L) protein gene of Parainfluenza 1 and 3 comprise a sequence that is identical or complementary to at least 15 contiguous nucleotides of one or more of SEQ ID NOs: 68 and 76, respectively; the primers and optional probe(s) capable of selectively hybridizing to the nucleocapsid (NP) protein gene of Parainfluenza 2 and 4 comprise a sequence that is identical or complementary to at least 15 contiguous nucleotides of one or more of SEQ ID NO: 72 and 80, respectively; the primers and optional probe(s) capable of selectively hybridizing to the RSV A gene comprise a sequence that is identical or complementary to at least 15 contiguous nucleotides of SEQ ID NO:88; and the primers and optional probe(s) capable of selectively hybridizing to the RSV B gene comprise a sequence that is identical or complementary to at least 15 contiguous nucleotides of SEQ ID NO: 92.

Embodiment 40: The cartridge, method, or kit of any one of embodiments 35-38, wherein, when present: the primers and optional probe(s) capable of selectively hybridizing to the ORF1a gene of CoV-OC43 comprise a sequence that is identical or complementary to at least 15 contiguous nucleotides of one or more of SEQ ID NOs: 21, 22, and 23; the primers and optional probe(s) capable of selectively hybridizing to the ORF1a gene of CoV-HKU1 comprise a sequence that is identical or complementary to at least 15 contiguous nucleotides of one or more of SEQ ID NOs: 25, 26, and 27; the primers and optional probe(s) capable of selectively hybridizing to the S gene of CoV-229E comprise a sequence that is identical or complementary to at least 15 contiguous nucleotides of one or more of SEQ ID NOs: 17, 18, and 19; the primers and optional probe(s) capable of selectively hybridizing to the S gene of CoV-NL63 comprise a sequence that is identical or complementary to at least 15 contiguous nucleotides of one or more of SEQ ID NOs: 29, 30, and 31; the primers and optional probe(s) capable of selectively hybridizing to the N gene of SARS-COV-2 comprise a sequence that is identical or complementary to at least 15 contiguous nucleotides of one or more of SEQ ID NOs: 93, 94, and 95; the primers and optional probe(s) capable of selectively hybridizing to the RDRP gene of SARS-COV-2 comprise a sequence that is identical or complementary to at least 15 contiguous nucleotides of one or more of SEQ ID NOs: 101, 102, and 103; the primers and optional probe(s) capable of selectively hybridizing to the E gene of SARS-COV-2 comprise a sequence that is identical or complementary to at least 15 contiguous nucleotides of one or more of SEQ ID NOs: 97, 98, and 99; the primers and optional probe(s) capable of selectively hybridizing to the hexon protein gene of Adenovirus A, B, C, D, and E comprise a sequence that is identical or complementary to at least 15 contiguous nucleotides of one or more of SEQ ID NOs: 1, 2, and 3; the primers and optional probe(s) capable of selectively hybridizing to the ompA gene of Chlamydia pneumoniae comprise a sequence that is identical or complementary to at least 15 contiguous nucleotides of one or more of SEQ ID NOs: 13, 14, and 15; the primers and optional probe(s) capable of selectively hybridizing to the PB gene of Influenza A comprise a sequence that is identical or complementary to at least 15 contiguous nucleotides of one or more of SEQ ID NOs: 37, 38, and 39; the primers and optional probe(s) capable of selectively hybridizing to the MP gene of Influenza A comprise a sequence that is identical or complementary to at least 15 contiguous nucleotides of one or more of SEQ ID NOs: 33, 34, and 35; the primers and optional probe(s) capable of selectively hybridizing to the PA gene of Influenza A comprise a sequence that is identical or complementary to at least 15 contiguous nucleotides of one or more of SEQ ID NOs: 41, 42, and 43; the primers and optional probe(s) capable of selectively hybridizing to the H1 gene of Influenza A comprise a sequence that is identical or complementary to at least 15 contiguous nucleotides of SEQ ID NOs: 45, 46, and 47; the primers and optional probe(s) capable of selectively hybridizing to the MP gene of Influenza B comprise a sequence that is identical or complementary to at least 15 contiguous nucleotides of one or more of SEQ ID NOs: 49, 50, and 51, the primers and optional probe(s) capable of selectively hybridizing to the NS gene of Influenza B comprise a sequence that is identical or complementary to at least 15 contiguous nucleotides of one or more of SEQ ID NOs: 53, 54, and 55; the primers and optional probe(s) capable of selectively hybridizing to the N gene of human metapneumovirus comprise a sequence that is identical or complementary to at least 15 contiguous nucleotides of one or more of SEQ ID NOs: 57, 58, and 59; the primers and optional probe(s) capable of selectively hybridizing to 5′ UTR gene of rhinovirus/enterovirus comprise a sequence that is identical or complementary to at least 15 contiguous nucleotides of one or more of SEQ ID NOs: 81, 82, and 83; the primers and optional probe(s) capable of selectively hybridizing to the P1 adhesion protein gene of Mycoplasma pneumoniae comprise a sequence that is identical or complementary to at least 15 contiguous nucleotides of one or more of SEQ ID NOs: 61, 62, and 63; the primers and optional probe(s) capable of selectively hybridizing to the IS1001 gene of Bordetella parapertussis comprise a sequence that is identical or complementary to at least 15 contiguous nucleotides of one or more of SEQ ID NOs: 5, 6, and 7; the primers and optional probe(s) capable of selectively hybridizing to the IS481 gene of Bordetella pertussis comprise a sequence that is identical or complementary to at least 15 contiguous nucleotides of one or more of SEQ ID NOs: 9, 10, and 11; the primers and optional probe(s) capable of selectively hybridizing to the polymerase (L) protein gene of Parainfluenza 1 and 3 comprise a sequence that is identical or complementary to at least 15 contiguous nucleotides of one or more of SEQ ID NOs: 68 and 76, respectively; the primers and optional probe(s) capable of selectively hybridizing to the nucleocapsid (NP) protein gene of Parainfluenza 2 and 4 comprise a sequence that is identical or complementary to at least 15 contiguous nucleotides of one or more of SEQ ID NO: 72 and 80, respectively; the primers and optional probe(s) capable of selectively hybridizing to the RSV A gene comprise a sequence that is identical or complementary to at least 15 contiguous nucleotides of one or more of SEQ ID NOs: 85, 86, and 87; and the primers and optional probe(s) capable of selectively hybridizing to the RSV B gene comprise a sequence that is identical or complementary to at least 15 contiguous nucleotides of one or more of SEQ ID NO:89, 90, and 91.

Embodiment 41: The cartridge of any one of embodiments 1-13, the method of any one of embodiments 14-33, the kit of embodiment 34, or the cartridge, method, or kit of any one of embodiments 35-40, wherein at least one of the primers and optional probes comprises a detectable label.

Embodiment 42: The cartridge, method, or kit of embodiment 41, wherein at least one probe comprises a fluorescent dye and a quencher molecule.

Embodiment 43: The cartridge of any one of embodiments 1-13, the method of any one of embodiments 14-33, the kit of embodiment 34, or the cartridge, method, or kit of any one of embodiments 35-42, wherein the cartridge or kit further comprises, or the method further employs, a primer pair specific for an exogenous control and/or an endogenous control, wherein the exogenous control is a sample processing control, and wherein the endogenous control is a sample adequacy control.

Embodiment 44: The cartridge of any one of embodiments 1-13, the method of any one of embodiments 19-33, or the kit of embodiment 34, wherein the cartridge or kit facilitates and/or the method comprises detection of a viral and/or bacterial respiratory pathogen within the biological sample within 60 minutes, within 45 minutes, or within 30 minutes of collecting the sample from the subject.

Embodiment 45: The cartridge of any one of embodiments 2-13, the method of any one of embodiments 19-33, the kit of embodiment 34, or the cartridge, method, or kit of any one of embodiments 35-44, wherein the one or more lysis reagents provide simultaneous lysis of viral and bacterial cells.

Embodiment 46: The cartridge of any one of embodiments 2-13, the method of any one of embodiments 19-33, the kit of embodiment 34, or the cartridge, method, or kit of any one of embodiments 35-45, wherein the one or more lysis reagents comprise a chaotropic agent, a chelating agent, a buffer, and a detergent.

Embodiment 47: The cartridge, method, or kit of embodiment 46, wherein the chaotropic agent is selected from guanidinium thiocyanate, guanidinium hydrochloride, alkali perchlorate, alkali iodide, urea, formamide, or a combination thereof.

Embodiment 48: The cartridge of any one of embodiments 2-13, the method of any one of embodiments 19-33, the kit of embodiment 34, or the cartridge, method, or kit of any one of embodiments 35-47, wherein the one or more lysis reagents comprise a guanidinium compound, sodium hydroxide, EDTA, a buffer, and a detergent.

Embodiment 49: The cartridge of any one of embodiments 1-13 or the method of any one of embodiments 19-33, wherein the filter is configured to bind the nucleic acid to be analyzed.

Embodiment 50: The cartridge or method of embodiment 49, wherein the filter comprises glass fibers and optionally a polymeric binder, or the glass fibers are optionally modified with a DNA binding ligand such as an alkylamine, a cycloalkylamine, an alkyloxy amine, a polyamine moiety, an arylamine, an intercalating agent, a DNA groove binder, a peptide, an amino acid, a protein, or a combination thereof.

Embodiment 51: The cartridge or method of embodiment 50, wherein the filter comprises a 500 micron to 2000 microns thick glass fiber disk having a pore size of 0.2 microns to 1 micron.

Embodiment 52: The cartridge of any one of embodiments 1-13 or the method of any one of embodiments 19-33, wherein the filter is configured to bind unwanted material and allow the nucleic acid to pass through.

Embodiment 53: The cartridge of any one of embodiments 1-13, the method of any one of embodiments 19-33, or the kit of embodiment 34, wherein the cartridge further comprises a binding reagent, wash reagent, eluting reagent, or a combination thereof.

Embodiment 54: The cartridge, method, or kit of embodiment 53, wherein the eluting reagent comprises ammonia or an alkali metal hydroxide.

Embodiment 55: The cartridge, method, or kit of embodiment 53 or embodiment 54, wherein the eluting reagent has a pH above about 9, above about 10, or above about 11.

Embodiment 56: The cartridge, method, or kit of any one of embodiments 53-55, wherein the eluting reagent comprises a polyanion, optionally selected from the group consisting of a carrageenan, a carrier nucleic acid, and i-carrageenan with KOH.

Embodiment 57: The cartridge of any one of embodiments 1-13 or the method of any one of embodiments 19-33, wherein the reaction vessel comprises up to 4 reaction chambers.

Embodiment 58: The cartridge of any one of embodiments 1-13 or the method of any one of embodiments 19-33, wherein at least one of the plurality of chambers comprises one or more lyophilized reagents.

Embodiment 59: The cartridge or method of embodiment 58, wherein the one or more lyophilized reagents is/are in the form of one or more beads.

Embodiment 60: The cartridge or method of embodiment 58 or embodiment 59, wherein the one or more lyophilized reagents are selected from primers, probes, a salt, dNTPs, a thermostable polymerase, a reverse transcriptase, or a combination thereof.

Embodiment 61: The cartridge or method of embodiment 60, wherein the one or more lyophilized reagents comprise lyophilized primers and probes.

Embodiment 62: The cartridge of any one of embodiments 1-13 or the method of any one of embodiments 19-33, wherein reagents and components in the reaction vessel are in solution.

Embodiment 63: The cartridge or method of any one of embodiments 1-62, wherein said cartridge or method has a limit of detection (LOD) of greater than or equal to about 25 TCID50/mL for adenovirus, greater than or equal to about 0.1 TCID50/mL for coronavirus 229E, greater than or equal to about 100 copies/mL for coronavirus HKU1, greater than or equal to about 0.1 TCID50/mL for coronavirus NL63, greater than or equal to about 0.1 TCID50/mL for coronavirus OC43, greater than or equal to about 1500 IU/mL for SARS-COV-2, greater than or equal to about 0.5 TCID50/mL for human metapneumovirus (hMPV), greater than or equal to about 4 TCID50/mL for human rhinovirus, greater than or equal to about 8 TCID50/mL for enterovirus, greater than or equal to about 8 TCID50/mL for parainfluenza 1, greater than or equal to about 2 TCID50/mL for parainfluenza 2, greater than or equal to about 10 TCID50/mL for parainfluenza 3, greater than or equal to about 8 TCID50/mL for parainfluenza 4, greater than or equal to 16 TCID50/mL for influenza A H3N2, greater than or equal to about 0.5 TCID50/mL for influenza A H1N1, greater than or equal to about 4.4 TCID50/mL for influenza B, greater than or equal to about 0.1 TCID50/mL for respiratory syncytial virus (RSV) A, greater than or equal to about 0.05 TCID50/mL for respiratory syncytial virus (RSV) B, greater than or equal to about 200 CFU/mL for Bordetella pertussis, greater than or equal to about 8 CFU/mL for Bordetella parapertussis, greater than or equal to about 15 CCU/mL for Mycoplasma pneumoniae, and/or greater than or equal to about 1 TCID50/mL for Chlamydia pneumoniae.

Embodiment 64: A system for detecting pathogens in a biological sample, the system comprising: a module having a receiving bay for receiving the cartridge of any one of embodiments 1-13, wherein the module includes one or more mechanisms within the receiving bay for manipulating a fluid sample within the cartridge, and an instrument that interfaces with the reaction vessel; and a memory having programmable instructions recorded thereon, that are specially configured to operate the module according to a respiratory pathogen assay protocol to determine nucleic acid sequence characteristics of α-coronavirus, β-coronavirus, SARS-COV-2, adenovirus, Chlamydia pneumoniae, Influenza A, Influenza B, metapneumovirus, rhinovirus/enterovirus, Mycoplasma, Bordetella spp., parainfluenza, and respiratory syncytial virus (RSV).

Embodiment 65: The system of embodiment 64, wherein the module and/or system further comprises: a scanner or reader configured to read an identifier on the cartridge; wherein the instructions are configured to determine an applicable protocol based on reading or scanning of the identifier; and wherein the system operates the module according to the applicable protocol based on an input from the scanner or reader.

Embodiment 66: The system of any one of embodiments 64-65, wherein the module and/or system further comprises: an enclosure; a plurality of modules that includes said module, wherein modules are substantially identical and configured to concurrently perform assays on cartridges received therein.

Embodiment 67: The system of any one of embodiments 64-66, further comprising a networking platform for transmitting results derived from module operation.

Embodiment 68: A method for identifying up to forty target nucleic acids in a biological sample collected from a subject, the method comprising: a) providing a sample cartridge comprising: i) a plurality of chambers including: a lysis chamber having at least a fluid outlet in fluid communication with another chamber of the plurality, the lysis chamber adapted for performing mechanical and chemical lysis to release nucleic acid from the biological sample; ii) lyophilized and liquid reagents in two or more of the plurality of chambers, the lyophilized reagents comprising at least one primer set for amplifying and detecting the target nucleic acid(s), and the liquid reagent including a lysis buffer, a binding reagent, and an eluting buffer; and iii) a reaction vessel fluidically coupled to the plurality of chambers of the sample cartridge and configured for performing amplification of the target nucleic acids; b) contacting nucleic acid from the biological sample with the at least one primer set, i) wherein each primer set comprises a forward primer, a reverse primer, and a probe specific for a target nucleic acid in the biological sample, and ii) wherein at least one of the forward primer or the reverse primer within each set comprises one or more modified bases selected from a destabilizing base, a stabilizing base, or a combination thereof; and iii) wherein the probe in each set comprises a detectable moiety for detection via real-time PCR or melt curve analysis; c) subjecting the nucleic acid, primers, and probes within the reaction vessel to amplification conditions, optionally followed by a melt curve assay, d) detecting the presence of any amplification product(s) via real-time PCR and optionally melt curve analysis, by an optical assembly, wherein the optical assembly comprises at least ten optical channels configured to simultaneously transmit excitation beams in at least ten excitation wavelength ranges, and simultaneously detect emitted light in at least ten emission wavelength ranges to and from the reaction vessel, respectively; and c) differentially identifying the presence of the target nucleic acid(s) in the sample, or determining that no target nucleic acid is detectable based on detection of the amplification product(s) or lack thereof, respectively, wherein when greater than 10 target nucleic acids are to be identified, one or more probes for detection via melt curve analysis and one or more probes for detection via real-time PCR are combined in a single optical channel.

Embodiment 69: The method of embodiment 68, wherein the lysis chamber together with the lysis reagent are adapted to lyse viral, bacterial, fungal, protozoan, and epithelial cells present in the biological sample.

Embodiment 70: The method of embodiment 69, wherein the method does not include mechanical lysis of the biological sample.

Embodiment 71: The method of embodiment 69, wherein the method further includes mechanical lysis of the biological sample.

Embodiment 72: The method of any one of embodiments 68-71, wherein the biological sample is selected from a respiratory sample comprising a nasopharyngeal swab (NP) sample, an oral-pharyngeal swab (OP) sample, a nasal swab (NS) sample, a respiratory mucus sample, a respiratory tissue sample, or a respiratory cell sample; a sample comprising saliva, sputum, blood, plasma, serum, semen, spinal fluid, tissue, tear, urine, stool; or a smear preparation, vaginal swab sample, vaginal mucus sample, vaginal tissue sample, vaginal cell sample, bacterial culture, mammalian cell culture, viral culture, human cell sample, bacterial sample, extracellular fluid sample, pancreatic fluid sample, cell lysate, PCR reaction mixture, in vitro nucleic acid modification reaction mixture, or a combination thereof.

Embodiment 73: The method of any one of embodiments 68-72, wherein the target nucleic acids are selected from a viral pathogen, a bacterial pathogen, a protozoan pathogen, or a combination thereof.

Embodiment 74: The method of any one of embodiments 68-73, wherein the target nucleic acids are selected from a viral respiratory pathogen, a bacterial respiratory pathogen, or a combination thereof.

Embodiment 75: The method of any one of embodiments 68-74, wherein the target nucleic acids comprise target nucleic acids from α-coronavirus, β-coronavirus, SARS-CoV-2, adenovirus, Chlamydia pneumoniae, Influenza A, Influenza B, metapneumovirus, rhinovirus/enterovirus, Mycoplasma, Bordetella spp., parainfluenza, respiratory syncytial virus (RSV), or a combination thereof.

Embodiment 76: The method of any one of embodiments 68-75, wherein the target nucleic acids comprise target nucleic acids from α-coronavirus, β-coronavirus, SARS-CoV-2, adenovirus, Chlamydia pneumoniae, Influenza A, Influenza B, metapneumovirus, rhinovirus/enterovirus, Mycoplasma, Bordetella spp., parainfluenza, and respiratory syncytial virus (RSV).

Embodiment 77: The method of any one of embodiments 68-76, wherein the reaction vessel comprises up to 5 reaction chambers, each reaction chamber configured for performing amplification of the up to forty target nucleic acids in solution.

Embodiment 78: The method of embodiment 77, wherein the reaction vessel comprises a single reaction chamber.

Embodiment 79: The method of any one of embodiments 77-78, wherein the optical assembly simultaneously detects up to ten (10) emission wavelength ranges via real-time PCR in each amplification cycle and up to ten (10) emission wavelength ranges in each melt curve cycle, in each reaction chamber.

Embodiment 80: The method of embodiment 79, wherein the method differentially identifies up to thirty (30) target nucleic acids in each melt curve assay, by combining probes for a plurality (up to three) of target nucleic acids in each optical channel and defining melt windows for each target nucleic acid.

Embodiment 81: The method of any one of embodiments 68-80, wherein, when two or more probes are combined in an optical channel, probe(s) detected by melt curve assay are configured to have a Tm below annealing temperature.

Embodiment 82: The method of any one of embodiments 68-81, wherein when greater than ten (10) target nucleic acids are to be identified, target nucleic acids from viruses are detected via real-time PCR, and target nucleic acids from bacteria are detected via melt curve assay.

Embodiment 83: The method of any one of embodiments 68-82, wherein the destabilizing base is selected from a bulky base to reduce non-specific primer-primer interactions, a base that suppresses melting temperature of the primer in double stranded form, a base that suppresses extension of unwanted primer-dimer(s), or a combination thereof.

Embodiment 84: The method of any one of embodiments 68-83, wherein the stabilizing base increases melting temperature of the primer in double-stranded form or reduces secondary structure formation.

Embodiment 85: The method of any one of embodiments 68-84, comprising up to 40 primer sets.

Embodiment 86: The method of any one of embodiments 68-85, comprising at least 10 primer sets.

Embodiment 87: The method of any one of embodiments 68-85, comprising at least 14 primer sets.

Embodiment 88: The method of any one of embodiments 68-87, wherein the primer sets comprise primers and probes according to any one of embodiments 35-40.

Embodiment 89: The method of any one of embodiments 68-88, wherein the cartridge is further adapted for performing high-resolution melt detection, nested PCR, multiphasic detection, or a combination thereof.

Embodiment 90: The method of any one of embodiments 68-89, wherein the method does not include nested PCR.

Embodiment 91: A sample cartridge for identifying up to forty target nucleic acids in a biological sample collected from a subject, the cartridge comprising: a) a cartridge body comprising a plurality of chambers therein, wherein the plurality of chambers includes: i) a sample chamber having at least a fluid outlet in fluid communication with another chamber of the plurality; ii) a lysis chamber in fluidic communication with the sample chamber, wherein the lysis chamber is adapted for performing mechanical and chemical lysis to release nucleic acid from the biological sample, optionally wherein the sample chamber and lysis chamber are the same; i) a lyophilized reagent chamber comprising a lyophilized PCR master mix including at least one primer set for amplifying and detecting the target nucleic acids, further: a) wherein each primer set comprises a forward primer, a reverse primer, and a probe specific for a target nucleic acid in the biological sample, and b) wherein at least one of the forward primer or the reverse primer within each set comprises one or more modified bases selected from a destabilizing base, a stabilizing base, or a combination thereof; and c) wherein the probe in each set comprises a detectable moiety for detection via real-time PCR or melt curve analysis; iii) one or more liquid reagent chambers comprising a lysis buffer, a binding reagent, and an eluting buffer; b) a reaction vessel fluidically coupled to the plurality of chambers of the cartridge body and configured for amplification of nucleic acid and melt curve assay, and adapted for detection of one or a plurality of amplification products via real-time PCR and melt curve analysis by an optical assembly, wherein the optical assembly comprises at least ten optical channels configured to simultaneously transmit excitation beams in at least ten excitation wavelength ranges to the reaction vessel, and simultaneously detect emitted light in at least ten emission wavelength ranges from the reaction vessel; and c) a filter disposed in a fluidic path between the lysis chamber, and the reaction vessel; and wherein when greater than 10 target nucleic acids are to be identified, one or more probes for detection via melt curve analysis and one or more probes for detection via real-time PCR are combined in a single optical channel.

Embodiment 92: A diagnostic assay system for identifying up to forty target nucleic acids in a biological sample collected from a subject, the system comprising: a) a sample cartridge according to embodiment 93; b) a thermal assembly configured to subject the nucleic acid, primers, and probes within the reaction vessel of the sample chamber to amplification conditions, optionally followed by a melt curve assay; c) an optical assembly comprising at least ten optical channels and configured to simultaneously transmit excitation beams in at least ten excitation wavelength ranges, and simultaneously detect emitted light in at least ten emission wavelength ranges to and from the reaction vessel, respectively; d) an electronic device for i) receiving and processing signals detected due to the presence of any amplification product(s) via real-time PCR and melt curve assay, by the optical assembly; ii) differentially identifying the presence of a target nucleic acid in the sample, or determining that no target nucleic acid is detectable based on detection of the amplification product(s), melt curve, or lack thereof; and iii) outputting a real-time PCR result for the sample in the reaction vessel during or after the amplification, and outputting a melting result for the sample in the reaction vessel during or after the melt probe assay, c) a communication interface in communication with an electronic device that comprises a display screen having a user interface that displays one or more graphical elements that permit a user to input a selection corresponding to at least one target assay from a plurality of target assays, which at least one target assay is directed to determining presence, absence or amount of at least one target nucleic acid in the biological sample.

### BRIEF DESCRIPTION OF THE DRAWINGS

FIGS. 1A-1C show an overview of a sample cartridge with a valve assembly configured for performing differing sample processes, including chemical lysing of targets, which is configured for PCR and optional integrated nucleic acid analysis of the respiratory biomarker panel in accordance with some embodiments of the invention. FIG. 1A shows the sample cartridge body with reaction vessel, FIG. 1B shows an exploded view of the sample cartridge, and FIG. 1C shows components of the valve assembly, in accordance with some embodiments.

FIGS. 2A-2D illustrates various valve assemblies A, B, C, D, each suited for one or more types of target lysing, any of which may be used in a respective sample cartridge.

FIGS. 3A-3B show illustrative, but non-limiting, embodiments of the modules and systems (e.g., processing units) for the PCR detection and/or quantification and optional integrated nucleic acid analysis for the biomarker panel. FIG. 3A illustrates a module configured to receive and interact with the valve assembly of the cartridge to operate the cartridge to facilitate sample preparation and analysis. FIG. 3B illustrates a processing unit (e.g., analytical testing unit) of the module that interacts with the fluid sample in the reaction vessel to facilitate sample processing and analytical testing (e.g., PCR and, optionally, nucleic acid analysis) for the biomarker panel.

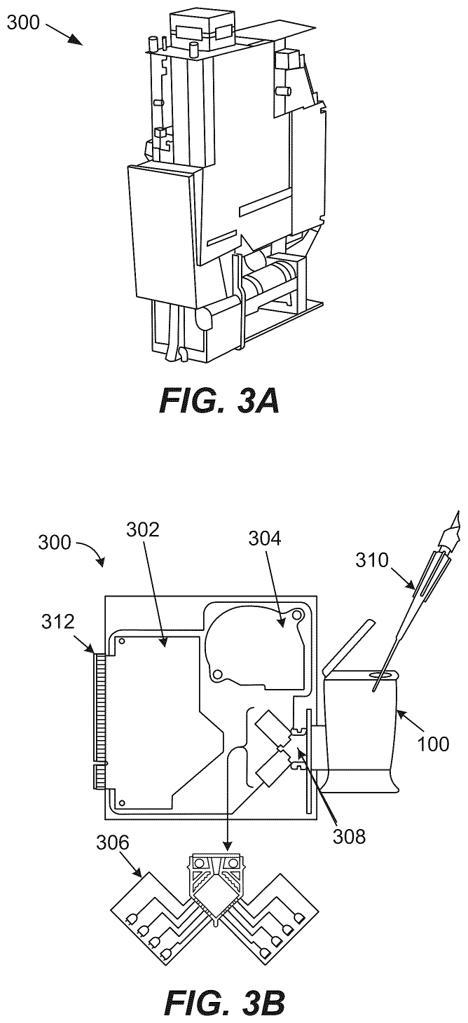

FIG. 4 illustrates an exploded view of the sample cartridge illustrating its major components, including the lid, multi-chamber body, reaction vessel, valve assembly and base, in accordance with some embodiments. The chambers may be used to perform various processes (e.g., extract, purify, deamidate, desulfonate) on the sample.

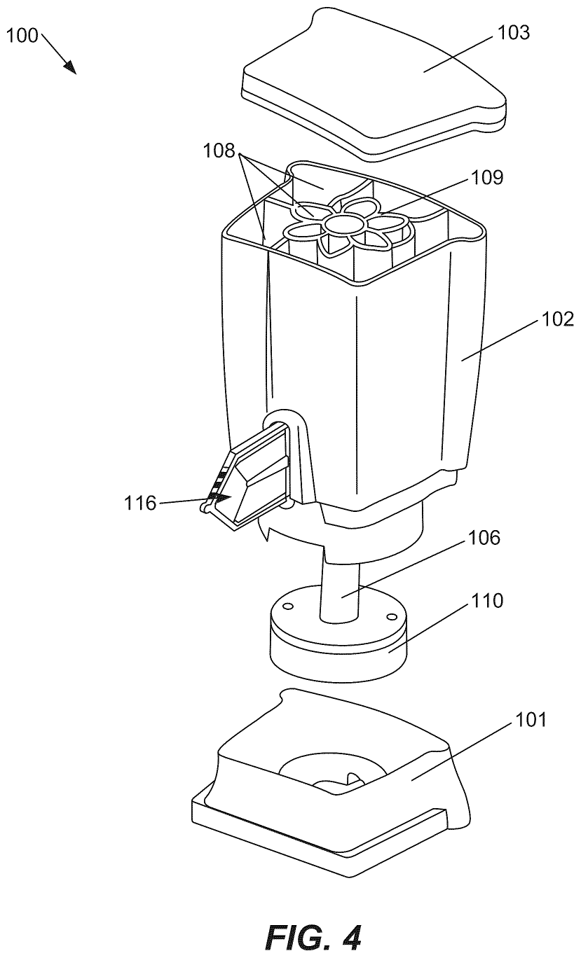

FIGS. 5A-5B show specialized components of the cartridge configured for the targeted assay panel, in accordance with some embodiments. As pictured, a specialized component is a valve assembly having a syringe tube through which the sample is injected and multiple ports on a valve body that facilitate transport of fluid sample between the respective processing chambers upon rotation of the valve assembly.

FIG. 6 shows SARS-COV-2 PQC data from development lot prior to the change. See Example 3.

FIG. 7 shows FLUA PQC data from development lot prior to the change. See Example 3.

FIG. 8 shows RSV PQC data from development lot prior to the change. See Example 3.

FIG. 9 shows RHINO PQC data from development lot prior to the change. See Example 3.

FIG. 10 shows MYCO PQC data from development lot prior to the change. See Example 3.

FIG. 11 shows B. PERTUSSIS PQC data from development lot prior to the change. See Example 3.

### DETAILED DESCRIPTION

The present disclosure describes methods, compositions, devices, and systems that facilitate the rapid detection of viral and bacterial respiratory tract pathogens in a selective and specific manner that is readily automated and can be employed in point-of-care devices, enabling detection of a pathogen responsible for a respiratory tract infection in a subject, so that the appropriate treatment can be administered in a timely manner. The approach relies on nucleic acid amplification to detect selected respiratory pathogens, which entails contacting sample nucleic acids with primers and optional probes that target viral or bacterial nucleic acids for each pathogen, subjecting the nucleic acid, primers, and optional probes to amplification conditions, and detecting the presence of any amplification product(s) to differentially identify the presence of a viral and/or bacterial respiratory pathogen in the sample or to determine that no viral or bacterial pathogen detectable using the set of primers is present in the sample.

### Definitions

Terms used in the claims and specification are defined as set forth below unless otherwise specified.

The term “nucleic acid” refers to a nucleotide polymer, and unless otherwise limited, includes analogs of natural nucleotides that can function in a similar manner (e.g., hybridize) to naturally occurring nucleotides.

The term nucleic acid includes any form of DNA or RNA, including, for example, genomic DNA; complementary DNA (cDNA), which is a DNA representation of mRNA, usually obtained by reverse transcription of messenger RNA (mRNA) or viral RNA or by amplification; DNA molecules produced synthetically or by amplification; mRNA; and non-coding RNA.

The term nucleic acid encompasses double- or triple-stranded nucleic acid complexes, as well as single-stranded molecules. In double- or triple-stranded nucleic acid complexes, the nucleic acid strands need not be coextensive (i.e, a double-stranded nucleic acid need not be double-stranded along the entire length of both strands).

The term nucleic acid also encompasses any modifications thereof, such as by methylation and/or by capping. Nucleic acid modifications can include addition of chemical groups that incorporate additional charge, polarizability, hydrogen bonding, electrostatic interaction, and functionality to the individual nucleic acid bases or to the nucleic acid as a whole. Such modifications may include base modifications such as 2′-position sugar modifications, 5-position pyrimidine modifications, 8-position purine modifications, modifications at cytosine exocyclic amines, substitutions of 5-bromo-uracil, sugar-phosphate backbone modifications, unusual base pairing combinations such as the isobases isocytidine and isoguanidine, and the like.

More particularly, in some embodiments, nucleic acids, can include polydeoxyribonucleotides (containing 2-deoxy-D-ribose), polyribonucleotides (containing D-ribose), and any other type of nucleic acid that is an N- or C-glycoside of a purine or pyrimidine base, as well as other polymers containing nonnucleotidic backbones, for example, polyamide (e.g., peptide nucleic acids (PNAs)) and polymorpholino polymers (see, e.g., Summerton and Weller (1997) “Morpholino Antisense Oligomers: Design, Preparation, and Properties,” Antisense & Nucleic Acid Drug Dev. 7:1817-195; Okamoto et al. (20020) “Development of electrochemically gene-analyzing method using DNA-modified electrodes,” Nucleic Acids Res. Supplement No. 2:171-172), and other synthetic sequence-specific nucleic acid polymers providing that the polymers contain nucleobases in a configuration which allows for base pairing and base stacking, such as is found in DNA and RNA. The term nucleic acid also encompasses locked nucleic acids (LNAs), which are described in U.S. Pat. Nos. 6,794,499, 6,670,461, 6,262,490, and 6,770,748, which are incorporated herein by reference in their entirety for their disclosure of LNAs.

The nucleic acid(s) can be derived from a completely chemical synthesis process, such as a solid phase-mediated chemical synthesis, from a biological source, such as through isolation from any species that produces nucleic acid, or from processes that involve the manipulation of nucleic acids by molecular biology tools, such as DNA replication, PCR amplification, reverse transcription, or from a combination of those processes.

As used herein, the term “gene” encompasses coding sequences, introns, and any associated control sequences that participate in the expression of the coding sequences.

The term “sequence identity,” in the context of two or more amino acid or nucleotide sequences, refers to two or more sequences that are the same or have a specified percentage of amino acid residues or nucleotides that are the same, when compared and aligned for maximum correspondence, as measured using a sequence comparison algorithm or by visual inspection.

For sequence comparison to determine percent nucleotide or amino acid sequence identity, typically one sequence acts as a “reference sequence,” to which a “test” sequence is compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence relative to the reference sequence, based on the designated program parameters. Alignment of sequences for comparison can be conducted using BLAST set to default parameters.

As used herein, the term “complementary” refers to the capacity for precise pairing between two nucleotides; i.e., if a nucleotide at a given position of a nucleic acid is capable of hydrogen bonding with a nucleotide of another nucleic acid to form a canonical base pair, then the two nucleic acids are considered to be complementary to one another at that position. Complementarity between two single-stranded nucleic acid molecules may be “partial,” in which only some of the nucleotides bind, or it may be complete when total complementarity exists between the single-stranded molecules. The degree of complementarity between nucleic acid strands has significant effects on the efficiency and strength of hybridization between nucleic acid strands.

“Selective hybridization” or “selective annealing” refers to the binding of a nucleic acid to a target nucleic acid in the absence of substantial binding to other nucleic acids present in the hybridization mixture under defined stringency conditions. Those of skill in the art recognize that relaxing the stringency of the hybridization conditions allows sequence mismatches to be tolerated.

In some embodiments, hybridizations are carried out under stringent hybridization conditions. The phrase “stringent hybridization conditions” generally refers to a temperature in a range from about 5° C. to about 20° C. or 25° C. below than the melting temperature (Tm) for a specific sequence at a defined ionic strength and pH. As used herein, the Tm is the temperature at which a population of double-stranded nucleic acid molecules becomes half-dissociated into single strands. Methods for calculating the Tm of nucleic acids are well known in the art (see, e.g., Berger and Kimmel (1987) METHODS IN ENZYMOLOGY, VOL. 152: GUIDE TO MOLECULAR CLONING TECHNIQUES, San Diego: Academic Press, Inc. and Sambrook et al. (1989) MOLECULAR CLONING: A LABORATORY MANUAL, 2ND ED., VOLS. 1-3, Cold Spring Harbor Laboratory), both incorporated herein by reference for their descriptions of stringent hybridization conditions). As indicated by standard references, a simple estimate of the Tm value may be calculated by the equation: Tₘ=81.5+0.41 (% G+C), when a nucleic acid is in aqueous solution at 1 M NaCl (see, e.g., Anderson and Young, Quantitative Filter Hybridization in NUCLEIC ACID HYBRIDIZATION (1985)). The melting temperature of a hybrid (and thus the conditions for stringent hybridization) is affected by various factors such as the length and nature (DNA, RNA, base composition) of the primer or probe and nature of the target nucleic acid (DNA, RNA, base composition, present in solution or immobilized, and the like), as well as the concentration of salts and other components (e.g., the presence or absence of formamide, dextran sulfate, polyethylene glycol). The effects of these factors are well known and are discussed in standard references in the art. Illustrative stringent conditions suitable for achieving specific hybridization of most sequences are: a temperature of at least about 60° C. and a salt concentration of about 0.2 molar at pH7. Tₘ calculation for oligonuclotide sequences based on nearest-neighbors thermodynamics can carried out as described in “A unified view of polymer, dumbbell, and oligonucleotide DNA nearest-neighbor thermodynamics” John SantaLucia, Jr., PNAS Feb. 17, 1998 vol. 95 no. 4 1460-1465 (which is incorporated by reference herein for this description).

The term “oligonucleotide” is used to refer to a nucleic acid that is relatively short, generally shorter than 200 nucleotides, more particularly, shorter than 100 nucleotides, most particularly, shorter than 50 nucleotides. Typically, oligonucleotides are single-stranded DNA molecules.

The term “primer” refers to an oligonucleotide that is capable of hybridizing (also termed “annealing”) with a nucleic acid and serving as an initiation site for nucleotide (RNA or DNA) polymerization under appropriate conditions (i.e., in the presence of four different nucleoside triphosphates and an agent for polymerization, such as DNA or RNA polymerase or reverse transcriptase) in an appropriate buffer and at a suitable temperature. The appropriate length of a primer depends on the intended use of the primer, but primers are typically at least 7 nucleotides long and, in some embodiments, range from 10 to 30 nucleotides, or, in some embodiments, from 10 to 60 nucleotides, in length. In some embodiments, primers can be, e.g., 15 to 50 nucleotides long. Short primer molecules generally require cooler temperatures to form sufficiently stable hybrid complexes with the template. A primer need not reflect the exact sequence of the template but must be sufficiently complementary to hybridize with a template.

A primer is said to “anneal to” or “hybridize to” another nucleic acid if the primer, or a portion thereof, hybridizes to a nucleotide sequence within the nucleic acid. The statement that a primer hybridizes to a particular nucleotide sequence is not intended to imply that the primer hybridizes either completely or exclusively to that nucleotide sequence. For example, in some embodiments, amplification primers used herein are said to “anneal to” or be “specific for” a nucleotide sequence.” This description encompasses primers that anneal wholly to the nucleotide sequence, as well as primers that anneal partially to the nucleotide sequence.

The term “primer pair” refers to a set of primers including a 5′ “upstream primer” or “forward primer” that hybridizes with the complement of 5′ end of the DNA sequence to be amplified and a 3′ “downstream primer” or “reverse primer” that hybridizes with 3′ end of the sequence to be amplified. As will be recognized by those of skill in the art, the terms “upstream” and “downstream” or “forward” and “reverse” are not intended to be limiting, but rather provide illustrative orientations in some embodiments.

A “probe” is a nucleic acid capable of binding to a target nucleic acid of complementary sequence through one or more types of chemical bonds, generally through complementary base pairing, usually through hydrogen bond formation, thus forming a duplex structure. The probe can be labeled with a detectable moiety to permit facile detection of the probe, particularly once the probe has hybridized to its complementary target. Alternatively, however, the probe may be unlabeled, but may be detectable by specific binding with a ligand that is labeled, either directly or indirectly. Probes can vary significantly in size.

As used herein with reference to a portion of a primer or a nucleotide sequence within the primer, the term “specific for” a nucleic acid, refers to a primer or nucleotide sequence that can specifically anneal to the target nucleic acid under suitable annealing conditions.

The term “target” is used herein with reference to “target nucleic acids,” as well as “target organisms.” The former refers to nucleic acids to be detected, and the latter refers to organisms to be detected. The term, “target nucleic acid” is generally used herein to refer to a segment of nucleic acid that is defined by a primer pair and that gives rise to an amplicon produced in an amplification reaction; the term “amplification target” is also used herein to refer to this type of target nucleic acid. Primers and probes are also said to “target” nucleic acid sequences, and so these sequences can also be understood as “target nucleic acids.” Additionally, primers and probes are said to “target” or “be specific for” genes. In this usage, the primers and probes can be used to detect the presence of a particular gene by specifically hybridizing to a portion of the gene that indicates its presence. The meaning of “target” and “target nucleic acids” will be clear to one of skill in the art from the context in which the term is employed. In some embodiments, multiple target nucleic acids can be detected to detect a single target organism. In some embodiments, a single target nucleic acid can be detected to detect a single target organism. In some embodiments, an assay can employ multiple target nucleic acids for one or more target organisms and single target nucleic acids for one or more different target organisms.

Amplification according to the present teachings encompasses any means by which at least a part of at least one target nucleic acid is reproduced, typically in a template-dependent manner, including without limitation, a broad range of techniques for amplifying nucleic acid sequences, either linearly or exponentially. Illustrative means for performing an amplifying step include PCR, nucleic acid strand-based amplification (NASBA), two-step multiplexed amplifications, rolling circle amplification (RCA), and the like, including multiplex versions and combinations thereof, for example but not limited to, OLA/PCR, PCR/OLA, LDR/PCR, PCR/PCR/LDR, PCR/LDR, LCR/PCR, PCR/LCR (also known as combined chain reaction—CCR), helicase-dependent amplification (HDA), and the like. Descriptions of such techniques can be found in, among other sources, Ausubel et al.; PCR Primer: A Laboratory Manual, Diffenbach, Ed., Cold Spring Harbor Press (1995); The Electronic Protocol Book, Chang Bioscience (2002); Msuih et al., J. Clin. Micro. 34:501-07 (1996); The Nucleic Acid Protocols Handbook, R. Rapley, ed., Humana Press, Totowa, N.J. (2002); Abramson et al., Curr Opin Biotechnol. 1993 February; 4 (1): 41-7, U.S. Pat. Nos. 6,027,998; 6,605,451, Barany et al., PCT Publication No. WO 97/31256; Wenz et al., PCT Publication No. WO 01/92579; Day et al., Genomics, 29 (1): 152-162 (1995), Ehrlich et al., Science 252:1643-50 (1991); Innis et al., PCR Protocols: A Guide to Methods and Applications, Academic Press (1990); Favis et al., Nature Biotechnology 18:561-64 (2000); and Rabenau et al., Infection 28:97-102 (2000); Belgrader, Barany, and Lubin, Development of a Multiplex Ligation Detection Reaction DNA Typing Assay, Sixth International Symposium on Human Identification, 1995 (available on the world wide web at: promega.com/geneticidproc/ussymp6proc/blegrad.html-); LCR Kit Instruction Manual, Cat. #200520, Rev. #050002, Stratagene, 2002; Barany, Proc. Natl. Acad. Sci. USA 88:188-93 (1991); Bi and Sambrook, Nucl. Acids Res. 25:2924-2951 (1997); Zirvi et al., Nucl. Acid Res. 27: e40i-viii (1999); Dean et al., Proc Natl Acad Sci USA 99:5261-66 (2002); Barany and Gelfand, Gene 109:1-11 (1991); Walker et al., Nucl. Acid Res. 20:1691-96 (1992); Polstra et al., BMC Inf. Dis. 2:18-(2002); Lage et al., Genome Res. 2003 February; 13 (2): 294-307, and Landegren et al., Science 241:1077-80 (1988), Demidov, V., Expert Rev Mol Diagn. 2002 November; 2 (6): 542-8., Cook et al., J Microbiol Methods. 2003 May; 53 (2): 165-74, Schweitzer et al., Curr Opin Biotechnol. 2001 February; 12 (1): 21-7, U.S. Pat. Nos. 5,830,711, 6,027,889, 5,686,243, PCT Publication No. WO0056927A3, and PCT Publication No. WO9803673A1.

In some embodiments, amplification comprises at least one cycle of the sequential procedures of: annealing at least one primer with complementary or substantially complementary sequences in at least one target nucleic acid; synthesizing at least one strand of nucleotides in a template-dependent manner using a polymerase; and denaturing the newly-formed nucleic acid duplex to separate the strands. The cycle may or may not be repeated. Amplification can comprise thermocycling or can be performed isothermally.

As used herein, the term “amplification conditions” refers to conditions that promote amplification of a target nucleic acid in the presence of suitable primers.

As used herein, “in solution” means not immobilized on a substrate of any kind, for example, a bead or a surface in a cassette, such as a chamber wall.

A “multiplex amplification reaction” is one in which two or more nucleic acids distinguishable by sequence are amplified simultaneously.

The term “qPCR” is used herein to refer to quantitative real-time polymerase chain reaction (PCR), which is also known as “real-time PCR” or “kinetic polymerase chain reaction;” all terms refer to PCR with real-time signal detection.

The term “melt curve analysis” refers to the use of the dissociation characteristics of a segment of double-stranded nucleic during heating. Originally, strand dissociation was observed using UV absorbance measurements, but techniques based on fluorescence measurements are now the most common approach. The temperature-dependent dissociation between two DNA-strands can be measured in a “melt assay,” for example, using a DNA-intercalating fluorophore, such as SYBR green or EvaGreen, or fluorophore-labelled DNA probes. In the case of SYBR green (which fluoresces 1000-fold more intensely while intercalated in the minor groove of two strands of DNA), the dissociation of the DNA during heating is measurable by the large reduction in fluorescence that results. Alternatively, juxtapositioned probes (one featuring a fluorophore and the other, a suitable quencher) can be used to determine the complementarity of the probe to the target nucleic acid sequence.

A “reagent” refers broadly to any agent used in a reaction, other than the analyte (e.g., nucleic acid being analyzed). Illustrative reagents for a nucleic acid amplification reaction include, but are not limited to, buffer, metal ions, polymerase, reverse transcriptase, primers, template nucleic acid, nucleotides, labels, dyes, nucleases, dNTPs, and the like. Reagents for enzyme reactions include, for example, substrates, cofactors, buffer, metal ions, inhibitors, and activators.

The term “label,” as used herein, refers to any atom or molecule that can be used to provide a detectable and/or quantifiable signal. In particular, the label can be attached, directly or indirectly, to a nucleic acid or protein. Suitable labels that can be attached to probes include, but are not limited to, radioisotopes, fluorophores, chromophores, mass labels, electron dense particles, magnetic particles, spin labels, molecules that emit chemiluminescence, electrochemically active molecules, enzymes, cofactors, and enzyme substrates.

The term “dye,” as used herein, generally refers to any organic or inorganic molecule that absorbs electromagnetic radiation and produces a detectable signal (e.g., a fluorescent signal).

The term “quencher,” as used herein generally refers to any organic or inorganic molecule that reduces the level of a detectable signal.

As used herein, the term “detecting” refers to “determining the presence of” an item, such as a nucleic acid sequence, e.g., one that is indicative of the presence of a coronavirus. Detection can include the determination of the presence of a coronavirus, without definitive identification of that coronavirus; the determination of the presence of one or more coronaviruses belonging to a class of coronaviruses; the determination of the presence of a particular, known coronavirus strain; or determination of the presence of a novel (not previously described) coronavirus strain.

As used herein, the term “treatment regimen” refers to any medical intervention intended to mitigate the symptoms and/or the pathology of a disorder. The treatment regimen can include one or more actions (e.g., bed rest, increasing fluid intake), non-prescription or prescription medications, supplements, foods, drinks, or the use of medical devices (e.g., a respirator).

As used herein, “Clinical Laboratory Improvement Amendments (CLIA)” refers to The Clinical Laboratory Improvement Amendments of 1988 (CLIA) regulations in effect as of the original filing date of the present application. The CLIA regulations include federal standards applicable to all U.S. facilities or sites that test human specimens for health assessment or to diagnose, prevent, or treat disease. A “CLIA-compliant” test is one that complies with these regulations. “CLIA-waived” tests include tests that does not comply with all of these regulations. For example, CLIA-waived tests include test systems cleared by the U.S. Food and Drug Administration for home use and those tests approved for waiver under the CLIA criteria.

As used herein, the term “virulent” can refer to the degree of infectivity of, and/or the severity of disease induced by, a pathogen, such as a virus. A “more virulent” virus is more infective and/or induces more severe disease than a reference virus and vice versa. In many embodiments, a “less virulent” virus does not typically induce disease requiring hospitalization.

An “endogenous control,” as used herein refers to a moiety that is naturally present in the sample to be used for detection. In some embodiments, an endogenous control is a “sample adequacy control” (SAC), which may be used to determine whether there was sufficient sample used in the assay, or whether the sample comprised sufficient biological material, such as cells. In some embodiments, an endogenous control is an RNA (such as an mRNA, tRNA, ribosomal RNA, etc.), such as a human RNA for a human sample. Nonlimiting exemplary endogenous controls include ABL mRNA, GUSB mRNA, GAPDH mRNA, TUBB mRNA, and UPK1a mRNA. In some embodiments, an endogenous control, such as an SAC, is selected that can be detected in the same manner as the target nucleic acid (e.g., RNA) is detected and, in some embodiments, simultaneously with the target nucleic acid (e.g., RNA).

An “exogenous control,” as used herein, refers to a moiety that is added to a sample or to an assay, such as a “sample processing control” (SPC). In some embodiments, an exogenous control is included with the assay reagents. An exogenous control is typically selected that is not expected to be present in the sample to be used for detection, or is present at very low levels in the sample such that the amount of the moiety naturally present in the sample is either undetectable or is detectable at a much lower level than the amount added to the sample as an exogenous control. In some embodiments, an exogenous control comprises a nucleotide sequence that is not expected to be present in the sample type used for detection of the target nucleic acid (e.g., RNA). In some embodiments, an exogenous control comprises a nucleotide sequence that is not known to be present in the species from whom the sample is taken. In some embodiments, an exogenous control comprises a nucleotide sequence from a different species than the subject from whom the sample was taken. In some embodiments, an exogenous control comprises a nucleotide sequence that is not known to be present in any species. In some embodiments, an exogenous control is selected that can be detected in the same manner as the target nucleic acid (e.g., RNA) is detected and, in some embodiments, simultaneously with the target nucleic acid (e.g., RNA). In some embodiments, the exogenous control is an RNA. In some such embodiments, the exogenous control is an Armored RNA®, which comprises RNA packaged in a bacteriophage protective coat. See, e.g., WalkerPeach et al, Clin. Chem. 45: 12: 2079-2085 (1999).

### Respiratory Pathogens

The present disclosure is aimed at identifying a selection of respiratory pathogens by nucleic acid amplification-based methods. This selection includes α-coronavirus, β-coronavirus, SARS-COV-2, adenovirus, Chlamydia pneumoniae, Influenza A, Influenza B, metapneumovirus, rhinovirus/enterovirus, Mycoplasma, Bordetella spp., parainfluenza, and respiratory syncytial virus (RSV).

### Coronavirus

Coronavirus is an enveloped positive-sense ssRNA virus in the Coronaviridae family. The subfamily Coronavirinae contains four genera: Alpha-, Beta-, Gamma-, and Deltacoronavirus. Alpha- and betacoronaviruses infect mammals, including humans. Their virion contains 4 major structural proteins: the nucleocapsid (N) protein, the transmembrane (M) protein, the envelope (E) protein, and the spike(S) protein. In addition, the CoV genome encodes for 16 non-structural proteins (nsp1-16) that drive genome expression and replication. The CoV genome includes overlapping open reading frames 1a and 1b that express large polyprotein ORF1a and ORF1ab, known as replicase for their demonstrated roles in viral RNA synthesis. ORF1a or ORF1ab are subsequently cleaved by viral proteases.

The seasonal coronaviruses include HKU1 and OC43 (betacoronaviruses), and NL63 and 229E (alphacoronaviruses), which cause mild to moderate upper-respiratory tract infections. They are responsible for episodes of common cold in humans worldwide. Infections are characterized by rhinorrhea, nasal congestion, sore throat, sneezing, and cough with mild fever. The infection may also cause lower-respiratory tract infections, including bronchitis and pneumonia, but it is not as common.

The SARS-COV-2 genome includes genes that code for four structural proteins (S, E, M, and N) and sixteen non-structural proteins (nsp1-16). The nucleocapsid protein (N) forms the capsid outside the genome, and the genome is further packed by an envelope which is associated with three structural proteins: membrane protein (M), spike protein(S), and envelope protein (E). Nsp1 mediates RNA processing and replication. Among non-structural proteins, Nsp12 contains the RNA-dependent RNA polymerase (RdRp), which is a critical composition of coronavirus replication/transcription.

The N2 assay is specific for SARS-COV-2; it does not detect other closely related coronaviruses in the Sarbecovirus subgenus such as SARS-COV-1. This assay was chosen to provide this high level of specificity for SARS-COV-2. The E assay is “Sarbecovirus specific” and will detect not only SARS-COV-2, but other coronaviruses in the Sarbecovirus subgenus. Since SARS-COV-2 is the only member of the Sarbecovirus subgenus known to currently circulated in humans, it is in effect specific for SARS-COV-2. However, should another Sarbecovirus emerge in the human population, the E assay is well poised to detect it. In some embodiments, the assays described herein provide a high level of specificity for SARS-COV-2 by targeting the N2, RdRp and E genes. MERS-COV is a member of the C lineage betacoronaviruses and will not be detected by N2, RdRp and E targets for SARS-COV-2.

### Adenovirus (AdV)

Adenovirus AdV has a 70-100 nm spherical structure and the external shell consists of 252 capsomeres, including 240 hexons and 12 pentons, which are composed of an icosahedral viral capsid. The genome contains early expressed E1-E4 genes that code for replication proteins, intermediate genes of IX and IVa2, and late region genes L1-L5 associated with structural proteins production. Currently, more than 84 genotypes, including all previously characterized serotypes have been identified and grouped into seven different species (A-G), based on their immunochemical responses, nucleic acid characteristics, hexon and fiber protein characteristics, biological properties, and phylogenetic relationships. These viruses can cause infections of respiratory tract (AdV species B, C and E) and can also cause infection in the eyes (conjunctivitis, keratoconjunctivitis), intestinal tract (gastroenteritis), nervous system (meningitis and encephalitis), and urinary tract (AdV species A, D, F and G). The virus can cause upper respiratory tract (URTI) flu-like symptoms; pharyngitis (sore throat), rhinitis (runny nose), cough, ear infection, pinkeye and fever. Treatment is largely directed at the treatment of symptoms with acetaminophen for fever. In severe adenovirus infections cidofovir has been used to treat immunocompromised individuals.

Chlamydia (Chlamydophila) pneumoniae

Chlamydia pneumoniae is an important intracellular pathogen that is responsible for over a million human infections per year. It is an uncommon cause of community-acquired pneumonia and bronchitis. C. pneumoniae infection incubation period is between 3 to 4 weeks and present with a wide spectrum of clinical symptoms. Most patients present with cough, fever, malaise, headache and can develop pharyngitis, laryngitis, coryza, or pneumonia. The ompA gene of C. pneumoniae is highly conserved within the species.

### Influenza Viruses

Influenza viruses belong to the family Orthomyxoviridae and are the leading cause of severe respiratory illness across the world. Influenza virus primarily spreads from one person to another through respiratory droplets from the infected to the healthy person. A typical influenza infection is characterized by fever, chills, myalgias, headache, rhinorrhea, cough, and inflammation of the upper respiratory tract, among others. Appropriate treatment of the patients can be done after accurate and timely diagnosis. An antiviral therapy based on the use of inhibitors of viral proteins is the most common used.

There are four types of influenza viruses: A, B, C and D. Influenza A viruses (IAV) are the only influenza viruses known to cause flu pandemics, whereas both Influenza A and B viruses (IAVs and IBVs) cause seasonal epidemics of disease (known as the flu season) almost every winter. Influenza type C infections, instead, generally cause mild illness and are not thought to cause human flu epidemics, and diagnostic testing for influenza C virus is not routinely performed. Influenza D viruses primarily affect cattle and are not known to infect or cause illness in humans.

The genome of influenza A and B viruses consists of eight single-stranded viral RNA (vRNA) segments. Each segment (named as the corresponding protein) codes for one of the viral proteins, which include the major surface glycoproteins hemagglutinin (HA) and neuraminidase (NA), the nucleocapsid protein (NP), three subunits of the viral RNA-dependent RNA polymerase (RdRP) (PA, PA-X, PB1, PB2, PB1-F2), the matrix proteins (M1, M2) and the nonstructural proteins NS1 and NS2. Influenza A are subtyped according to the antigenicity of the surface glycoproteins into 16 HA- and 9 NA-subtypes.

Importantly, influenza viruses have the ability to rapidly acquire adaptive genetic mutations in a process called antigenic drift, through which they gradually evade the human immune response. Additionally, the segmented genome enables the special re-arrangement of the genetic material of different IAVs strains, giving rise to novel, gene-reassorted virus strains, a process called antigenic shift that periodically results in emergence of virus strains with very altered characteristics and with the ability to infect humans and with an increased pathogenicity during pandemic outbreaks. While Type A viruses undergo both antigenic drift and shift and are the only influenza viruses known to cause pandemics, influenza type B viruses change only by the more gradual process of antigenic drift.

Human Metapneumovirus (hMPV)

Human Metapneumovirus (hMPV) is a negative sense ssRNA virus in the Pneumoviridae family. hMPV is responsible for 5-7% of RTI in children and about 3% among all age groups. Patients symptoms include cough, rhinorrhea, wheezing, dyspnea and fever. During the respiratory virus season hMPV, RSV, and influenza can circulate simultaneously and be difficult to diagnose without confirmatory testing.

The hMPV genome is about 13,000 nucleotides in length and is composed of eight genes encoding for nine proteins: nucleoprotein (N), phosphoprotein (P), matrix protein (M), fusion protein (F), matrix-2 proteins (M2-1 and M2-2), small hydrophobic (SH) protein, glycoprotein (G), and large (L) polymerase protein. The N, L, and P proteins form the viral replication complex. Though similar in genome to RSV, hMPV possesses a gene order different from that of RSV and lacks the non-structural proteins NS1 and NS2.

### Rhinovirus and Enterovirus

Rhinoviruses (RV) and Enteroviruses (EV) are members of the Picornaviridae family. RV and EV are small RNA viruses consisting of a small capsid that contains single-stranded positive-sense RNA genome. The 5′ terminus of the viral RNA includes a non-translated region (NTR) which is well characterized and well conserved among RV and EV, making it a common target for molecular diagnostic tests. The 5′-NTRs of picornaviruses possess a higher G+C content compared to the rest of picornavirus genome, which is important for stability of the secondary structures and adaptation to environment. The 5′-NTR region is characterized by a high degree of sequence homology among picornavirus species.

There are three species of RV-A, B and C, and more than 100 serotypes. All RV serotypes in species A, B and C are capable of causing respiratory symptoms. There are a number of human and non-human species of EV. Human EV species are A, B, C and D. EVs are associated with a number of distinct human diseases, and while many of these can be proceeded by mild upper respiratory symptoms, one EV serotype, D68, is associated with a uniquely respiratory illness.

### Rhinovirus

Unlike EVs, most RVs lose infectivity upon exposure to mild acid, which explains their general inability to infect the gut or to be spread via the fecal-oral route. RVs are the most frequent cause of the common cold (upper respiratory tract infection (URTI)) in all age groups. Complications of URTI include acute otitis media and sinusitis. RVs, in particular, species C, can cause lower respiratory tract disease. Transmission of RV occurs via direct inoculation into nose or eyes, or via respiratory droplets. Illness is often characterized by watery nasal discharge, malaise, nasal congestion, pharyngitis, and cough. Fever is infrequent.

RVs can be identified in most respiratory specimen types, including swabs or aspirates from the nose, pharynx or nasopharynx, as well as sputum, tracheal aspirates and bronchoalveolar lavages (BAL) when lower respiratory tract symptoms are present. RVs in species A and B can be readily cultured in human cell lines. Members of RV species C are not generally culturable. RVs in cell culture can be identified by characteristics of cytopathic effect (CPE) and distinguished from EVs (similar CPE) by their acid lability. This is a cumbersome laboratory test which exposes an aliquot of specimen to an acid solution, followed by a test of culturability. There are no antigen-based tests to detect RVs, nor are there serology tests to detect an immune response that is diagnostic of acute infection. The 5′ NTR of RVs is well conserved among picornaviruses.

### Enterovirus

As previously mentioned, there are four species of human EVs, A-D. EV species A consists of several coxsackievirus A types (e.g. Coxsackievirus A10), and “numbered” enterovirus types (e.g. enterovirus A71). EV species B includes several Coxsackievirus B types, as well as numbered enterovirus types. EV species C includes several Coxsackievirus A types, polio viruses, and numbered enteroviruses. Finally, species D includes several numbered enteroviruses, including enterovirus D68, which is the etiologic agent of severe lower respiratory tract disease in infants. Following infection, initial EV replication occurs in the upper respiratory tract, and then virus may escape the host immune response, disseminate, and infect organ systems such as the central nervous system and heart. EV can be transmitted by both fecal-oral and respiratory routes. Although most EV infections are asymptomatic, millions of symptomatic EV infections are estimated to occur each year in the U.S. Most EV infections cause only mild nonspecific disease, but infections can lead serious illness, especially in infants. EVs are the most common cause of aseptic meningitis, and they all cause exanthems such as hand, foot and mouth disease and herpangina, pericarditis, pleurodynia, myocarditis, acute hemorrhagic conjunctivitis, acute respiratory illness), encephalitis, polio (natural polio is largely eradicated), and acute flaccid myelitis. While some of these diseases may be preceded by a mild common cold syndrome, EV D68 is noteworthy in its association with severe lower respiratory tract disease in infants.

Most EVs can be isolated and identified in cell culture. However, virus isolation is complex, labor-intensive and can take days to weeks to detect cytopathic effect. Given that EVs and RVs can both be isolated from the upper respiratory tract, and cytopathic effect in cell culture can appear identical, a technically-demanding acid lability test (previously described) is required. EV serotype-specific reagents to confirm cytopathic effect are commercially available for relatively few EV serotypes. There are no antigen-based tests or serology methods to diagnose acute infection.

The 5′-NTR is conserved among EVs, as well as RVs (and other picornaviruses, as noted above), making it difficult to use this region to differentiate between the two.

Mycoplasma pneumoniae

Mycoplasma pneumoniae is a bacterium that lacks a cell wall that causes community-acquired atypical pneumonia. There are an estimated 2 million annual cases of M. pneumoniae infections in the U.S. The most common type of illness caused by this bacterial infection is tracheobronchitis. The main symptoms of the infection include being tired and having a sore throat, fever, and productive cough. Attachment to the respiratory epithelium is the first step in the infection process. Adherence of M. pneumoniae to its host cell is a complex event which requires a terminal structure (attachment organelle) at one pole of the bacterial cell which mediates the interaction of the bacterium with the host. In the membrane of the attachment organelle are clustered adhesin proteins that include P1 adhesin.

Bordetella pertussis and B. parapertussis

Bordetella pertussis and B. parapertussis are gram-negative bacteria that are known to cause highly infectious whooping cough. Patients with the infection usually have several weeks of mild cold that progresses to a rapid cough with high-pitched “whoop.” B. parapertussis is known to cause a milder pertussis-like infection.

### Human Parainfluenza Virus (HPIV)

Human Parainfluenza Viruses (HPIVs) are negative-sense, single-stranded, enveloped RNA viruses of the Paramyxoviriade family. There are four different types of HPIVs; HPIV-1, HPIV-2, HPIV-3 and HPIV-4. Parainfluenza virus affects more than 200,000 people in the U.S. population per year. Human Parainfluenza 1 is most common in children (<5 years old). Human Parainfluenza 1 and 2 both cause upper and lower respiratory illness and croup. Human Parainfluenza 3 is more often associated with bronchitis, bronchiolitis, and pneumonia. Human Parainfluenza 4 is less common, but can cause severe pulmonary infections.

The genomes of HPIV1, 2, and 3 are similar in size (15.5-15.7 kb), whereas that of HPIV4 is somewhat larger, however they share the same order of 6 genes. There are two viral surface proteins: the hemagglutinin-neuraminidase (HN) protein, which mediates attachment to host cell membranes and the fusion (F) protein, which mediates the fusion of the viral envelope with the host cell membrane. The N protein coats the genomic RNA, forming a highly stable nucleocapsid. The phosphoprotein (P) and the large polymerase protein (L) are associated with the nucleocapsid, while the matrix protein (M) coats the inner surface of the envelope.

### Respiratory Syncytial Virus (RSV)

RSV is a medium-sized (˜150 nm) enveloped virus. The scientific name for this viral species is Human orthopneumovirus. This is synonymous with Human respiratory syncytial virus (hRSV). It belongs to the Pneumoviridae family. RSV is divided into two antigenic subtypes, A and B, based on the reactivity of F and G surface proteins to monoclonal antibodies. Generally, RSV subtype A (RSVA) is thought to be more virulent than RSV subtype B (RSVB), with higher viral loads and faster transmission time. RSV infection can present with a wide variety of signs and symptoms that range from mild upper respiratory tract infections (URTI) to severe and potentially life-threatening lower respiratory tract infections (LRTI) requiring hospitalization and mechanical ventilation. Treatments include anti-viral therapies, anti-inflammatoires, and bronchodilators.

RSV is a negative-sense, single-stranded RNA virus. The genome is linear and approximately 15,000 nucleotides in length.[9] It is non-segmented which means that, unlike influenza, RSV cannot participate in the type of genetic reassortment and antigenic shifts responsible for large pandemics.[4] It has 10 genes encoding for 11 proteins.[2][4] The gene order is NS1-NS2-N-P-M-SH-G-F-M2-L, with the NS1 and NS2 gene serving as nonstructural promoter genes.

### Respiratory Pathogen Biomarker Targets

In some embodiments, the respiratory pathogens discussed above can be detected by nucleic acid amplification in a respiratory panel assay, for example, in multiplex amplification reactions, which can be designed to detect 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 or more target nucleic acids per amplification reaction mixture. This degree of multiplexing can be achieved by using primers and probes that do not substantially cross-react or bind off-target and that can reliably identify the pathogens they target in the face of antigenic drift. Bioinformatic analysis of multiple data bases can be carried out to identify primers and probes for highly conserved regions in the genomes of these pathogens.

Recognized herein are various issues with currently available multiplexed PCR methods. For instance, while multiplexing a large number of target amplification reactions (e.g., multiplexed PCR) may be possible, it is not straightforward to detect multiple amplicons simultaneously. So far, multiplexed q-PCR methods, defined as the processes by which one amplifies and detects a plurality of nucleic acid sequences simultaneously in a single reaction chamber, has been implemented for a small number of amplicons, generally less than ten. It is of great interest to efficiently multiplex the assays in the same reaction volume and allow for multiple concurrent target amplification and detection in the same reaction chamber. Such an approach may not only better utilize the original DNA sample, but also significantly reduce any complexities associated with the fluidics and liquid-handling procedures for running multiple single-plex reactions.

Attempts at creating multiplexed q-PCR methods have been plagued by practical issues of simultaneously detecting different nucleic acid sequences in a single sample. A possible approach is to associate different reporter molecules (e.g., fluorescent dyes) to individual amplicons during the PCR reaction which may enable parallel detection of individual reporters by different “colors”. While such approach, in theory, may offer parallelism, it is limited by: (i) the number of different reporter molecules available; (ii) crosstalk optical signal present in a channel due to the optical signal in an adjacent channel; and (iii) the availability of imagers and detectors capable of differentiating different signals. Another possible approach to offer multiplexing capability is to divide the biological sample of interest and physically place it, using fluidic systems, into separate, single and isolated amplification chambers. While this approach may effectively create multiplex q-PCR by performing multiple single-plex (i.e., one amplicon per chamber) q-PCR reactions, it may be suboptimal, since it may reduce the number of target nucleic acid sequences in each chamber which may create stochastic anomalies (Poisson noise) in the acquired data when the original sample has a small concentration. Further, it requires complex fluidic handling procedures.

Highly-multiplexed detection of DNA sequences in a sample may be done through adopting analytical platforms such as DNA microarrays or next-generation DNA sequencers, but not q-PCR or equivalent. Microarrays, in particular, are massively-parallel, affinity-based biosensors where target nucleic acids are captured selectively from the same sample at different addressable coordinates (e.g., pixels) on a solid surface. Each addressable coordinate can have a unique capturing DNA or RNA probe, complementary to a target nucleic acid sequence to be detected in the sample. While microarrays may offer high multiplexing capability, they are semiquantitative and are inferior in terms of limit-of-detection (LOD) and detection dynamic range (DDR), due to their end-point detection nature (i.e., no real-time detection) and the fact that they lack any target amplification.

Due to the vast range of targets for primers and probes and target amplicons, a multiplex strategy was followed to screen and select the primers and oligos. The Respiratory Panel multiplex design strategy can involve the following steps: singleplex design for all primer/probes, multiplex with background oligos and matrix for primer/probe, multiplex with primers and probes divided in different pools, sequencing of samples, re-design of primer/probes if required, multiplex with all panel primer/probes together, and repeat one or more steps when required. In this regard, non-specific interactions caused by a high number of oligonucleotides were observed during assay development. Oligonucleotides with the weakest interactions were selected from the sequencing of PCR products and thorough in-silico analysis. The assay was also optimized for salt, enzyme, oligonucleotide concentration and the PCR profile of the over 26 amplicons present in solution.

In addition, to increase the number of target nucleic acids detected per channel, the following approaches were used: (i) Taqman and melt probes were combined in the same channel using melt probe with a Tₘ below annealing temperature (no amplification curve); and (ii) several melt target nucleic acids in one channel; the melt window for each target was dependent on the sequence variation of the target. Several channel options and designs were investigated for each target to find the optimum arrangement. As an example, Rhinovirus and Enterovirus targets are detected in one channel using the same probe. The probe used has more mismatches to the various Enterovirus species (about 70% complementarity) resulting in detection by melt at low melt temperature.

Due to the high mutation rate of the viral target organisms, which made it difficult to find conserved regions, amplification detection was preferred to avoid a large variation in the melt probes and Tₘ windows associated with the mutations.

Because a large number of oligonucleotides combined in one reaction mixture can lead to unwanted interactions between them, in some embodiments, modified nucleotides can be used to reduce primer-primer interactions.

The reference genomes and position of primers and probes for the genes being targeted in an illustrative respiratory panel are provided below in Table A. Particular embodiments of a respiratory biomarker panel may include one amplification target per organism to be detected or more than one amplification target. For example, 1, 2, 3, 4, 5, 6, 7, 8, 9, 10, 11, 12, 13, 14, or all of the organisms listed in Table A can in various embodiments be detected using 2, 3, or more amplification targets per organism.

TABLE A
Respiratory Biomarker Target Nucleic Acids

| | | GenBank | |
|---|---|---|---|
| Organism | Gene | Acc. No. | Position |
| Adenovirus | L3 hexon | KX868289 | 17758-17893 |
| Coronavirus 229E | S (surface glycoprotein) | MN369046 | 22847-22937 |
| Coronavirus NL63 | S (surface glycoprotein) | MN306040 | 23423-23513 |
| Coronavirus OC43 | Replicase polyprotein lab | MN310478 | 16377-16479 |
| Coronavirus HKU1 | Replicase polyprotein lab | MH940245 | 16575-16677 |
| SARS-Cov-2 | E (envelope protein) | NC_045512.2 | 26269-26381 |
| | N2 (nucleocapsid protein) | NC_045512.2 | 29161-29233 |
| | RdRP (RNA-dependent RNA | NC_045512.2 | 15341-15433 |
| | polymerase) | | |
| hMPV | N (nucleoprotein) | MK588637 | 35-197 |
| Rhinovirus/Enterovirus | 5′ UTR | FJ445117 | 440-560 |
| Influenza A | Polymerase PB2 | MN694927 | 2222-2289 |
| | Polymerase PA | MN656033 | 2045-2132 |
| | MP (matrix protein) | MN552954 | 21-165 |
| Influenza A H1N1pdm | HA (hemagglutinin) | MN251005 | 266-365 |
| Influenza B | M (matrix protein 1) | MN061262 | 18-123 |
| | NS (non-structural protein) | MN435076 | 729-840 |
| Parainfluenza 1 | L polymerase | JQ901971 | 10940-11075 |
| Parainfluenza 2 | NP (nucleoprotein) | MG836425 | 1074-1183 |
| Parainfluenza 3 | L polymerase | KJ672531 | 10729-10855 |
| Parainfluenza 4 | Nucleocapsid protein | MH892408 | 332-428 |
| RSV A | N (nucleoprotein) | MG642083 | 1099-1194 |
| RSV B | N (nucleoprotein) | LC495297 | 1126-1218 |
| B. parapertussis | IS1001 | CP043165 | 75193-75111 |
| B. pertussis | IS481 | CP033420 | 28150-28244 |
| C. pneumoniae | ompA (out membrane protein A) | LN849043 | 132804-132906 |
| M. pneumoniae | P1 adhesin | CP017343 | 182786-182888 |

Some embodiments of the respiratory biomarker panel assay can detect one or more targets in one or more additional viral or bacterial pathogens, such as a novel coronavirus, hantavirus, cytomegalovirus, coxsackie virus, herpes simplex virus, echovirus, influenza virus C, Streptococcus pneumoniae, Moraxella catarrhalis, Haemophilus influenzae, Haemophilus parainfluenzae, a group A streptococcus, Streptococcus pyogenes, Klebsiella pneumoniae, a Pseudomonas species, a Neisseria species, Histoplasnia capsulatum, Coccidioides immitis, Blastomyces dermatitidis, Paracoccidioides brasiliensis, a Candida species, an Aspergillus species, a Mucor species, Cryptococcus neoformans, or Pneumocystis carinii.

Nonlimiting exemplary primer pairs and optional probes are shown in Table B.

TABLE B
Primer, Probe, and Amplicon Nucleotide Sequences for
Respiratory Biomarker Targets

| | | | SEQ |
|---|---|---|---|
| | | | ID |
| Target | Oligo Label | SEQUENCE | NO |
| Adenovirus | ADV Fwd | GCC ACG GTG GGG TTT CTA AAC TT | 1 |
| | ADV Rev | TGA TGC CGC AGT GGG CAT ACA TGC A | 2 |
| | ADV Probe | CCC CCC CC TCG GTG CAC CAG ACC CGG GCT CAG GTA CTC CGA GGC | 3 |
| | ADV | GCCACGGTGGGGTTTCTAAACTTGTTTCCCAGGGTGAAGTAGGTGTCCGTGG | 4 |
| | Amplicon | CGCGGGCGAATTGCACCAGACCGGGACTCAGGTACTCCGAGGCATCCTGAC | |
| | | CGGCGATGTGCATGTAAGACCACTGCGGCATCA | |
| Bordetella | PAPT Fwd | CCG GCT CGA CGA ATT G | 5 |
| paraper- | PAPT Rev | CCA GAG CCG TTT GAG TTC GTC | 6 |
| tussis | PAPT Probe | GGC GCC AAC CAG CCG CTG CTG ACG GTC TAT GTC CTG GCG CC | 7 |
| | PAPT | CCGGCTCGACGAATTGCTGCAAGCCAACCAGCCGCTGCTGACGGTCTATGT | 8 |
| | Amplicon | CCTGCGTGACGAACTCAAACGGCTCTGG | |
| Bordetella | PERT Fwd | CAC ACA AAC TTG ATG GGC GAT CAA T | 9 |
| pertussis | PERT Rev | AGC TTG GTC GGA TGA ACA CCC AT | 10 |
| | PERT Probe | CCC CCC GGC GGG TTC GAG TCG ACG TAG GAA GGT CAA TCC CGC C | 11 |
| | PERT | CACACAAACTTGATGGGCGATCAATTGCTGGACCATTTCGAGTCGACGTAGG | 12 |
| | Amplicon | AAGGTCAATCGGGCATGCTTATGGGTGTTCATCCGGCCGGGC | |
| C. | CPNE Fwd | GCG TTG CTT TCC CCT TGT CAA | 13 |
| pneumoniae | CPNE Rev | GAT AGA GAG GCT CCT ACT TGC CAT | 14 |
| | CPNE Probe | CCC CCC AG CCT GCA GAC TTT GTT CTA GTA GCT GTT GTT ACA GGC T | 15 |
| | CPNE | GCGTTGCTTTCCCCTTGCCAACAGACGCTGGCGTAGCAACAGCTACTGGAAC | 16 |
| | Amplicon | AAAGTCTGCGACCATCAATTATCATGAATGGCAAGTAGGAGCCTCTCTATC | |
| Coronavirus | 229E A01 | TAC TGG TGT TAA TGA TGC TAT TAC A | 17 |
| 229E | Fwd | | |
| | 229E Rev | TTG ATT AAC AAC ATC CTG GAT CTT | 18 |
| | 229E Probe | CT TCA CAA GCT CTA CAA AC AGT TGC TAC T | 19 |
| | 229E | TACTGGTGTTAATGATGCTATTACACAAACTTCACAAGCTCTACAAACAGTTG | 20 |
| | Amplicon | CTACTGCACTTAATAAGATCCAGGATGTTGTTAATCAA | |
| Coronavirus | OC43 Fwd | TTT AAT CGT ATA GCT AGT TGT AAA TGG A | 21 |
| OC43 | OC43 Rev | CCT TTT GCG TTT CTG CAG CAA | 22 |
| | OC43 Probe | AG CTA ATG AAT GTA CAG AGC GCT TG | 23 |
| | OC43 | TTTAATCGTATAGCTAGTTGTAAATGGACCGATGTGGATGATTACATACTAGC | 24 |
| | Amplicon | TAATGAATGTACAGAGCGCTTGAAATTGTTTGCTGCAGAAACGCAAAAGG | |
| Coronavirus | HKU1 Fwd | TTT AAT AAG ATA GCT AGT TGT AAA TGG A | 25 |
| HKU1 | HKU1 Rev | CCT TTT GAG TTT CTG CAG CAA | 26 |
| | HKU1 Probe | CT GGC AAA TGA GTG TA TTG AAC GTT TA | 27 |
| | HKU1 | TTTAATAAGATAGCTAGTTGTAAATGGACAGAAGTTGATGATTATGTTCTGGC | 28 |
| | Amplicon | AAATGAGTGTATTGAACGTTTAAAGTTATTTGCTGCAGAAACTCAAAAGG | |
| Coronavirus | NL63 Fwd | TAG TAG CGT TAA TGA TGC TAT TAC A | 29 |
| NL63 | NL63 Rev | TTG ATT AAC AAC ATC CTG AAT CTT | 30 |
| | NL63 Probe | CT GCA GAG GCT ATA CAT ACT GTT ACT ATT GC | 31 |
| | NL63 | TAGTAGCGTTAATGATGCTATTACACAAACTGCAGAGGCTATACATACTGTTA | 32 |
| | Amplicon | CTATTGCACTTAATA AGATTCAGGATGTTGTTAATCAA | |
| Influenza A | FLUA_MP | TTC TAA CCG AGG TCG AAA CG | 33 |
| virus | Fwd | | |
| | FLUA_MP | ATT GGT CTT GTC TTT AGC C | 34 |
| | Rev | | |
| | FLUA_MP | TCA GGC CCC CTC AAA GCC GA | 35 |
| | Probe | | |
| | FLUA_MP | TTCTAACCGAGGTCGAAACGTACGTTCTTTCTATCATCCCGTCAGGCCCCCT | 36 |
| | Amplicon | CAAAGCCGAGATCGCACAGAGACTGGAAAGTGTCTTTGCAGGAAAGAACACA | |
| | | GATCTTGAGGCTCTTATGGAATGGCTAAAGACAAGACCAAT | |
| Influenza A | FLUA_PB | AAA CGG GAC TCT AGC ATA CT | 37 |
| virus | Fwd | | |
| | FLUA_PB | TAA TTG ATG GCC ATC CGA AT | 38 |
| | Rev | | |
| | FLUA_PB | CTG ACA GCC AGA CAG CGA CCA A | 39 |
| | Probe | | |
| | FLUA_PB | AAACGGGACTCTAGCATACTTACTGACAGCCAGACAGCGACCAAAAGAATTC | 40 |
| | Amplicon | GGATGGCCATCAATTA | |
| Influenza A | FLUA_PA | ATC TTG GGG GGC TAT ATG AAG CAA T | 41 |
| virus | Fwd | | |
| | FLUA_PA | AGG AAG GAG TTG AAC CAA G | 42 |
| | Rev | | |
| | FLUA_PA | AAT GAT CCC TGG GTT TTG CT | 43 |
| | Probe | | |
| | FLUA_PA | ATCTTGGGGGGCTATATGAAGCAATCGAGGAGTGCTTGATTAATGATCCCTG | 44 |
| | Amplicon | GGTTTTGCTTAATGCATCTTGGTTCAACTCCTTCCT | |
| Influenza A | FLUA_H1 | CCT GGG AAA TCC AGA GTG TGA ATC | 45 |
| H1N1 2009 | Fwd | | |
| | FLUA_H1 | ATC TCC TGG GTA ACA CGT TCC ATT | 46 |
| | Rev | | |
| | FLUA_H1 | CGG CGT AGA TGT TTC CGC AAT GTA GGG CCA TGA GCT TGA CGC CG | 47 |
| | Probe | | |
| | FLUA_H1 | CCTGGGAAATCCAGAGTGTGAATCACTCTCCACAGCAAGCTCATGGTCCTAC | 48 |
| | Amplicon | ATTGTGGAAACATCTAGTTCAGACAATGGAACGTGTTACCCAGGAGAT | |
| Influenza B | FLUB_MP | TTG GAG ACA CGA TTG CCT A | 49 |
| virus | Fwd | | |
| | FLUB_MP | AGG TCA AAT TCT TTC CCA C | 50 |
| | Rev | | |
| | FLUB_MP | CTG CTA GTT CTG CTT TGC C | 51 |
| | Probe | | |
| | FLUB_MP | TTGGAGACACGATTGCCTACCTGCTTTCATTAACAGAAGATGGAGAAGGCAA | 52 |
| | Amplicon | AGCAGAACTAGCAGAAAAATTACACTGTTGGTTCGGTGGGAAAGAATTTGAC | |
| | | CT | |
| Influenza B | FLUB_NS | GAT GGC CAT CGG ATC CTC A | 53 |
| virus | Fwd | | |
| | FLUB_NS | GCT CTT GAC CAA ATT GGG AT | 54 |
| | Rev | | |
| | FLUB_NS | CAA AGC CAA T(U01)C GAG CAG CTG | 55 |
| | Probe | | |
| | FLUB_NS | GATGGCCATCGGATCCTCAATTCACTCTTCGAGCGTCTTAATGAAGGACATTC | 56 |
| | Amplicon | AAAGCCAATTCGAGCAGCTGAAACTGCGGTGGGAGTCTTATCCCAATTTGGT | |
| | | CAAGAGC | |
| hMPV | HMPV Fwd | CAT ACA AGC ATG CTA TAT TAA AAG AGT CT | 57 |
| | HMPV Rev | CCT ATT TCT GCA GCA TAT TTG TAA T | 58 |
| | HMPV Probe | TTC TTG TTG CAA TGA TGA AGG TGT | 59 |
| | HMPV | CATATAAACATGCTATATTAAAAGAGTCTCAATACACAATAAAAAGAGATGTAG | 60 |
| | Amplicon | GCACCACAACTGCAGTGACACCTTCATCATTGCAGCAAGAGATAACACTTTTG | |
| | | TGTGGAGAGATTCTTTACACTAAACATACTGATTACAAATATGCTGCAG | |
| | | AGATAGG | |
| M. | MPNE Fwd | CGC ACC CTA CTA CTT CCA CAA T | 61 |
| pneumoniae | MPNE Rev | ATC CTT ACC GTT CTT GTC CTC TC | 62 |
| | MPNE1 | ATA AAG GTA TCA ACT ACA GTT TG | 63 |
| | Probe | | |
| | MPNE | CGCACCCTACTACTTCCACAATAACCCCGATTGACAAGACCGTCCAATCCAA | 64 |
| | Amplicon | AATGTGGTTGATGCCTTTATTAAGCCCTGAGAGGACAAGAACGGTAAGGAT | |
| Parain- | PIV1 Fwd | CAT AAT CCA AGA GGA GGA ATA G | 65 |
| fluenza 1 | PIV1 Rev | CTA TGG CTT GAT TGT CTC CTT GAA CCATT | 66 |
| | PIV1 Probe | CAG CTG CAA GAT GGA TT GCA CTA ATA GAG AT | 67 |
| | PIV1 | CATAATCCAAGAGGAGGAATAGAAGGTTATTGCCAGAAATTATGGACACTAAT | 68 |
| | Amplicon | CTCTATTAGTGCAATCCATCTTGCAGCTGTTAAAGTTGGTGTCAGAGTGTCAG | |
| | | CAATGGTACAAGGAGACAATCAAGCTATAG | |
| Parain- | PIV2 Fwd | TAG GAC CCA TGG CCA AGT ACA T | 69 |
| fluenza 2 | PIV2 Rev | GAC AGT TCC AAT ACC CAT TGC AT | 70 |
| | PIV2 Probe | CAC CAA AGC TGA TGG ATT TTG TCC CAT C | 71 |
| | PIV2 | TAGGACCCATGGCCAAGTACATGGCTCTATTAGAATCACCAAAGCTGATGGA | 72 |
| | Amplicon | TTTTGTCCCATCTGAATATCCATTAGTCTATAGTTATGCAATGGGTATTGGAAC | |
| | | TGTC | |
| Parain- | PIV3 Fwd | CAT AAC CCA AGA GGG GGT ATA G | 73 |
| fluenza 3 | PIV3 Rev | CTATGGCTTGATTGTCTCCTTGAACCATT | 74 |
| | PIV3 Probe | CAG CTG CTA GAT GTA TTG CAC TTATG GAT A | 75 |
| | PIV3 | CATAACCCAAGAGGGGGTATAGAAGGATTTTGTCAAAAATTATGGACACTCAT | 76 |
| | Amplicon | ATCTATAAGTGCAATACATCTAGCAGCTGTTAGAATAGGCGTGAGGGTGACT | |
| | | GCAATGGTTCAAGGAGACAATC | |
| Parain- | PIV4 Fwd | TTT CCT TGC AGG CCA CAT CAA TGC AG | 77 |
| fluenza 4 | PIV4 Rev | GGG TCT ATT GCA TCA ACT TCA ATG | 78 |
| | PIV4 Probe | GCA TCT GGG GCT CTG GC AGC AAT C | 79 |
| | PIV4 | TTTCCTTGCAGGCCACATCAATGCAGAATCATCTTATGATTGCTGCCAGAGCC | 80 |
| | Amplicon | CCAGAT GCTGCTCTTAGGATTATTGAAGTTGATGCAATAGATCC | |
| Rhinovirus/ | HRV Fwd1 | GTC CTC CGG TCC CTG AAT | 81 |
| Enterovirus | HRV Rev | GAA ACA CGG ACA CCC AAA G | 82 |
| | HRV Probe | CCC CCC GT CGT AAT GGG TAA TTG TGG GAT GGG ACC GAC T | 83 |
| | HRV | GTCCTCCGGCCCCTGAATGCGGCTAACCCTAACCCTGCAGCCATTGCCCAC | 84 |
| | Amplicon | AATCCAGTGGGTTTATGGTCGTAATGAGCAATTGCGGGATGGGACCGACTAC | |
| | | TTTGGGTGTCCGTGTTTC | |
| Respiratory | RSVA_Fwd | TAC ACT CAA CAA AGA TCA ACT TCT GT | 85 |
| syncytial | RSVA Rev | TTC TGC ACA TCA TAA TTA GGA GTG TCA | 86 |
| virus | RSVA Probe | CTC CTG TGC TCC GTT GGA TGG T | 87 |
| | RSVA | TACACTCAACAAAGATCAACTTCTGTCATCCAGCAAATACACCATCCAACGGA | 88 |
| | Amplicon | GCACAGGAGATAGTATTGACACTCCCAATTATGATGTGCAGAA | |
| Respiratory | RSVB_Fwd | CAT TAA ATA AGG ATC AGC TGC TGT | 89 |
| syncytial | RSVB Rev | TTT GCA CAT CAT AAT TGG GAG TGT CA | 90 |
| virus | RSVB Probe | TCT CCT GTA CTA CGT TGA ATA GTG T | 91 |
| | RSVB | CATTAAATAAGGATCAGCTGCTGTCATCCAGCAAATACACTATTCAACGTAGT | 92 |
| | Amplicon | ACAGGAGATAATATTGACACTCCCAATTATGATGTGCAAA | |
| SARS- | COV_N2 | TGA TTA CAA ACA TTG GCC GCA A | 93 |
| CoV-2 | Fwd | | |
| | COV_N2 | AAA GCG CGA CAT TCC GAA GA | 94 |
| | Rev | | |
| | COV_N2 | CTG AAG CGC TGG GGG CAA ATT GT | 95 |
| | Probe | | |
| | COV_N2 | TGATTACAAACATTGGCCGCAAATTGCACAATTTGCCCCCAGCGCTTCAGCG | 96 |
| | Amplicon | TTCTTCGGAATGTCGCGCATT | |
| SARS- | COV_E Fwd | ACA GGT ACG TTA ATA GTT AAT AGC GT | 97 |
| CoV-2 | COV_E Rev | ATA TTG CAG CAG TAC GCA CAC | 98 |
| | COV_E | ACA CTA GCC ATC CTT AC TGC GCT TCG | 99 |
| | Probe | | |
| | COV_E | ACAGGTACGTTAATAGTTAATAGCGTACTTCTTTTTCTTGCTTTCGTGGTATTC | 100 |
| | Amplicon | TTGCTAGTTACACTAGCCATCCTTACTGCGCTTCGATTGTGTGCGTACTGCTG | |
| | | CAATAT | |
| SARS- | COV_RDRP | CCT CAC TTG TTC TTG CTC GCA A | 101 |
| CoV-2 | Fwd | | |
| | COV_RDRP | CAC TCA ATA CTT GAG CAC ACT CAT | 102 |
| | Rev | | |
| | COV_RDRP | CAA CGT GTT GTA GCT TGT CAC ACC | 103 |
| | Probe | | |
| | COV_RDRP | CCTCACTTGTTCTTGCTCGCAAACATACAACGTGTTGTAGCTTGTCACACCGT | 104 |
| | Amplicon | TTCTATAGATTAGCTAATGAGTGTGCTCAAGTATTGAGTG | |

The considerations for primers and optional probes for detecting respiratory pathogen biomarkers are described in more detail below in the section entitled “Exemplary Polynucleotides.”

An illustrative respiratory biomarker panel assay that uses the primers and probes of Table B includes 80 oligonucleotides, total. Fifty-three of these are primers, of which 38 are “simple” primers that can include 0, 1, 2 or 3 bulky groups, and 15 are modified primers comprising 1 or more bulky groups and 1 or more chemically modified bases. Such bulky groups and chemically modified bases can provide increased stability of primer-target complex as compared to unmodified primers as well as a barrier to mis-amplification (such as reduction in primer-dimer formation and block certain primer extensions). The assay also includes 27 probes, with 22 being single-dye probes and 5 being double-dye probes. More information on polynucleotide modifications is provided in the section below entitled “Polynucleotide Modifications.” More information on dyes and other labels useful in this assay is provide in the section below entitled “Polynucleotide Labels.

### Controls

In some embodiments, an assay described herein comprises detecting the respiratory pathogen biomarkers described above and at least one endogenous control. In some embodiments, the endogenous control is a sample adequacy control (SAC). In some such embodiments, if no respiratory pathogen biomarker is detected in a sample, and the SAC is also not detected in the sample, the assay result is considered “invalid” because the sample may have been insufficient. While not intending to be bound by any particular theory, an insufficient sample may be too dilute, contain too little cellular material, or contain an assay inhibitor, etc. In some embodiments, the failure to detect an SAC may indicate that the assay reaction failed. In some embodiments, an endogenous control is an RNA (such as an mRNA, tRNA, ribosomal RNA, etc.). Nonlimiting exemplary endogenous controls include ABL mRNA, GUSB mRNA, GAPDH mRNA, TUBB mRNA, and UPK1a mRNA.

In some embodiments, an assay described herein comprises detecting the respiratory pathogen biomarkers described above and at least one exogenous control. In some embodiments, the exogenous control is a sample processing control (SPC). In some such embodiments, if no respiratory pathogen biomarker described above is detected in a sample, and the SPC is also not detected in the sample, the assay result is considered “invalid” because there may have been an error in sample processing, including but not limited to, failure of the assay. Nonlimiting exemplary errors in sample processing include, inadequate sample processing, the presence of an assay inhibitor, the presence of a nuclease (such as an RNase), or compromised reagents, etc. In some embodiments, an exogenous control (such as an SPC) is added to a sample. In some embodiments, an exogenous control (such as an SPC) is added during performance of an assay, such as with one or more buffers or reagents. In some embodiments, when a GeneXpert® system is to be used, the SPC is included in the GeneXpert® cartridge. In some embodiments, an exogenous control (such as an SPC) is an Armored RNA®, which is protected by a bacteriophage coat.

In some embodiments, an endogenous control and/or an exogenous control is/are detected contemporaneously, such as in the same assay, as detection of the respiratory pathogen biomarkers. In some embodiments, an assay comprises reagents for detecting the respiratory pathogen biomarkers described above, and a SAC and/or an exogenous control, simultaneously in the same assay reaction mixture. In some such embodiments, for example, an assay reaction mixture comprises primer sets for amplifying the respiratory pathogen biomarkers described above, a primer set for amplifying a SAC and/or a primer set for amplifying an exogenous control, as well as optional labeled probes for detecting the amplification products (such as, for example, TaqMan® probes).

### Polynucleotides

In some embodiments, polynucleotides are provided for detecting the biomarkers described above. In some embodiments, synthetic polynucleotides are provided. Synthetic polynucleotides, as used herein, refer to polynucleotides that have been synthesized in vitro either chemically or enzymatically. Chemical synthesis of polynucleotides includes, but is not limited to, synthesis using polynucleotide synthesizers, such as OligoPilot™ (GE Healthcare), ABI 3900 DNA Synthesizer (Applied Biosystems), and the like. Enzymatic synthesis includes, but is not limited, to producing polynucleotides by enzymatic amplification, e.g., PCR. A polynucleotide may comprise one or more analog of the canonical nucleotides (e.g., modified nucleotides).

In some embodiments, a polynucleotide is provided that comprises a region that is at least 85%, at least 90%, at least 91%, at least 92%, at least 93%, at least 94%, at least 95%, at least 96%, at least 97%, at least 98%, at least 99%, or 100% identical to, or at least 85%, at least 90%, at least 91%, at least 92%, at least 93%, at least 94%, at least 95%, at least 96%, at least 97%, at least 98%, at least 99%, or 100% complementary to, at least 6, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 contiguous nucleotides of the respiratory pathogen targets, and/or exemplary controls discussed above.

In various embodiments, an exemplary polynucleotide comprises at least: 5, 6, 7, 8, 9, 10, 11, 12, 13, 14, 15, 16, 17, 18, 19, 20, 21, 22, 23, 24, 25, 26, 27, 28, 29, or 30 nucleotides. In various embodiments, a polynucleotide comprises fewer than: 200, 150, 100, 50, 40, 30, or 20 nucleotides. In various embodiments, an exemplary polynucleotide is between 6 and 200, between 8 and 200, between 8 and 150, between 8 and 100, between 8 and 75, between 8 and 50, between 8 and 40, between 8 and 30, between 15 and 100, between 15 and 75, between 15 and 50, between 15 and 40, or between 15 and 30 nucleotides long.

In some embodiments, detection of each target nucleic acid can be carried out using a single labeled primer or probe, specific for each target nucleic acid. Different primers and/or probes can have the same label. By using primers or probes labeled with different detectable moieties (e.g., different fluorescent reporter dyes), numerous target nucleic acids can be detected simultaneously in a single reaction mixture. In some embodiments, 2, 3, 4, 5, 6, 7, 8, 9, 10, 11, 12, 13, 14, 15, 16, 17, 18, 19, or 20 or more different labels can be used in a single reaction mixture or a plurality of reaction mixtures. Each target nucleic acid can be independently monitored using such multiplexing technology. In some embodiments, detection of a plurality of target nucleic acids can be carried out using a single labeled primer or probe. A melt curve may be generated in order to distinguish two or more target nucleic acids that each use the same label, but such analysis may not be necessarily required.

### Polynucleotide Modifications

In some embodiments, the methods of detecting at least one target nucleic acid described herein employ one or more polynucleotides that have been modified, such as polynucleotides comprising one or more affinity-enhancing nucleotide analogs. Modified polynucleotides useful in the methods described herein include primers for reverse transcription, PCR amplification primers, and probes. In some embodiments, the incorporation of affinity-enhancing nucleotides increases the binding affinity and specificity of a polynucleotide for its target nucleic acid as compared to polynucleotides that contain only the canonical deoxyribonucleotides, which allows for the use of shorter polynucleotides or for shorter regions of complementarity between the polynucleotide and the target nucleic acid.

In some embodiments, affinity-enhancing nucleotide analogs include nucleotides comprising one or more base modifications, sugar modifications, and/or backbone modifications. In some embodiments, modified bases for use in affinity-enhancing nucleotide analogs include 5-methylcytosine, isocytosine, pseudoisocytosine, 5-bromouracil, 5-propynyluracil, 6-aminopurine, 2-aminopurine, inosine, diaminopurine, 2-chloro-6-aminopurine, xanthine and hypoxanthine. In some embodiments, affinity-enhancing nucleotide analogs include nucleotides having modified sugars such as 2′-substituted sugars, such as 2′-O-alkyl-ribose sugars, 2′-amino-deoxyribose sugars, 2′-fluoro-deoxyribose sugars, 2′-fluoro-arabinose sugars, and 2′-O-methoxyethyl-ribose (2′MOE) sugars. In some embodiments, modified sugars are arabinose sugars, or d-arabino-hexitol sugars.

In some embodiments, affinity-enhancing nucleotide analogs include backbone modifications such as the use of peptide nucleic acids (PNA; e.g., an oligomer including nucleobases linked together by an amino acid backbone). Other backbone modifications include phosphorothioate linkages, phosphodiester-modified nucleic acids, combinations of phosphodiester and phosphorothioate nucleic acid, methylphosphonate, alkylphosphonates, phosphate esters, alkylphosphonothioates, phosphoramidates, carbamates, carbonates, phosphate triesters, acetamidates, carboxymethyl esters, methylphosphorothioate, phosphorodithioate, p-ethoxy modifications, and combinations thereof.

In some embodiments, a polynucleotide includes at least one affinity-enhancing nucleotide analog that has a modified base, at least nucleotide (which may be the same nucleotide) that has a modified sugar, and/or at least one internucleotide linkage that is non-naturally occurring.

In some embodiments, an affinity-enhancing nucleotide analog contains a locked nucleic acid (“LNA”) sugar, which is a bicyclic sugar. In some embodiments, a polynucleotide for use in the methods described herein comprises one or more nucleotides having an LNA sugar. In some embodiments, a polynucleotide contains one or more regions consisting of nucleotides with LNA sugars. In other embodiments, a polynucleotide contains nucleotides with LNA sugars interspersed with deoxyribonucleotides. See, e.g., Frieden, M. et al. (2008) Curr. Pharm. Des. 14 (11): 1138-1142.

### Primers

In some embodiments, the polynucleotide is a primer. Primers useful in the methods described herein are generally capable of selectively hybridizing to: genomic DNA, a target RNA (genomic or transcript), a cDNA reverse transcribed from the target RNA, and/or an amplicon that has been amplified from genomic DNA, a target RNA, or a cDNA (collectively referred to as “template”), and, in the presence of the template, a polymerase and suitable buffers and reagents, can be extended to form a primer extension product. Primers are generally of a sufficient length to ensure selective hybridization to their target nucleic acids. Generally, primers of at least 15 nucleotides in length hybridize specifically in most contexts, and this length can be reduced, e.g., by including of affinity-enhancing modifications, such as those discussed above. Primers can but need not be exactly complementary to their target nucleic acids. Primers can have any degree of complementarity described above for exemplary polynucleotides. In illustrative embodiments, primers can be 8 to 40 nucleotides in length and at least 90% complementary to their target nucleic acids; 8 to 40 nucleotides in length and at least 95% complementary to their target nucleic acids; 8 to 40 nucleotides in length and at least 99% complementary to their target nucleic acids; 8 to 30 nucleotides in length and at least 90% complementary to their target nucleic acids; 8 to 30 nucleotides in length and at least 95% complementary to their target nucleic acids; 8 to 30 nucleotides in length and at least 99% complementary to their target nucleic acids. In embodiments wherein a primer is less than 100% complementary to it target nucleic acid, having 3′ nucleotide in the primer be complementary to its target nucleic acid facilitates the production of an extension product.

In some embodiments, a primer that selectively hybridizes to its target nucleic acid hybridizes to its target nucleic acid with at least 5-fold greater affinity than to non-target nucleic acid under the same assay conditions. In some embodiments, a primer that selectively hybridizes to its target nucleic acid hybridizes to its target nucleic acid with at least 10-fold greater affinity than to non-target nucleic acid under the same assay conditions.

In some embodiments, a primer pair is designed to produce an amplicon that is 50 to 1500 nucleotides long, 50 to 1000 nucleotides long, 50 to 750 nucleotides long, 50 to 500 nucleotides long, 50 to 400 nucleotides long, 50 to 300 nucleotides long, 50 to 200 nucleotides long, 50 to 150 nucleotides long, 100 to 300 nucleotides long, 100 to 200 nucleotides long, or 100 to 150 nucleotides long.

In some embodiments, the primer is labeled with a detectable moiety. In some embodiments, a primer is not labeled.

### Probes

In some embodiments, the polynucleotide is a probe. Probes useful in the methods described herein are generally capable of selectively hybridizing to: genomic DNA, a target RNA (genomic or transcript), a cDNA reverse transcribed from the target RNA, and/or an amplicon that has been amplified from genomic DNA, a target RNA, or a cDNA (collectively referred to as “template”). Generally, probes of at least 15 nucleotides in length hybridize specifically in most contexts, and this length can be reduced, e.g., by including of affinity-enhancing modifications, such as those discussed above. Probes can but need not be exactly complementary to their target nucleic acids. For example, probes can deliberately include “mismatches” to adjust the Tm of a melt probe. Probes can have any degree of complementarity described above for exemplary polynucleotides. In illustrative embodiments, probes can be 8 to 40 nucleotides in length and at least 70% complementary to their target nucleic acids; 8 to 40 nucleotides in length and at least 75% complementary to their target nucleic acids; 8 to 40 nucleotides in length and at least 80% complementary to their target nucleic acids; 8 to 40 nucleotides in length and at least 85% complementary to their target nucleic acids; 8 to 40 nucleotides in length and at least 90% complementary to their target nucleic acids; 8 to 40 nucleotides in length and at least 95% complementary to their target nucleic acids; 8 to 40 nucleotides in length and at least 99% complementary to their target nucleic acids; 8 to 30 nucleotides in length and at least 90% complementary to their target nucleic acids; 8 to 30 nucleotides in length and at least 95% complementary to their target nucleic acids; 8 to 30 nucleotides in length and at least 99% complementary to their target nucleic acids. In embodiments wherein a probe is less than 100% complementary to a target nucleic acid, any points or regions of non-complementarity are typically located so as not to disrupt the ability of the probe to selectively hybridize to its target nucleic acid.

In some embodiments, a probe that selectively hybridizes to its target nucleic acid hybridizes to its target nucleic acid with at least 5-fold greater affinity than to non-target nucleic acid under the same assay conditions. In some embodiments, a probe that selectively hybridizes to its target nucleic acid hybridizes to its target nucleic acid with at least 10-fold greater affinity than to non-target nucleic acid under the same assay conditions.

### Polynucleotide Labels

In some embodiments, the primer or probe is labeled with a detectable moiety. Detectable moieties include directly detectable moieties, such as fluorescent dyes, and indirectly detectable moieties, such as members of binding pairs. When the detectable moiety is a member of a binding pair, in some embodiments, the probe can be detectable by incubating the probe with a detectable label bound to the second member of the binding pair. In some embodiments, a primer or probe is not labeled, such as when a primer or probe is immobilized, e.g., on a microarray or bead. A labeled primer is extendable, e.g., by a polymerase. In some embodiments, a probe is extendable. In other embodiments, a probe is not extendable. The following discussion centers on probes, as these are more typically employed for detecting in the methods described here, but those of skill in the art appreciate that the polynucleotide labeling strategies described below apply equally to the labeling of primers.

In some embodiments, the probe is a FRET probe that, in some embodiments, is labeled at 5′-end with a fluorescent dye (donor) and at 3′-end with a quencher (acceptor), a chemical group that absorbs (i.e., suppresses) fluorescence emission from the dye when the groups are in close proximity (e.g., attached to the same probe). Thus, in some embodiments, the emission spectrum of the dye should overlap considerably with the absorption spectrum of the quencher. In other embodiments, the dye and quencher are not at the ends of the FRET probe.

Illustrative FRET probes, which include, but are not limited to, a TaqMan® probe, a Molecular beacon probe and a Scorpion probe. A TaqMan® probe is a linear probe that typically has a fluorescent dye covalently bound at one end of the DNA and a quencher molecule covalently bound elsewhere, such as at the other end of the DNA. The FRET probe comprises a sequence that is complementary to a region of the cDNA or amplicon such that, when the FRET probe is hybridized to the cDNA or amplicon, the dye fluorescence is increased due to increased distance between dye and quencher; when the FRET probe is non-hybridized, the dye fluorescence is quenched; and when the probe is digested during amplification of the cDNA or amplicon, the dye is released from the probe and produces a fluorescence signal. In some embodiments, the amount of target nucleic in the sample is proportional to the amount of fluorescence measured during amplification.

Like TaqMan® probes, Molecular Beacons use FRET to detect a PCR product via a probe having a fluorescent dye and a quencher attached at the ends of the probe. Unlike TaqMan® probes, Molecular Beacons remain intact during the PCR cycles. Molecular Beacon probes form a stem-loop structure when free in solution, thereby allowing the dye and quencher to be in close enough proximity to cause fluorescence quenching. When the Molecular Beacon hybridizes to a target nucleic acid, the stem-loop structure is abolished so that the dye and the quencher become separated in space and the dye fluoresces. Molecular Beacons are available, e.g., from Gene Link™ (see www.genclink.com/newsite/products/mbintro.asp).

In some embodiments, Scorpion probes can be used as sequence-specific primers and for PCR product detection. Like Molecular Beacons, Scorpion probes form a stem-loop structure when not hybridized to a target nucleic acid. However, unlike Molecular Beacons, a Scorpion probe achieves both sequence-specific priming and PCR product detection. A fluorescent dye molecule is attached to 5′-end of the Scorpion probe, and a quencher is attached elsewhere, such as to 3′-end. The 3′ portion of the probe is complementary to the extension product of the PCR primer, and this complementary portion is linked to the 5′-end of the probe by a non-amplifiable moiety. After the Scorpion primer is extended, the target-specific sequence of the probe binds to its complement within the extended amplicon, thus opening up the stem-loop structure and allowing the dye on the 5′-end to fluoresce and generate a signal. Scorpion probes are available from, e.g., Premier Biosoft International (see www.premierbiosoft.com/tech_notes/Scorpion.html).

In some embodiments, labels that can be used on the FRET probes include colorimetric and fluorescent dyes, such as Alexa Fluor dyes; BODIPY dyes, such as BODIPY FL, Cascade Blue, and Cascade Yellow; coumarin and its derivatives, such as 7-amino-4-methylcoumarin, aminocoumarin and hydroxycoumarin; cyanine dyes, such as Cy3 and Cy5; cosins and erythrosins; fluorescein and its derivatives, such as fluorescein isothiocyanate; macrocyclic chelates of lanthanide ions, such as Quantum Dye™; Marina Blue; Oregon Green; rhodamine dyes, such as rhodamine red, tetramethylrhodamine and rhodamine 6G; Texas Red; fluorescent energy transfer dyes, such as thiazole orange-ethidium heterodimer; and TOTAB.

Specific examples of dyes include, but are not limited to, those identified above and the following: Alexa Fluor 350, Alexa Fluor 405, Alexa Fluor 430, Alexa Fluor 488, Alexa Fluor 500. Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, and, Alexa Fluor 750; amine-reactive BODIPY dyes, such as BODIPY 493/503, BODIPY 530/550, BODIPY 558/568, BODIPY 564/570, BODIPY 576/589, BODIPY 581/591, BODIPY 630/650, BODIPY 650/655, BODIPY FL, BODIPY R6G, BODIPY TMR, and, BODIPY-TR; Cy3, Cy5, 6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, SYPRO, TAMRA, 2′, 4′,5′,7′-Tetrabromosulfonefluorescein, and TET.

Examples of dye/quencher pairs (i.e., donor/acceptor pairs) include, but are not limited to, fluorescein/tetramethylrhodamine; IAEDANS/fluorescein; EDANS/dabcyl; fluorescein/fluorescein; BODIPY FL/BODIPY FL; and fluorescein/QSY 7 or QSY 9 dyes. When the donor and acceptor are the same, FRET may be detected, in some embodiments, by fluorescence depolarization. Certain specific examples of dye/quencher pairs (i.e., donor/acceptor pairs) include, but are not limited to, Alexa Fluor 350/Alexa Fluor488; Alexa Fluor 488/Alexa Fluor 546; Alexa Fluor 488/Alexa Fluor 555; Alexa Fluor 488/Alexa Fluor 568; Alexa Fluor 488/Alexa Fluor 594; Alexa Fluor 488/Alexa Fluor 647; Alexa Fluor 546/Alexa Fluor 568; Alexa Fluor 546/Alexa Fluor 594; Alexa Fluor 546/Alexa Fluor 647; Alexa Fluor 555/Alexa Fluor 594; Alexa Fluor 555/Alexa Fluor 647; Alexa Fluor 568/Alexa Fluor 647; Alexa Fluor 594/Alexa Fluor 647; Alexa Fluor 350/QSY35; Alexa Fluor 350/dabcyl; Alexa Fluor 488/QSY 35; Alexa Fluor 488/dabcyl; Alexa Fluor 488/QSY 7 or QSY 9; Alexa Fluor 555/QSY 7 or QSY9; Alexa Fluor 568/QSY 7 or QSY 9; Alexa Fluor 568/QSY 21; Alexa Fluor 594/QSY 21; and Alexa Fluor 647/QSY 21. In some instances, the same quencher may be used for multiple dyes, for example, a broad spectrum quencher, such as an Iowa Black® quencher (Integrated DNA Technologies, Coralville, IA) or a Black Hole Quencher™ (BHQ™; Sigma-Aldrich, St. Louis, MO).

Specific examples of fluorescently labeled ribonucleotides useful in the preparation of probes for use in some embodiments of the methods described herein are available from Molecular Probes (Invitrogen), and these include, Alexa Fluor 488-5-UTP, Fluorescein-12-UTP, BODIPY FL-14-UTP, BODIPY TMR-14-UTP, Tetramethylrhodamine-6-UTP, Alexa Fluor 546-14-UTP, Texas Red-5-UTP, and BODIPY TR-14-UTP. Other fluorescent ribonucleotides are available from Amersham Biosciences (GE Healthcare), such as Cy3-UTP and Cy5-UTP.

Specific examples of fluorescently labeled deoxyribonucleotides useful in the preparation of probes for use in the methods described herein include Dinitrophenyl (DNP)-1′-dUTP, Cascade Blue-7-dUTP, Alexa Fluor 488-5-dUTP, Fluorescein-12-dUTP, Oregon Green 488-5-dUTP, BODIPY FL-14-dUTP, Rhodamine Green-5-dUTP, Alexa Fluor 532-5-dUTP, BODIPY TMR-14-dUTP, Tetramethylrhodamine-6-dUTP, Alexa Fluor 546-14-dUTP, Alexa Fluor 568-5-dUTP, Texas Red-12-dUTP, Texas Red-5-dUTP, BODIPY TR-14-dUTP, Alexa Fluor 594-5-dUTP, BODIPY 630/650-14-dUTP, BODIPY 650/665-14-dUTP; Alexa Fluor 488-7-OBEA-dCTP, Alexa Fluor 546-16-OBEA-dCTP, Alexa Fluor 594-7-OBEA-dCTP, and Alexa Fluor 647-12-OBEA-dCTP. Fluorescently labeled nucleotides are commercially available and can be purchased from, e.g., Invitrogen.

As noted above, exemplary detectable moieties also include members of binding pairs. Exemplary binding pairs include, but are not limited to, biotin and streptavidin, antibodies and antigens, etc.

### Sample

The sample to be tested can be any sample suspected of containing at least on the respiratory pathogen biomarkers described herein. In some embodiments, the sample is a biological sample collected from a subject. In other embodiments, the sample is a sample that is not collected directly from a subject, such as, e.g., a wastewater sample or a sample from an air filter in a building.

Illustrative biological samples include samples of bodily fluids, such as nasal aspirates, nasal washes, nasal swabs, nasopharyngeal swabs, saliva, oropharyngeal swabs, throat swabs, bronchoalveolar lavage samples, bronchial aspirates, bronchial washes, endotracheal aspirates, endotracheal washes, tracheal aspirates, nasal secretion samples, mucus samples, sputum samples, lung tissue samples, etc.

The sample to be tested is, in some embodiments, fresh (i.e., never frozen). In other embodiments, the sample is a frozen specimen. In some embodiments, the sample is a tissue sample, such as a formalin-fixed paraffin embedded sample. In some embodiments, the sample is a liquid cytology sample.

In some embodiments, a sample to be tested is contacted with a buffer after collection. For example, in the case of a nasal aspirate sample or nasal wash sample or a sample derived from a nasal aspirate sample or nasal wash sample, a buffer (including, e.g., a preservative) can be added to the nasal aspirate sample or nasal wash sample. In embodiments where the sample is a nasopharyngeal swab sample, the swab can simply be placed in a buffer. In some embodiments, that sample is contacted with the buffer immediately; in the case of a swab, the swab is immediately placed in the buffer. In some embodiments, the sample (e.g., including the swab) is contacted with buffer within 5 minutes, within 10 minutes, within 30 minutes, within 1 hour, or within 2 hours of sample collection.

In some embodiments, less than 5 ml, less than 4 ml, less than 3 ml, less than 2 ml, less than 1 ml, or less than 0.75 ml of sample or buffered sample are used in the present methods. In some embodiments, 0.1 ml to 1 ml of sample or buffered sample is used in the present methods.

### Subjects

A biological sample useful in the methods described herein can be collected from any subject that can be infected by one, several, or all of the respiratory pathogens described above. In various embodiments, the subject can include non-human animals, e.g., canines, felines, equines, primates, and other non-human mammals, as well as humans.

In some embodiments, the sample to be tested is obtained from an individual who has one or more symptoms of influenza infection. Nonlimiting exemplary symptoms of influenza include fever, chills, cough, sore throat, runny nose, nasal congestion, muscle ache, headache, fatigue, vomiting, diarrhea, and combinations of any of these symptoms. In some embodiments, the sample to be tested is obtained from an individual who has previously been diagnosed with a condition caused by a respiratory pathogen (e.g., influenza or Covid-19). In some such embodiments, the individual is monitored for recurrence of a condition caused by a respiratory pathogen (e.g., influenza or Covid-19).

In some embodiments, methods described herein can be used for routine screening of apparently healthy individuals with no risk factors. In some embodiments, methods described herein are used to screen asymptomatic individuals, for example, during routine or preventative care. In some embodiments, methods described herein are used to screen women who are pregnant or who are attempting to become pregnant.

In some embodiments, the methods described herein can be used to assess the effectiveness of a treatment in an individual undergoing treatment a condition caused by a respiratory pathogen (e.g., influenza or Covid-19).

### Assay Methods

Any analytical procedure capable of permitting specific detection of a target nucleic acid can be used in the methods herein presented. In some embodiments, DNA targets can be detected by direct hybridization or, more easily, by amplification of the DNA template and detection of the amplicon. In some embodiments, RNA targets can be detected by direct hybridization or, more easily, by reverse transcribing a target RNA to produce a cDNA that is complementary to the target RNA. This cDNA can be directly detected by direct hybridization or by amplification of the cDNA template.

Nucleic acid amplification provides rapid, sensitive, and specific detection of nucleic acid targets, and has been employed in a wide variety of assay formats to detect nucleic acid targets. Those of skill in the art can, following the guidance herein, carry out the methods described herein in any number of different nucleic acid amplification-based assays, using, for example, any of the nucleic acid amplification methods discussed above. Such methods can entail thermocycling, but need not do so, as in the case of isothermal amplification. Exemplary methods include, but are not limited to, isothermal amplification, real time RT-PCR, endpoint RT-PCR, and amplification using T7 polymerase from a T7 promoter annealed to a DNA, such as provided by the SenseAmp Plus™ Kit available at Implen, Germany. Amplification and detection can be carried out in solution or can make use of a solid support (e.g., a biochip). Nucleic acid amplification-based assays can employ a single reaction chamber or multiple reaction chambers. Amplification can be nested or non-nested. In some embodiments, detection includes electrochemical detection.

In some embodiments, target nucleic acids, such as respiratory and other pathogen biomarkers and/or optional controls, can be detected by (a) contacting nucleic acid from the sample with a set of primers and optional probes for detecting the presence of the desired target nucleic acids, (b) subjecting the nucleic acid, primers, and optional probes to amplification conditions; (c) detecting the presence of any amplification product(s), optionally via real-time PCR, melt curve analysis, or a combination thereof, and (d) differentially identifying the presence of a viral and/or bacterial respiratory pathogen in the sample, or determining that no viral or bacterial pathogen detectable using the set of primers is present, based on detection of the amplification product(s) or lack thereof, respectively. In this context, “differentially identifying” refers to the ability to determine that a particular target organism is present and that one or more other target organisms of the assay are not. In some embodiments, the assay is able to determine the presence of any target organism this present in the sample, while ruling out the presence of the other target organisms (above the detection limit of the assay).

In some embodiments of amplification by polymerase chain reaction (PCR), an exemplary cycle comprises an initial denaturation at 90° C. to 100° C. for 20 seconds to 5 minutes, followed by cycling that comprises denaturation at 90° C. to 100° C. for 1 to 10 seconds, followed by annealing and amplification at 60° C. to 75° C. for 10 to 40 seconds. A further exemplary cycle comprises 20 seconds at 94° C., followed by up to 3 cycles of 1 second at 95° C., 35 seconds at 62° C., 20 cycles of 1 second at 95° C., 20 seconds at 62° C., and 14 cycles of 1 second at 95° C., 35 seconds at 62° C. In some embodiments, for the first cycle following the initial denaturation step, the cycle denaturation step is omitted. In some embodiments, Taq polymerase is used for amplification. In some embodiments, the cycle is carried out at least 10 times, at least 15 times, at least 20 times, at least 25 times, at least 30 times, at least 35 times, at least 40 times, or at least 45 times. In some embodiments, Taq is used with a hot-start function. In some embodiments, detection of the target nucleic acids occurs in less than 3 hours, less than 2.5 hours, less than 2 hours, less than 1 hour, or less than 30 minutes from initial denaturation through the last extension. In some embodiments, target nucleic acids are detected by a method that includes real-time quantitative PCR, e.g., using FRET probes, such as those described above.

In some embodiments, quantitation of the results of real-time PCR assays is done by constructing a standard curve from a nucleic acid of known concentration and then extrapolating quantitative information for target nucleic acids of unknown concentration. In some embodiments, the nucleic acid used for generating a standard curve is a DNA (for example, an endogenous control, or an exogenous control). In some embodiments, the nucleic acid used for generating a standard curve is a purified double-stranded plasmid DNA or a single-stranded DNA generated in vitro.

In some embodiments, in order for an assay to indicate that a given target nucleic acid is not present in a sample, the Ct values for an endogenous control (such as an SAC) and/or an exogenous control (such as an SPC) must be within previously-determined valid ranges. For example, in some embodiments, the absence of a particular target nucleic acid cannot be confirmed unless the controls are detected, indicating that the assay was successful.

In some embodiments, a threshold Ct (or a “cutoff Ct”) value for a target nucleic acid (including an endogenous control and/or exogenous control), below which the gene is considered to be detected, has previously been determined. In some embodiments, a threshold Ct is determined using substantially the same assay conditions and system (such as a GeneXpert®) on which the samples will be tested.

Real-time PCR is performed using any PCR instrumentation available in the art. Typically, instrumentation used in real-time PCR data collection and analysis comprises a thermal cycler, optics for fluorescence excitation and emission collection, and optionally a computer and data acquisition and analysis software.

In some embodiments, the number of target nucleic acids in an assay exceeds the number of labels that can be detected, e.g., in particular instrument. Therefore, the PCR amplification can be followed by a melt analysis to increase the number of possible reported results. In general, target organisms requiring high sensitivity, e.g., viruses, can be detected with real-time PCR detection using TaqMan probes or molecular beacon probes, and target organisms requiring less sensitivity, e.g., bacteria can be detected with melt analysis. However, some viruses, such as metapneumovirus and influenza H1N1, can be detected with melt analysis.

Another approach to detect target nucleic acids can include high-resolution melt alone. For example, the Biofire® FilmArray® System performs a nested multiplex PCR by first performing reverse transcription, followed by a multiplexed first-stage PCR reaction (PCR1). Multiple simultaneous second-stage PCR reactions (PCR2) are then performed in an array to amplify sequences within the PCR1 products. Endpoint melting curve data to detect target nucleic acids and analyses can then be performed to generate a result for each analyte.

Target nucleic acids can also be detected by real-time PCR but in more than one reaction chambers. The QIAstat-Dx Analyzer utilizes 8 real-time PCR reaction chambers with 6-plex capability. Another approach to detect a target nucleic acid can include digital microfluidics or electrowetting and electrochemical detection. For example, the Dx ePlex instrument utilizes digital microfluidics or electrowetting, responsible for the movement and transfer of samples and reagents inside a cartridge. The ePlex system includes a microarray for detection, consisting of target-specific capture probes attached to gold electrodes (solid-support), which generates a voltage signal if a “target DNA/signal probe” hybridizes with the capture probes. Target nucleic acids can also be detected using a chip that includes an integrated sensor array.

Examples of other approaches that can be employed in the methods describe herein include bead-based flow cytometric assay. See Lu J. et al. (2005) Nature 435:834-838, which is incorporated herein by reference for this description. An example of a bead-based flow cytometric assay is the xMAP® technology of Luminex, Inc. See www.luminexcorp.com/technology/index.html. Another approach uses microfluidic devices and single-molecule detection. See U.S. Pat. Nos. 7,402,422 and 7,351,538 to Fuchs et al, U.S. Genomics, Inc., each of which is incorporated herein by reference in its entirety. Yet another approach is simple gel electrophoresis and detection with labeled probes (e.g., probes labeled with a radioactive or chemiluminescent label), such as by northern blotting.

In some embodiments, the approach for detecting a target nucleic acid does not include bead-based flow cytometric assay, microfluidic devices and single-molecule detection, simple gel electrophoresis, use of a capture probe attached to a solid-support, separation of reaction mixture into multiple reaction chambers, array-based detection, nested amplification, electrochemical detection, high resolution melt only, or a combination thereof.

### Automated Assay Methods

Readily automated approaches are of great interest. The methods described herein can be carried out in a substantially automated manner using a commercially available nucleic acid amplification system. Exemplary nonlimiting nucleic acid amplification systems that can be used to carry out the methods of the invention include the GENEXPERT® system, a GENEXPERT® Infinity system, and GENEXPERT® Xpress System (Cepheid, Sunnyvale, Calif.). In some embodiments, the amplification system may be available at the same location as the individual to be tested, such as a health care provider's office, a clinic, or a community hospital, so processing is not delayed by transporting the sample to another facility. Assays according to the method described herein can be completed in under 3 hours, in some embodiments, under 2 hours, in some embodiments, under 1 hour, in some embodiments, under 45 minutes, in some embodiments, under 35 minutes, and in some embodiments, under 30 minutes, using an automated system, for example, the GENEXPERT® system. The GENEXPERT® utilizes a self-contained, single-use cartridge. Sample extraction, amplification, and detection may all carried out within this self-contained sample cartridge as described herein.

In some embodiments, after the sample is added to the cartridge, the sample is contacted with lysis buffer and released nucleic acid (NA) is bound to a NA-binding substrate, such as a silica or glass substrate. The sample supernatant is then removed and the NA eluted in an elution buffer, such as a Tris/EDTA buffer. The eluate may then be processed in the cartridge to detect target nucleic acids as described herein. In some embodiments, the eluate is used to reconstitute at least some of the PCR reagents, which are present in the cartridge as lyophilized particles.

A cartridge having a plurality of chambers can have the set of primers and optional probes described herein, or a subset thereof, disposed in a chamber. In some embodiments, the set of primers and optional probes described herein, or a subset thereof, are disposed in more than one of the plurality of chambers.

In some embodiments, RT-PCR is used to amplify and analyze the presence of the target nucleic acids. In some embodiments, the reverse transcription uses MMLV and/or CAT-A RT enzyme and an incubation of 5 to 20 minutes at 40° C. to 50° C. In some embodiments, the PCR uses Taq polymerase with hot-start function, such as AptaTaq (Roche). In some embodiments, the initial denaturation is at 90° C. to 100° C. for 20 seconds to 5 minutes; the cycling denaturation temperature is 90° C. to 100° C. for 1 to 10 seconds; the cycling anneal and amplification temperature is 60° C. to 75° C. for 10 to 40 seconds; and up to 50 cycles are performed.

In some embodiments, a double-denature method is used to amplify low-copy number target nucleic acids. A double-denature method comprises, in some embodiments, a first denaturation step followed by addition of primers and/or probes for detecting target nucleic acids. All or a substantial portion of the nucleic acid-containing sample (such as a DNA eluate) is then denatured a second time before, in some instances, a portion of the sample is aliquotted for cycling and detection of the target nucleic acids. While not intending to be bound by any particular theory, the double-denature protocol may increase the chances that a low-copy number target nucleic acid (or its complement) will be present in the aliquot selected for cycling and detection because the second denaturation effectively doubles the number of target nucleic acids (i.e., it separates the target nucleic acid and its complement into two separate templates) before an aliquot is selected for cycling. In some embodiments, the first denaturation step comprises heating to a temperature of 90° C. to 100° C. for a total time of 30 seconds to 5 minutes. In some embodiments, the second denaturation step comprises heating to a temperature of 90° C. to 100° C. for a total time of 5 seconds to 3 minutes. In some embodiments, the first denaturation step and/or the second denaturation step is carried out by heating aliquots of the sample separately. In some embodiments, each aliquot may be heated for the times listed above. As a non-limiting example, a first denaturation step for an NA-containing sample (such as a DNA eluate) may comprise heating at least one, at least two, at least three, or at least four aliquots of the sample separately (either sequentially or simultaneously) to a temperature of 90° C. to 100° C. for 60 seconds each. As a non-limiting example, a second denaturation step for a NA-containing sample (such as a DNA eluate) containing enzyme, primers, and probes may comprise heating at least one, at least two, at least three, or at least four aliquots of the eluate separately (either sequentially or simultaneously) to a temperature of 90° C. to 100° C. for 5 seconds each. In some embodiments, an aliquot is the entire NA-containing sample (such as a DNA eluate). In some embodiments, an aliquot is less than the entire NA-containing sample (such as a DNA eluate).

In some embodiments, an off-line centrifugation is used, for example, with samples with low cellular content. The sample, with or without a buffer added, is centrifuged and the supernatant removed. The pellet is then resuspended in a smaller volume of either supernatant or the buffer. The resuspended pellet is then analyzed as described herein.

### Exemplary Automation and Systems

Many existing fully integrated nucleic acid amplification and test systems capable of sample preparation are normally quite complicated and costly. The nucleic acid amplification and test systems provided herein perform rapid, simple, convenient, and affordable nucleic acid analysis.

### System Overview

In one aspect, the invention pertains to a sample cartridge that utilizes a valve body platform that allows for detection of enveloped and free target nucleic acids. In some embodiments, the valve body includes a sample processing region or lysing chamber that provides for either or both mechanical and chemical lysis. This allows a single cartridge to provide lysing for a multitude of differing types of targets, thus, can be considered an “panel assay cartridge.” In some embodiments, the sample cartridge can perform processing and detection of both bacterial targets requiring mechanical lysing and viral targets suited for chemical lysing.

The sample cartridge device can be any device configured to perform one or more process steps relating to preparation and/or analysis of a biological fluid sample according to any of the methods described herein. In some embodiments, the sample cartridge device is configured to perform at least sample preparation. The sample cartridge can further be configured to perform additional processes, such as detection of a target nucleic acid in a nucleic acid amplification test (NAAT), e.g., Polymerase Chain Reaction (PCR) assay, by use of a reaction vessel attached to the sample cartridge. In some embodiments, the reaction vessel extends from the body of the cartridge. Preparation of a fluid sample generally involves a series of processing steps, which can include chemical, electrical, mechanical, thermal, optical or acoustical processing steps according to a specific protocol. Such steps can be used to perform various sample preparation functions, such as cell capture, cell lysis, binding of analyte, and binding of unwanted material.

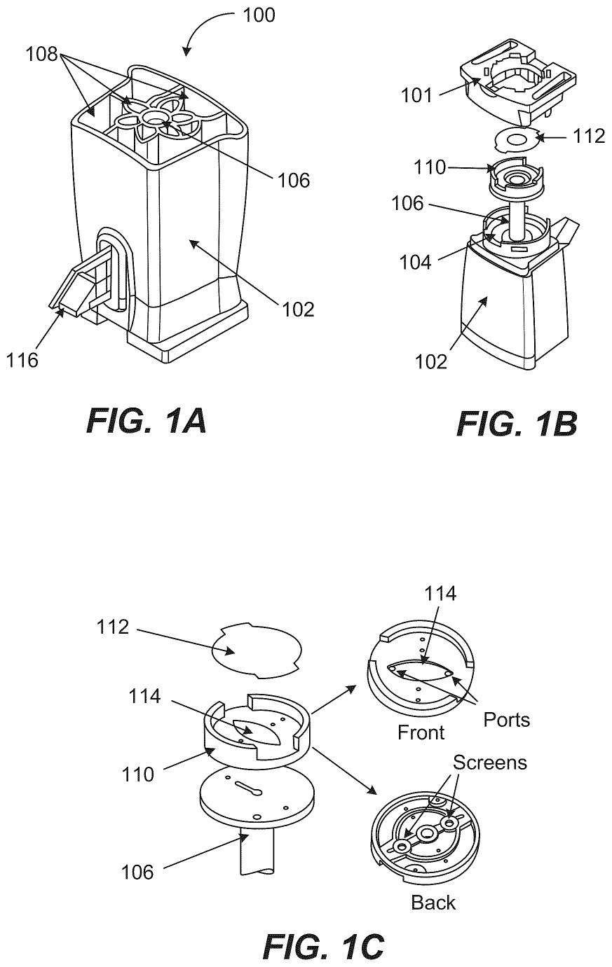

A sample cartridge suitable for use with the invention, includes one or more transfer ports through which the prepared fluid sample can be transported into an attached reaction vessel for analysis. FIG. 1A illustrates an exemplary panel assay cartridge 100 suitable for sample preparation and analytics testing by PCR when received in an instrument module in accordance with some embodiments. The sample cartridge is attached with a reaction vessel 116 (also referred to as a “reaction tube” or “PCR tube”) adapted for analysis of a fluid sample processed within the sample cartridge 100. In some embodiments the reaction vessel extends from the cartridge body. Such a sample cartridge 100 includes various components including a main housing 102 having one or more chambers 108 for processing of the fluid sample, which typically include sample preparation before analysis. In these embodiments, the sample cartridge can be a fully integrated nucleic acid amplification and test system combining sample preparation, amplification, and detection together. The instrument module facilitates the processing steps needed to perform sample preparation and the prepared sample is transported through one of a pair of transfer ports into fluid conduit of the reaction vessel 116 attached to the housing of the sample cartridge 100. The prepared biological fluid sample is then transported into a reaction chamber of the reaction vessel where the biological fluid sample undergoes nucleic acid amplification. In some embodiments, the amplification is a polymerase chain reaction. In some embodiments, concurrent with the amplification of the biological fluid sample, an excitation means, and an optical detection means of the module is used to detect optical emissions that indicate the presence or absence of a target nucleic acid analyte of interest, e.g., a bacterium, a virus, a pathogen, a toxin, or other target analyte. It is appreciated that such a reaction vessel could include various differing chambers, conduits, or micro-well arrays for use in detecting the target analyte. The sample cartridge can be provided with means to perform preparation of the biological fluid sample before transport into the reaction vessel. Any chemical reagent required for viral or cell lysis or means for binding or detecting an analyte of interest (e.g., reagent beads) can be contained within one or more chambers of the sample cartridge, and as such can be used for sample preparation.

An exemplary use of a reaction vessel for analyzing a biological fluid sample is described in commonly assigned U.S. Pat. No. 6,818,185, entitled “Cartridge for Conducting a Chemical Reaction,” filed May 30, 2000, the entire contents of which are incorporated herein by reference for all purposes. Examples of the sample cartridge and associated modules are shown and described in U.S. Pat. No. 6,374,684, entitled “Fluid Control and Processing System” filed Aug. 25, 2000, and U.S. Pat. No. 8,048,386, entitled “Fluid Processing and Control,” filed Feb. 25, 2002, U.S. Patent Application No. 63/217,672 entitled “Universal Assay Cartridge and Methods of Use” filed Jul. 1, 2021; U.S. Provisional Application No. 63/319,993 entitled “Unitary Cartridge Body and Associated Components and Methods of Manufacture” filed Mar. 15, 2022; and U.S. Pat. No. 10,562,030 entitled “Molecular Diagnostic Assay System” filed Jul. 22, 2016; the entire contents of which are incorporated herein by reference in their entirety for all purposes. The above noted patents are included in the attached appendix.

Various aspects of the sample cartridge 100 can be further understood by referring to U.S. Pat. No. 6,374,684 “the '684 patent”), which described certain aspects of a sample cartridge in greater detail. Such sample cartridges can include a fluid control mechanism, such as a rotary fluid control valve assembly, that is fluidically connected to the chambers of the sample cartridge. The term “chamber” can be used interchangeably with the terms “well”, “tube”, and the like. Rotation of the rotary fluid control valve permits fluidic communication between chambers and the valve so as to control flow of a biological fluid sample deposited in the cartridge into different chambers in which various reagents can be provided according to a particular protocol as needed to prepare the biological fluid sample for analysis. To operate the rotary valve, the cartridge processing module comprises a motor such as a stepper motor that is typically coupled to a drive train that engages with a feature of the valve in the sample cartridge to control movement of the valve in coordination with movement of the syringe, thereby resulting movement of the fluid sample according to the desired sample preparation protocol. The fluid metering and distribution function of the rotary valve according to a particular sample preparation protocol is demonstrated in the '684 patent.

### Exemplary Assay Cartridge and Valve Assemblies

### Overview

As shown in FIG. 1A, the panel assay cartridge 100 comprises a cartridge body 102 containing a plurality of chambers 108 for reagents or buffers and sample processing. The chambers are disposed around a central syringe barrel 106 that is in fluid communication with a valve body 110 (see FIGS. 1B and 1C) and that is sealed with a gasket 104. The valve body 110 can include a cap 112 and the entire cartridge body can be supported on a cartridge base 101. The valve body typically contains one or channels or cavities (chamber(s) 114) that can contain a filter as described herein that can function to bind and elute a nucleic acid. In some embodiments the cartridge further comprises one or more temperature-controlled channels or chambers that can, in certain embodiments, function as thermocycling chambers. A “plunger” not shown can be operated to draw fluid into the syringe barrel 106 and rotation of the valve body 110 provides selective fluid communication between the various reagent chambers 108 and channels, reaction chamber(s), mixing chambers, and optionally, any temperature controlled regions. Thus, the various reagent chambers 108, reaction chambers, filter material(s), and temperature-controlled chambers or channels are selectively in fluid communication by rotation of the plunger and reagent movement (e.g., chamber loading or unloading) is operated by the “syringe” action of the plunger within the valve assembly. In other embodiments, the various reagent chambers, reaction chambers, filter material, and temperature-controlled chambers or channels are selectively in fluid communication by linear progression (e.g., by forced movement) of the reagents and sample from one chamber to the next.

### Exemplary Valve Assemblies

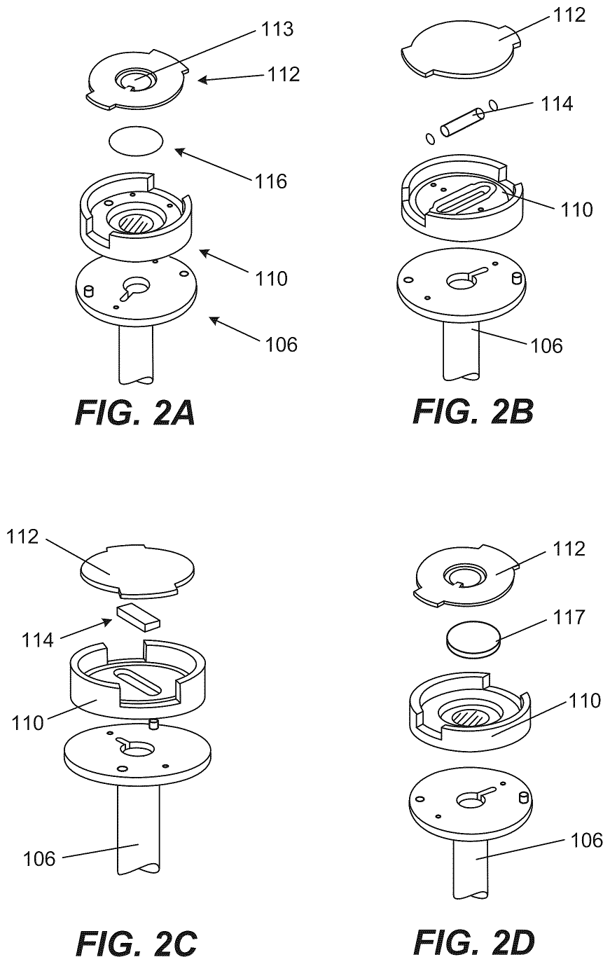

FIG. 2 illustrates differing valve assembles that can be used in the sample cartridge of FIG. 1A. Each of the valve assemblies is shown inverted to better illustrate the components of the valve body. Valve assembly A performs only mechanical lysing and is suitable for lysing hardy targets (e.g. certain bacteria, spores). Valve assemblies B and C perform only chemical lysing and is suitable for lysing less hardy targets (e.g. viruses, free NA, some spores, some bacteria and yeasts). Valve assembly D can perform both mechanical and chemical lysing for all types of targets. In all such cartridges, the valve assembly includes the syringe tube 106, valve body 110, and valve cap 112. In one aspect, the additional capabilities of the valve assembly of the sample cartridge rely in part on the filter, features of the valve body and cap, as well as the particular workflow sequence performed by the instrument interface of the module. For example, valve assembly A has a valve body 110 shaped with a circular cavity to support a polymer filter disc 116 to filter the sample, and the cap 112 has a sonication dome feature 113, which interfaces with a sonication horn of a cartridge receiving module so as to ultrasonically lyse the target. By contrast, valve assemblies B and C have a valve body 110 with an oblong filter recess 111 that receives a glass filter 114 (e.g. glass column) therein, the glass filter configured for binding with nucleic acid released from the target by chemical lysing. Valve assembly D has a design more similar to valve assembly A, having a cap 112 with a sonication dome 113, and a valve body with a circular cavity for supporting a disc filter, however this design uses a glass fiber filter 117. Utilizing glass fibers to form the filter facilitates affinity bonding with the free nucleic acid released by chemical lysing. Thus, this filter is suited for both mechanical and chemical lysing. U.S. Provisional Application No. 63/217,672 further details the assay cartridge of valve assembly D and is incorporated herein by reference in its entirety.

### Reaction Modules

In certain embodiments the cartridge 200 is configured for insertion into a reaction module 300. The module is configured to receive the cartridge 200 therein. In certain embodiments the reaction module provides heating plates 308 to heat the temperature controlled chamber or channel. The module can optionally additionally include a fan 304 to provide cooling where the temperature controlled channel or chamber is a thermocycling channel or chamber. Electronic circuitry 302 can be provided to pass information (e.g., optical information) top a computer for analysis. In certain embodiments the module can contain optical blocks 306 to provide excitation and/or detection of one or more (e.g., 1, 2, 3, 4, or more) optical signals representing, e.g., signal DNAs amplified for various PCR targets. In various embodiments an electrical connector 312 can be provided for interfacing the module with a system (e.g. system controller or with a discrete analysis/controller unit. The sample can be introduced into the cartridge using a pipette 310. In certain embodiments, the module also contains a controller that operates a plunger in the syringe barrel and the rotation of the valve body.

### Analytical System

In certain embodiments a system (e.g., a processing unit) is provided. The system includes an enclosure that is configured to support and power multiple sample processing modules 300, where each processing module is configured to hold and operate a removable cartridge 100. In some embodiments, the system is configured to operate the sample processing modules to perform a PCR assay for one or more target nucleic acid analytes and optionally to determine the level of one or more target RNA/DNA sequences within a corresponding removable sample cartridge. Typically, the processing on a sample within the corresponding removable sample cartridge involves operating the cartridge to perform a method as described herein. In certain embodiments the system is configured to contain one sample processing module. In certain embodiments the system is configured to contain at least two or more sample processing modules (e.g., at least 4, 8, 12, 16, 20, 24, 28, 32, 64, 128 or more) sample processing modules. In some embodiments, the system provides a user interface that allows the user input operational instructions and/or to monitor operation of the cartridges to determine the presence and/or quantity of one or more nucleic acids.

While the methods described herein are described primarily with reference to the GENEXPERT® cartridge by Cepheid Inc. (Sunnyvale, Calif.) (see, e.g., FIG. 1A), it will be recognized, that in view of the teachings provided herein the methods can be implemented on other cartridge/microfluidic systems, including alternative cartridge designs having valve assemblies that involve multiple interfacing components, as well as cartridge body defined by multiple interfacing components to form the multiple chambers of the cartridges, for example, those described in Korean Application No. 102293717B1 and KR102362853B1, cartridges that utilizes ultrasonic waves to lyse cells in a biological sample, for example, those described in International Application No. WO2021/245390A1, cartridges and systems that utilizes an electrowetting grid for microdroplet manipulation and electrosensor arrays configured to detect analytes of interest, for example, those described in International Application No. WO2016/077341A2, cartridges that facilitate movement of nucleic acid from one chamber to the next chamber by opening a vent pocket, for example, those described in International Application No. WO2012/145730A2, multiplexed assay systems comprising a plurality of thermocycling units such that individual chambers can be heated, cooled, and/or compressed to mix fluid within the chamber or to propel fluid in the chamber into another chamber, for example, those described in International Application No. WO2015/138343A1, and as well as systems for rapid amplification of nucleic acids facilitated by flexible portions of the sample cartridge aligned to accomplish temperature cycling for nucleic acid amplification, for example, those described in International Application No. WO2017/147085A1. Such cartridge/microfluidic systems can include, for example microfluidic systems implemented using soft lithography, micro/nano-fabricated microfluidic systems implemented using hard lithography, and the like.

In one exemplary embodiment, the cartridge can include a plurality of cartridge bodies, such as a first body, a second body, a central syringe barrel that is in fluid communication with the first body and the second body, a reaction vessel, and the like. The first body may be formed of a plurality of chambers separated from each other for reagents or buffers and sample processing. In some embodiments, the first body can be used for the purpose of storing a plurality of reagents. The second body may be formed of one or a plurality of chambers separated from each other and includes a path through which the reagent or sample from the first body moves. When the first body and the second body of cartridge are combined, a liquid flow path and optionally an air flow path can be formed between both compartments via the central syringe barrel. The liquid flow path is connected to the first body to provide a space for samples and reagents to move and mix. The air flow path may connect the reaction vessel and a vacuum control region of the “plunger” to control the vacuum that may occur when the extracted nucleic acid moves to the reaction vessel. Rotation of the syringe barrel comprising a “plunger” that can sequentially suck sample and reagents from the plurality of chambers into an interior space of the syringe barrel and discharge the mixture of the interior space into any one of the plurality of chambers (first body or second body) of the cartridge. Rotation of the syringe barrel comprising a “plunger” can suck the reagent inside the plurality of chambers of the cartridge into the interior space of the syringe barrel and then discharge the mixed reagent to a nucleic acid amplification reaction vessel.

In another exemplary embodiment, the cartridge includes a flow cover and a base plate, which together form a closed passage therein. In one embodiment of this configuration, an inner chamber containing the reagents required for dielectric extraction is provided separately from an outer chamber, and the upper and lower portions of the inner chamber are sealed. In addition, a double-structured flow cover-pad can be disposed between the outer chamber and the base plate. Closed flow paths are formed by achieving a strong coupling between the base plate-the flow cover-the pad-the outer chamber. Also provided in this configuration are beads necessary for dielectric extraction and amplification which are accommodated in a dual chamber structure of an outer chamber-bead chamber. The beads can be maintained by a dehumidifying unit positioned above the bead chamber even when the bead chamber is opened.

In further exemplary embodiment, the cartridge can include a plurality of reaction chambers, particularly, the reaction vessel can include a plurality of reaction chambers. In these embodiments, different types of lyophilized primers and probes can be provided in each reaction chamber. For example, primers and probes for viral-associated nucleic acids can be provided in one reaction chamber, and primers and probes for bacterial-associated nucleic acids can be provided in a second chamber for amplification and detection, and such the like. Of course, it is possible to perform various amplification and detection processes at the same time in a single reaction chamber. Accordingly, amplification of each target nucleic acid described herein may be performed individually in separate reaction chambers or wells or carried out in a multiplex reaction in a single reaction chamber or well.

Additionally, it is appreciated that the panel assay methods described herein (i.e., identification of multiple conditions based on comparative levels of multiple-target nucleic acids obtained from a single sample) can further be realized in entirely different systems, including: isothermal nucleic acid amplification systems, digital RT-PCR, electrochemical PCR, lateral flow testing cartridges, electrochemical sensors, nucleic acid sequencing, CRISPR/Cas based technologies, chemiluminescence, and nanoparticle-based colorimetric detection.

In various embodiments, the signal DNA(s) from PCR (nucleic acid amplification) reactions are amplified for detection and/or quantification. In certain embodiments, the amplification comprise any of a number of methods including, but not limited to polymerase chain reaction (PCR), ligase chain reaction (LCR), ligase detection reaction (LDR), multiplex ligation-dependent probe amplification (MLPA), ligation followed by Q-replicase amplification, primer extension, strand displacement amplification (SDA), hyperbranched strand displacement amplification, multiple displacement amplification (MDA), nucleic acid strand-based amplification (NASBA), rolling circle amplification (RCA), and the like.

In illustrative, but non-limiting embodiments, the amplification reaction may produce an optical signal that is proportional to the amount of amplified target nucleic acid (e.g., signal DNA). Illustrative optical signals include, but are not limited to a fluorescent signal, a chemiluminescent signal, an electrochemiluminescent signal, a colorimetric signal, and the like. In certain embodiments the optical signal is a fluorescent optical signal generated by a fluorescent indicator. In certain embodiments the fluorescent indicator is a non-specific intercalating dye that binds to double-stranded DNA products, while in certain other embodiments, the fluorescent indicator comprises a target sequence-specific probe (e.g., a TAQMAN® probe, a SCORPION® probe, a MOLECULAR BEACON®, and the like).

Single PCR reactions (nucleic acid amplification), or multiple PCR reactions (nucleic acid amplifications) run sequentially (or simultaneously in separate temperature controlled channels or chambers) can also use the same detectable label since sequentially run PCR signal DNAs are analyzed sequentially and the simultaneous PCR signal DNAs are distinguished by the occurrence in different temperature controlled channels or chambers. The signal produced by this amplification can be distinguished from other amplification products because it is not run at the same time and/or because it is run in a different reaction channel/chamber. However, where multiple nucleic acid amplifications are run simultaneously in the same chamber the reaction products of for each analysis are typically detected and/or quantified by the use of different and distinguishable labels.

In certain embodiments, amplification products (amplified nucleic acid from nucleic acid analysis) can be detected using methods well known to those of skill in the art. In certain embodiments the amplification is a straightforward simple PCR amplification reaction. In certain embodiments, however, a nested PCR reaction is used to amplify the nucleic acid from the nucleic acid analysis. In various embodiments, multiplexed PCR assays are contemplated, particularly where it is desired to analyze multiple products of the nucleic acid analysis in the same amplification reaction. In certain embodiments in such multiplexed amplification reactions, each probe (e.g., for each specific analyte) has its own specific dye/fluor so that it is detectable independently of the other probes. In certain embodiments, typically, for signal generation, the probes used in various amplification reactions utilize a change in the fluorescence of a fluorophore due to a change in its interaction with another molecule or moiety brought about by changing the distance between the fluorophore and the interacting molecule or moiety for detection and/or quantification of the amplified product. Alternatively, other methods of detecting a polynucleotide in a sample, including, but not limited to, the use of radioactively labeled probes, are contemplated.

### Assay Workflows

Prior to carrying out amplification reactions on a sample, one or more sample preparation operations are performed on the sample. Typically, these sample preparation operations will include such manipulations as extraction of intracellular material, e.g., nucleic acids from whole cell samples, viruses and the like to form a crude extract, additional treatments to prepare the sample for subsequent operations, e.g., denaturation of contaminating (e.g., DNA binding) proteins, purification, filtration, desalting, and the like. Liberation of nucleic acids from the sample cells or viruses, and denaturation of DNA binding proteins may generally be performed by chemical, physical, or electrolytic lysis methods. For example, chemical methods generally employ lysing agents to disrupt the cells and extract the nucleic acids from the cells, followed by treatment of the extract with chaotropic salts such as guanidinium isothiocyanate or urea to denature any contaminating and potentially interfering proteins. Generally, where chemical extraction and/or denaturation methods are used, the appropriate reagents may be incorporated within a sample preparation chamber, a separate accessible chamber, or may be externally introduced. Preferably, sample preparation is carried out in only one step or no more than two steps. For example, sample preparation can include heating the sample in a lysis solution without further purification prior to carrying out the amplification reaction. In some embodiments, the lysed sample may be diluted prior to carrying out the amplification reaction. One or more of these various sample preparation operations are readily incorporated into the fluidly closed cartridge systems contemplated herein.

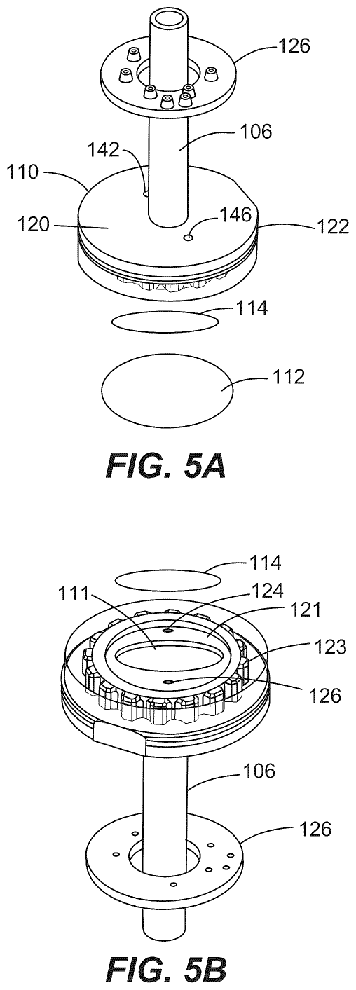

FIGS. 5A-C show illustrative but non-limiting workflows for nucleic acid amplification. In one aspect, the assay sample cartridge, as described herein, is capable of a specialized workflow that performs lysing and detection of differing target analytes as required for a particular panel assay. In some embodiments, the cartridge is configured for chemical lysing of the multiple target organisms. In other embodiments, the cartridge is configured for mechanical lysing of the multiple target organisms. In still other embodiments, the cartridge is configured for both mechanical and chemical lysing to allow lysing of multiple targets of differing types. Accordingly, the sample cartridge can be configured to perform the panel assay by an existing workflow associated with conventional cartridges, or can be operated according to an new workflow specially configured for the panel assay.

In one aspect, the sample cartridge having a valve assembly as described in FIG. 2D herein, is capable of a variety of workflows that perform: chemical lysing of target organisms, mechanical lysing of target organisms, or both. Accordingly, the sample cartridge can perform an existing workflow associated with conventional specialized cartridges or can perform entirely new workflows that perform both.

Exemplary assay workflows that can be performed with a single universal cartridge, in accordance with some embodiments, are shown in FIG. 5. In any of these embodiments, the filter can be formed of glass filter to promote affinity binding of the nucleic acids (NA) to the glass fibers and a pore size suited for chemical lysing as well. In any of these workflows, the nucleic acid amplification can be PCR, real-time PCR, isothermal amplification (including but not limited to nucleic acid sequence-based amplification, loop-mediated isothermal amplification, helicase-dependent amplification, rolling circle amplification, multiple displacement amplification, whole genome amplification or recombinase polymerase amplification) or other nucleic acid amplification methods known to persons of skill in the art.

In Workflow A, the sample is optionally exposed to a sample treatment or chemically lysed, then the treated or lysed fluid sample is flowed through the filter where target organisms are captured. In some embodiments, the sample treatment is used to either weaken the cell wall or to inactivate the sample or make it less viscous to facilitate being processed through the filter. The filter is then washed, leaving the target organisms on the filter. Next, the target organisms are mechanically lysed, such as by sonication, to release nucleic acid (NA). In some embodiments, mechanical lysing includes in-filling glass beads along the filter to aid in mechanical lysing of the target. Next, the NA is eluted from the filter and then nucleic acid amplification is performed is performed.

In Workflow B, the sample is chemically lysed to obtain the NA targets. In some embodiments, after chemically lysing, the NA is bound to the filter by the presence of precipitating and binding reagent. Next, the filter is washed with a rinse reagent while the NA remains bound to the filter. Typically, the wash reagents have some amount of salt which still promotes the binding of the NA to the filter, while allowing removal of non-target materials. Next, the filter is eluted to remove the NA targets. In some embodiments, the elution is performed with a PH neutral buffer or basic buffer fluid. The target NA is then delivered to an attached reaction vessel to perform nucleic acid amplification.

In Workflow C, the fluid sample is exposed to sample treatment and/or chemically lyse the target organisms. Next, the NA freed by chemical lysing is bound to the filter. This step may utilize precipitating and binding reagent. Next, the filter is washed with a rinse reagent while the NA remains bound to the filter. Typically, the wash reagents have some amount of salt which still promotes the binding of the NA to the filter, while allowing removal of non-target materials. Next, the target organisms captured in the filter are heat and/or mechanically lysed. This may utilize sonication, and may further utilize glass beads to facilitate mechanical lysing of select target organisms. Then, the lysed target NA is eluted from the filter. In some embodiments, the elution is performed with a pH neutral buffer or basic buffer fluid. The target NA is then delivered to an attached reaction vessel to perform nucleic acid amplification. Thus, in this workflow, the workflow allows for lysing of multiple differing target organisms, some requiring only chemically lysing (e.g. viral targets), and others requiring mechanical lysing (e.g. bacteria, spores, etc.), such that all these target NAs can be released from a single sample and tested by the same sample cartridge. While the above workflow described mechanical lysing after chemical lysing, it is appreciated that other workflows may be utilized in which chemical lysing occurs after mechanical lysing.

In some embodiments, the sample cartridge includes an identifier with information as to the appropriate workflow needed for a particular panel of assays, so that an instrument module receiving the sample cartridge operates according to the specified workflow.

### Exemplary Assay Configurations

### Reagents for Respiratory Biomarker Panel Assay

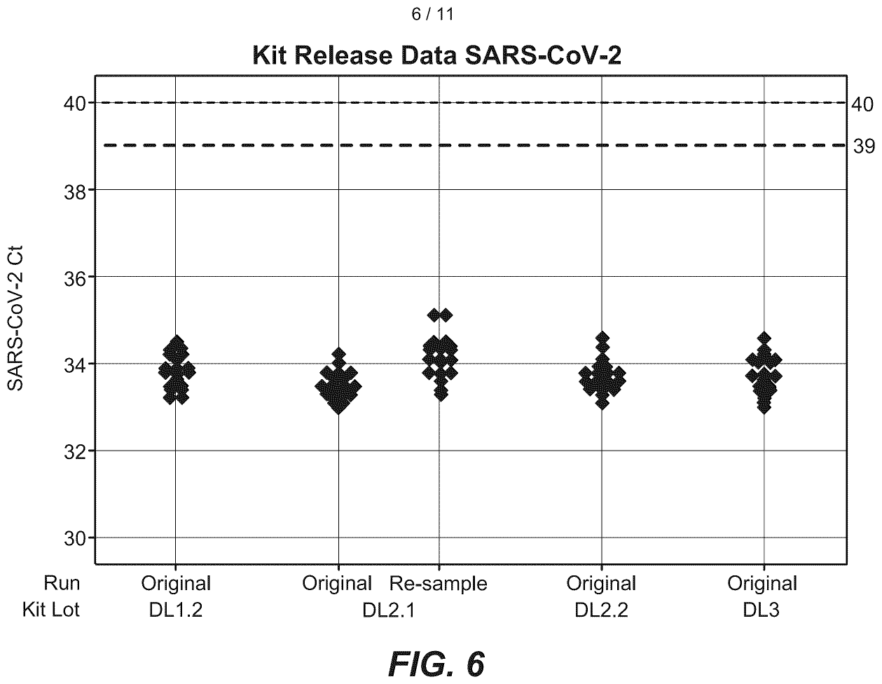

FIG. 6 shows an exemplary sample cartridge than can be used as part of the GENEXPERT® system, with the various chambers designated by number. The exemplary cartridge can include: lyophilized reagents in the form of one or more beads as described herein; an optional lysis reagent; alkaline agent, optional binding reagent, filtering reagent, washing reagent, and eluting reagent. The last four reagents are named according to the functions they perform with respect to nucleic acid. Thus, for example the binding reagent facilitates the binding of nucleic acid to a substrate, the filtering reagent facilitates filtration of the nucleic acid.

In some embodiments, the lysis reagent can include a chaotropic agent, a chelating agent, a buffer, an alkaline agent, or a detergent. The chaotropic agent can be selected from a guanidinium compound such as guanidinium thiocyanate or guanidinium hydrochloride, an alkali perchlorate such as lithium perchlorate, an alkali iodide, magnesium chloride, urea, thiourea, a formamide, or a combination thereof. The concentration of the chaotropic agent can range from about 1 M to about 10 M, such as from about 2.5 M to about 7.5 M, or less than 4.5 M, less than 2 M, or less than 1 M. The chelating agent can be selected from N-acetyl-L-cysteine, ethylenediaminetetraacetic acid (EDTA), diethylene triamine pentaacetic acid (DTPA), ethylenediamine-N,N′-disuccinic acid (EDDS), 1,2-bis(o-aminophenoxy)ethane-N,N,N′,N′-tetraacetic acid (BAPTA), and a phosphonate chelating agent. The concentration of the chelating agent can range from about 10 mM to about 100 mM and/or comprises about 0.5% to about 5% of the lysis reagent. The buffer can be selected from the group consisting of Tris, phosphate buffer, PBS, citrate buffer, TAPS, Bicine, Tricine, TAPSO, HEPES, TES, MOPS, PIPES, Cacodylate, SSC, and MES. The concentration of the buffer can range from about 5 mM to about 100 mM, such as from about 5 mM to about 50 mM. The detergent can be selected from an ionic detergent or a non-ionic detergent. In some examples, the detergent comprises a detergent selected from the group consisting of N-lauroylsarcosine, sodium dodecyl sulfate (SDS), cetyl methyl ammonium bromide (CTAB), TRITON®-X-100, n-octyl-β-D-glucopyranoside, CHAPS, n-octanoylsucrose, n-octyl-β-D-maltopyranoside, n-octyl-β-D-thioglucopyranoside, PLURONIC® F-127, TWEEN® 20, and n-heptyl-β-D-glucopyranoside. The detergent can comprise about 0.1% to about 2% of the lysis reagent, and/or ranges from about 10 mM up to about 100 mM. The lysis reagent can have a pH ranging from about pH 3.0 to about pH 5.5.

In some embodiments, the assays disclosed herein do not utilize a chaotropic agent or a lysis buffer. When a chaotropic agent or lysis buffer is not used, the sample can be contacted with a buffer (or filtering reagent) including, for example, saline (including one or more inorganic salts, such as CaCl₂), MgSO₄, KCl, NaHCO₃, NaCl, etc.), phosphate buffer, Tris buffer, 2-amino-2-hydroxymethyl-1,3-propanediol, HEPES, PBS, citrate buffer, TES, MOPS, PIPES, Cacodylate, SSC, MES, saccharide or disaccharide, or combinations thereof. For example, the buffer can be a commercially available buffer such as Hanks' Balanced Salt Solution available from Sigma Aldrich or TE Buffer available from Fisher BioReagents.

In some embodiments, the alkaline agent can be selected from an alkali metal hydroxide, such as sodium hydroxide or potassium hydroxide. The concentration of the alkaline agent can be about 0.5 N to 5 N.

The binding reagent can promote binding of nucleic acids to the filter, facilitating the removal of non-target material. In some embodiments, the binding reagent can include a binding polymer such as polyacrylic acid (PAA), polyacrylamide (PAM), polyethylene glycol (PEG), poly(sulfobetaine), or a salt, or combinations thereof. In some embodiments, the filtering reagent and/or the washing reagent can include the binding reagent. For example, the binding reagent, the filtering reagent, and/or the washing reagent can include a binding polymer (e.g., PEG 200), buffer, inorganic salt, antioxidant and/or chelating agent, antifoam SE15, sodium azide, disaccharide or disaccharide derivative, carrier protein, detergent, or DMSO. The binding polymer can be present in an amount of at least 10% v/v, at least 20% v/v, at least 30% v/v, and/or less than 60% v/v, less than 40% v/v, less than 30% v/v, less than 20% v/v, or less than 10% v/v or can fall within any range bounded by any of these values, e.g., from 10% to 60% v/v, of the binding reagent, filtering reagent, and/or the washing reagent. The buffer can be selected from the group consisting of Tris, 2-amino-2-hydroxymethyl-1,3-propanediol, HEPES, phosphate buffer, PBS, citrate buffer, TAPS, Bicine, Tricine, TAPSO, HEPES, TES, MOPS, PIPES, Cacodylate, SSC, and MES. The concentration of the buffer can range from about 5 mM to about 100 mM, such as from about 5 mM to about 50 mM. The salt, such as NaCl, KCl, or MgCl₂, can be present at a concentration from about 0.05 M to about 1 M, such as from about 0.1 M to about 0.5 M. The antioxidant and/or chelating agent comprises an agent selected from the group consisting of N-acetyl-L-cysteine, ethylenediaminetetraacetic acid (EDTA), diethylene triamine pentaacetic acid (DTPA), ethylenediamine-N,N′-disuccinic acid (EDDS), 1,2-bis(o-aminophenoxy)ethane-N,N,N′,N′-tetraacetic acid (BAPTA), and a phosphonate chelating agent. In some embodiments the antioxidant and/or chelating agent comprises EDTA. In certain embodiments the antioxidant and/or chelating agent comprise 0.2% to about 5%, about 0.2% to about 3%, or about 0.5% to about 2%, or about 0.5% of the binding reagent, filtering reagent, and/or the washing reagent. In some embodiments the concentration of the antioxidant and/or chelating agent in the binding reagent, filtering reagent, or the washing reagent ranges from about 2 mM to about 50 mM or about 5 mM to about 20 mM. In some embodiments, the detergent is an ionic detergent or a non-ionic detergent. The detergent can be selected from an ionic detergent or a non-ionic detergent. In some examples, the detergent comprises a detergent selected from the group consisting of N-lauroylsarcosine, sodium dodecyl sulfate (SDS), cetyl methyl ammonium bromide (CTAB), TRITON®-X-100, n-octyl-β-D-glucopyranoside, CHAPS, n-octanoylsucrose, n-octyl-β-D-maltopyranoside, n-octyl-β-D-thioglucopyranoside, PLURONIC® F-127, TWEEN® 20, Brij-35, and n-heptyl-β-D-glucopyranoside. The detergent can comprise about 0.1% to about 2% of the binding reagent, filtering reagent, and/or the washing reagent, and/or ranges from about 10 mM up to about 100 mM. The binding reagent, filtering reagent and/or the washing reagent can have a pH ranging from about pH 6.0 to about pH 8.0 (such as from about 6.5 to about 7.5).

In some embodiments, the eluting reagent can have a pH greater than about 9, greater than about 10, greater than about 11, or greater than about 12. The use of high pH to elute nucleic acid such as DNA is unique especially to the cartridges described herein and provides improved speed and performance of the disclosed methods. Speed is provided by the rapid neutralization of acidic ammonium ions by the high concentration of hydroxide ions. Alkylamines have a pKa˜10-11 and are immediately deprotonated at pH 12.7, to form the neutral free base on the solid surface, and release the cationic DNA. A further advantage of the high pH is the denaturing effect of KOH on captured DNA or RNA. Acidic functional groups in the heterocyclic bases of DNA or RNA are immediately deprotonated and cannot form Watson-Crick bonds. Double-stranded structures and other secondary structures are disrupted, but can re-nature when neutralized for example, with Tris HCl. This chemical denaturing of captured genomic DNA can be an advantage for isothermal assays that do not undergo the usual heat denaturing of PCR. The cartridges provided herein allow for rapid neutralization of eluted DNA or RNA in KOH followed by reaction with Tris to produce a final pH of about 8.5 for downstream PCR or other nucleic acid assays. In some embodiments, the eluting reagent can have a pH less than about 9, less than about 8.5, or less than about 8. This lower-pH elution of bound DNA or RNA can be an advantage, especially for devices that don't facilitate rapid neutralization of the KOH solution. It is known that RNA is hydrolyzed by high pH, but short exposure times to KOH can provide for good quality RNA. In some examples, the eluting reagent comprises a polyanion, a polycation, ammonia or an alkali metal hydroxide. For example, the eluting reagent may comprise a polyanion such as a carrageenan, a carrier nucleic acid, or a combination thereof.

In some instances, to reduce bubble formation in one or more of the chambers, the detergent Brij may be added to one or more of the reagents described herein.

It is understood that various other reagents and initial volumes can be used for performing an automated PCR panel assay on a sample inserted into the cartridge.

### Specialized Cartridge Components

FIG. 6 illustrates an exemplary cartridge suitable for performing a multi-target panel assay, as described herein. The illustrated cartridges are based on the GENEXPERT® cartridge (Cepheid, Inc., Sunnyvale, Calif.). The cartridge 100 comprises a cartridge body 102 having multiple chambers 108 defined therein for holding various reagent and/or buffers. The chambers are disposed around a central syringe barrel 109 that is in fluid communication with valve body 110 through valve syringe tube 106 extending through the syringe barrel 109. In some embodiments, the valve assembly include any of the valve assemblies described herein, or any suitable valve assembly. In this embodiment directed to an cartridge, the cartridge utilizes a valve assembly capable of chemical lysing, for example, any of valve assemblies B-D in FIG. 2. The valve body 110 is interface within the cartridge body and supported on a cartridge base 101. The cartridge typically contains one or channels or cavities that can contain a filter material (e.g. glass filter column) as described herein that can function to bind and elute a nucleic acid. In various embodiments, the cartridge further comprises one or more temperature controlled channels or chambers that can, in certain embodiments, function as thermocycling chambers. A “plunger” not shown can be operated to draw fluid into the syringe barrel 109 and rotation of the valve body/syringe tube provides selective fluid communication between the various reagent chambers and channels, reaction chamber(s). Thus, the various reagent chambers, reaction chambers, matrix material(s), and channels are selectively in fluid communication by rotation of the valve and plunger and reagent movement (e.g., chamber loading or unloading) is operated by the “syringe” action of the plunger. The attached reaction vessel 116 provides optical windows to provide real-time detection of, e.g., amplification products, base identity in sequencing operations, by operation of the module within the system described herein.

While the methods described above are described with respect to specific chambers in the GENEXPERT® cartridge, it will be recognized that the particular reagent/chamber assignments can be varied depending on the particularities of the nucleic acid detection/quantification assay. It will also be recognized that in certain embodiments, variants of the GENEXPERT® cartridge are also contemplated. Such variants can include, but are not limited to, more reagent chambers or fewer reagent chambers and/or different sized chambers, two (or more) sample receiving chambers, two (or more) temperature controlled channels or chambers, stacked cartridges (providing control of two cartridges by one module), and the like. In one aspect, the sample cartridge includes one or more features or components that are specially configured per the unique requirements of a particular multi-target assay. In this embodiment, the sample cartridge utilizes certain components specifically developed for the Respiratory Panel assay.

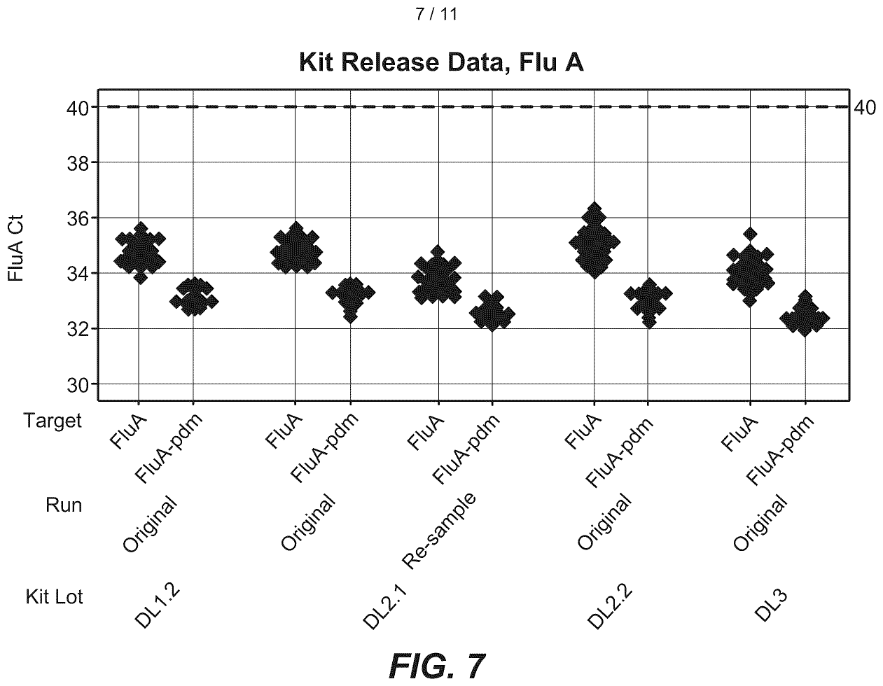

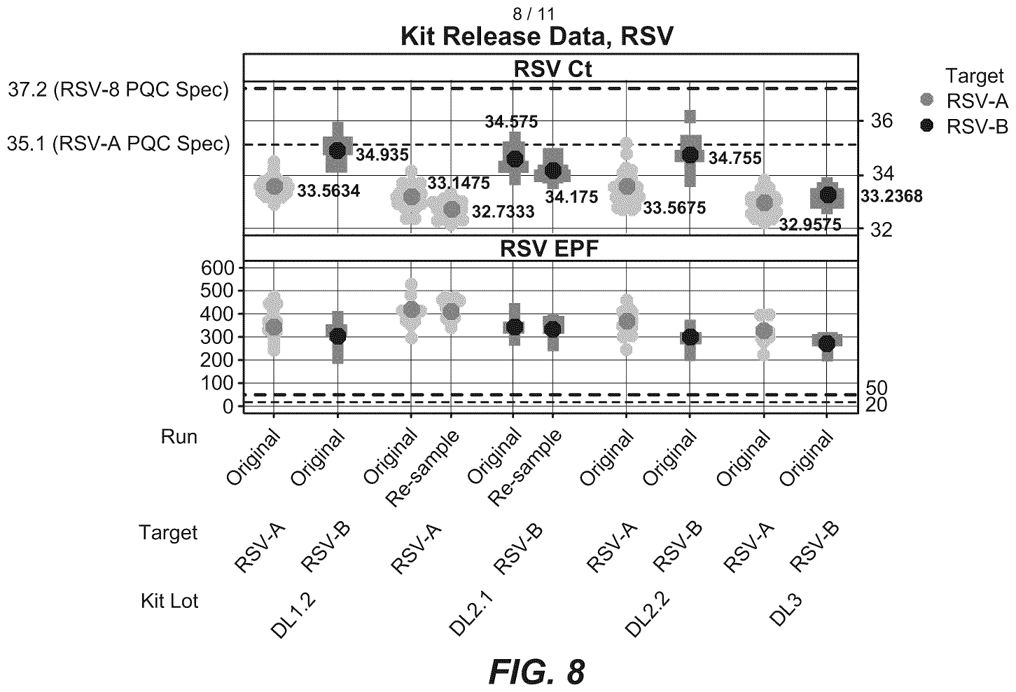

FIGS. 8A-8B show a detailed view of a valve assembly for use in a multi-target panel assay cartridge, in accordance with some embodiments. In this embodiment, the valve assembly is configured for chemical lysing. The valve assembly includes a valve body 110 having a disk shape and tubular syringe type 106 along a proximal portion thereof. The disk portion has a generally planar external upper port surface 120 having one or more ports therein (e.g., 142, 146), as shown in FIG. 7A. The valve assembly is rotatable relative to the cartridge housing, for example by engagement of an interface feature 123 with a driven mechanism of the module. The ports 142, 146 on the top planar surface permit fluid communication between the syringe tube 106 and each of the chambers within the cartridge and/or one or more channels. In this embodiment, the ports 142, 146 interface with a circular gasket 126 having multiple ports defined therein which interfaces and fluidically couple with corresponding ports on the bottom of the chambers in the cartridge body. Upon rotation of the valve assembly, the top ports 142, 146 interface with differing pairs of ports so as to fluidically couple the various chambers to each other or to channels of the reaction vessel attached to the cartridge so that movement of the “plunger” through the syringe tube advances fluid between the various chambers and/or channels to facilitate processing and subsequent analytical testing of the fluid sample.

As seen in FIG. 7B, the valve body 110 includes a lysing chamber 121 on an underside thereof that may contain solid phase material for capturing cells, spores, viruses, or microorganisms to be lysed. Suitable solid phase materials include, without limitation, filters, beads, fibers, membranes, filter paper, glass wool, polymers, or gels. One or more interior ports facilitate fluid flow into and out of the lysing chamber. In this embodiment, the solid phase material is a glass filter 114 that resides in a filter recess 111 in the valve body 110 for capturing nucleic acid released by chemical lysing of the target organisms in the fluid sample.

As shown in FIGS. 8A-8B, an outer wall 122 of the valve body encloses the lysing chamber 121 along with bottom cap 112. As seen in FIG. 7B, the lysing chamber 121 includes a pair of processing ports to facilitate flow of fluid sample into and out of the lysing chamber. A first fluid processing port 124 coupled to a first fluid processing channel defined within the valve body, and a second fluid processing port 125 coupled to a second fluid processing channel defined within the valve body. Through these channels, the first fluid processing channel is coupled to both the inner conduit of the syringe tube 106 and external port 142, while the second fluid processing channel is coupled to the second external port 146. By this configuration, application of pressure by movement of a “plunger” through the syringe tube 106 acts like a syringe, drawing fluid from one chamber into the syringe tube, and after rotation of the valve assembly to position the respective ports to couple with another chamber, movement of the plunger advances the fluid through the valve body and channels into another chamber or channel to the reaction vessel. This approach can perform the various processing steps required to perform a sample preparation protocol for the targeted analytes of the multi-target panel assay.

In one aspect, the respiratory panel described herein performs chemical lysing of the targeted bacteria and viruses. Often, in conventional sample cartridges and methods, these bacteria targets are mechanically lysed (e.g., by ultrasonic lysing), whereas chemical lysing is usually reserved for less robust targets such as viruses. In order to perform chemical lysing of both bacterial and viral targets, lysing buffers may have elevated alkalinity (e.g., sodium hydroxide). Further high alkalinity eluting buffers (e.g., ammonia or an alkali metal hydroxide) may be used to elute the nucleic acids bound to the glass filter. While these buffers allow for chemical preparation of the sample, in practice, use of such buffers can be problematic in conventional cartridge as these high alkalinity buffers can degrade the valve assembly material and sealing interfaces between cartridge components, resulting in cracking of the valve assembly and leakage during processing. Such leakage can be detrimental to sample processing. A specialized valve assembly can be used to resist elevated alkalinity (e.g., greater than pH of 10, greater than pH 11, or greater than pH 12) of these buffers. One difficulty in developing conventional valve assemblies is that these valve assemblies are fabricated by injection molding of certain polymer materials, typically polycarbonate, polyolefin (including polyethylene or polypropylene), or combinations thereof. Typical such polymer materials used in injection molding are not resistant to the range of elevated alkalinity noted, and polymer materials that are resistant, may be considerably more costly and may be less suited for injection molding of small-scale microfluidic features. Thus, the specialized valve assemblies can be developed by annealing valve body assemblies formed with conventional polymers in order to harden the polymer material sufficiently to resist elevated alkalinity. Annealing of polycarbonate reduces chemical corrosion by the mixture of NaOH and GTC within the lysis reagent and the eluting reagent. In some embodiments, after the valve assemblies are formed, they are heated to an elevated temperature (e.g., 90-100° C., about 100° C.) for about an hour or more, then allowed to slowly cool in a temperature-controlled manner for at least 30 minutes. Studies showed that these annealed valve assemblies resulted in valve assemblies of substantially the same design and material as conventional valve assemblies, except they were resistant to high alkalinity buffers that could not have been feasible to use in conventional cartridges. Testing of the annealed valve body to alkaline resistance can be performed by exposing the valve body assembly (VBA) to NaOH/GTC, followed by visual inspection for cracking of VBA after 30 min or so. It is appreciated that this annealed cartridge can be advantageous for various reasons and need not be tied to any particular assay described herein. Moreover, it is further appreciated that the assays described herein may be performed with various other cartridge designs, devices and systems and need not be tied to the particular cartridge designs described herein.

### Exemplary Detection Methods, Results, and Handling of Results

In some embodiments, a computer-based analysis program is used to translate the raw data generated by the detection assay into data of predictive value for a clinician. Analyte names in respect to the pathogens and detection methods, in an illustrative, automated embodiment, are listed in Table C.

TABLE C
Respiratory Biomarker Panel Targets

| Target | Analyte Name | Detection Method |
|---|---|---|
| Adenovirus | Adenovirus | Amplification |
| Coronavirus | Coronavirus | Amplification |
| SARS-CoV-2 | SARS-CoV-2 | Amplification |
| Influenza A | Flu A | Amplification |
| Influenza A H1N1 09 | Flu A H1N1- 09 | Melt |
| Influenza B | Flu B | Amplification |
| Human Metapneumovirus | hMPV | Melt |
| Parainfluenza virus | Parainfluenza | Amplification |
| Rhinovirus/Enterovirus | Rhino-Enterovirus | Amplification and |
| | | Melt |
| Respiratory Syncytial Virus | RSV | Amplification |
| Bordetella parapertussis | B. parapertussis | Melt |
| Bordetella pertussis | B. pertussis | Melt |
| Chlamydia pneumoniae | C. pneumoniae | Melt |
| Mycoplasma pneumoniae | M. pneumoniae | Melt |

Melt Curve analysis is evaluated by GENEXPERT® Software to determine the presence of PCR product. Melting temperature (T_{M}) and Melt peak height of the curve is calculated automatically by the analysis software. The melt curve is detected as positive if Tₘ falls inside the valid Tₘ range specified for each target nucleic acid. The melt curve is called as negative if melt curve is not in the appropriate Tₘ range. The following pathogens can be detected using amplification curve analysis (cycle threshold): Influenza B virus, Coronavirus SARS-COV-2, Coronavirus, Adenovirus, Rhinovirus/Enterovirus, Respiratory Syncytial Virus, Parainfluenza virus and Influenza A. The software automatically calculates the cycle threshold (Ct), Endpoint and Probe check values. Illustrative limit-of-detection concentrations, Ct cut-offs, and methods for determining the same are provided in the examples below.

Before the start of the PCR reaction, the GENEXPERT® System measures the fluorescence signal from the probes to monitor bead rehydration, reaction tube filling, probe integrity, and dye stability. This Probe Check Control (PCC) passes if it meets validated acceptance criteria.

In some embodiments, a computer-based analysis program is used to translate the raw data generated by the detection assay into data of predictive value for a clinician. The clinician can access the predictive data using any suitable means. Thus, in some embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

When the GENEXPERT® System is used, the results are interpreted automatically and are shown in a “View Results” window. Positive targets, negative targets, and indeterminate targets are presented in different colors. Samples with coinfection may appear with positives results for multiple targets. Invalid, Error or No result are also presented.

A positive result generally indicate the target has a Ct within the valid range and endpoint above the threshold setting and/or the target has a Melt Peak above the threshold setting and melt temperature (Tₘ) window within the valid range; SPC can be ignored when amplification occurred; all probe check results pass; and except for the target indicated as positive, all other pathogens listed in Table 1 are not detected.

### Exemplary Detection Methods for One to Forty Targets

As described herein, the respiratory (or other viral, bacterial, fungal, or parasitic) pathogens can be detected by nucleic acid amplification in a multiplex amplification reactions, which can be designed to detect from 1 up to 40 (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39) or more target nucleic acids per amplification reaction mixture. Methods for identifying up to forty target nucleic acids in a biological sample collected from a subject are disclosed herein. The method for identifying up to forty target nucleic acids in a biological sample collected from a subject can include providing a sample cartridge as described herein. The sample cartridge preferably comprises a plurality of chambers including a lysis chamber having at least a fluid outlet in fluid communication with another chamber of the plurality, the lysis chamber adapted for performing mechanical and chemical lysis to release nucleic acid from the biological sample. The sample cartridge also preferably comprises lyophilized and liquid reagents in two or more of the plurality of chambers, the lyophilized reagents comprising at least one primer set for amplifying and detecting the target nucleic acid(s), and the liquid reagent including a lysis buffer, a binding reagent, and an eluting buffer. The sample cartridge further preferably comprises a reaction vessel fluidically coupled to the plurality of chambers of the sample cartridge and configured for performing amplification of the target nucleic acids.

The method for identifying up to forty target nucleic acids in a biological sample collected from a subject further includes contacting nucleic acid from the biological sample with the at least one primer set. The number of primer sets present depends on the number of target nucleic acid the assay identifies. For example, the sample cartridge can include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more primer sets. Each primer set comprises a forward primer, a reverse primer, and a probe specific for a target nucleic acid in the biological sample. As described herein, the primer set(s) can be present in the sample cartridge as lyophilized beads. More than one primer set can be present in each bead. For example, each lyophilized bead can include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more of the primer sets. The probe in each set can comprise a detectable moiety for detection via real-time PCR or melt curve analysis

Within each primer set, at least one of the forward primer or the reverse primer can comprise one or more modified bases. The modified base can be selected from a destabilizing base, a stabilizing base, or a combination thereof. “Stabilizing bases” include, e.g., stretches of peptide nucleic acids (PNAs) that can be incorporated into DNA oligonucleotides to increase duplex stability. Locked nucleic acids (LNAs) and unlocked nucleic acids (UNAs) are analogues of RNA that can be easily incorporated into DNA oligonucleotides during solid-phase oligonucleotide synthesis, and respectively increase and decrease duplex stability. Suitable stabilizing bases also include modified DNA bases that increase the stability of base pairs (and therefore the duplex as a whole). These modified bases can be incorporated into oligonucleotides during solid-phase synthesis and offer a more predictable method of increasing DNA duplex stability. Examples include AP-dC (G-clamp) and 2-aminoadenine, as well as 5-methylcytosine and C(5)-propynylcytosine (replacing cytosine), and C(5)-propynyluracil (replacing thymine). Stabilizing bases can increase melting temperature of the primer in double stranded form or reduce secondary structure formation. “Destabilizing bases” are those that destabilize double-stranded DNA by virtue of forming less stable base pairs than the typical A-T and/or G-C base pairs. Destabilizing bases can be selected from a bulky base to reduce unspecific primer-primer interactions, a base that suppresses melting temperature of the primer in double-stranded form, a base that suppresses extension of unwanted primer-dimer, or a combination thereof. Inosine (I) is a destabilizing base because it pairs with cytosine (C), but an I-C base pair is less stable than a G-C base pair. This lower stability results from the fact that inosine is a purine that can make only two hydrogen bonds, compared to the three hydrogen bonds of a G-C base pair. Other destabilizing bases are known to, or readily identified by, those of skill in the art.

In some examples, the primer and/or probe includes a modified base that reduces unwanted hybridization. Unwanted hybridization between any two oligonucleotides in an assay can be reduced or prevented by including pseudo-complementary bases in the oligonucleotides. Pseudo-complementary bases are described as “modified bases” as described herein. Modified bases useful in the primers and other oligonucleotides described herein include those wherein the modified base forms stable hydrogen-bonded base pairs with the natural complementary base but does not form stable hydrogen-bonded base pairs with its modified complementary base (e.g., pseudo-complementary bases). (For ease of discussion, complementary bases are also referred to herein as “partners.”) In some embodiments, this is accomplished when the modified base can form two or more hydrogen bonds with its natural partner, but only one or no hydrogen bonds with its modified partner. This allows the production of primer and other oligonucleotide pairs that do not form substantially stable hydrogen-bonded hybrids with one another, as manifested in a melting temperature (under physiological or substantially physiological conditions) of less than about 40° C. The primers of the primer pair, however, form substantially stable hybrids with the complementary nucleotide sequence in a template strand (e.g., first template strand) of a single- or double-stranded target nucleic acid and with a strand complementary to the template strand (e.g., second template strand). In some embodiments, due to the increased (in some embodiments, double) number of hydrogen bonds in such hybrids, the hybrids formed with the primers of the present invention are more stable than hybrids that would be formed using primers with unmodified bases. Pseudo-complementary bases are described in U.S. Patent Publication No. 2023/0167490A1, the content of which is incorporated herein by reference for this description.

In general, a sufficient number of modified nucleotides are incorporated into the primers described herein to preferentially increase the annealing of the primers to the template strands of a target nucleic acid, as compared to primer-to-primer annealing. It is not necessary to replace each natural nucleotide of the primer with a modified nucleotide in order to accomplish this. In some embodiments, the primers include, in addition to one or more modified nucleotides, one or more naturally occurring nucleotides and/or variants of naturally occurring nucleotides, provided that the variations do not interfere significantly with the complementary binding ability of the primers, as discussed above. For example, primers including modified nucleotides can include pentofuranose moieties other than ribose or 2-deoxyribose, as well as derivatives of ribose and 2-deoxyribose, for example 3-amino-2-deoxyribose, 2-fluoro-2-deoxyribose, and 2-O—C₁₋₆ alkyl or 2-O-allyl ribose, particularly 2-O-methyl ribose. The glycosidic linkage can be in the α or β configuration. The phosphate backbone of the primer can, if desired, include phosphorothioate linkages.

A general structure for a suitable class of the modified A analog, A*, shown as a 3′-phosphate (or phosphorothioate) incorporated into a primer, is provided by Formulas 1, 2, and 3, below, wherein: X is N or CH; Y is O or S; Z is OH or CH₃; R is H, F, or OR₂, where R₂ is C₁₋₆alkyl or allyl, or H in case of RNA; and R₁ is C₁₋₄alkyl, C₁₋₄ alkoxy, alkylthio, F, or NHR₃, where R₃ is H, or C₁₋₄ alkyl. An illustrative embodiment of A* has 2,6-diaminopurine (2-aminoadenine) as the base. A general structure for a suitable class of the modified T analog, T*, shown as a 3′-phosphate (or phosphorothioate) incorporated into the primer, is provided by Formula 4, wherein: Y, Z, and R are defined as above; and R₄ is H. C₁₋₆ alkyl, C₁₋₆ alkenyl, or C₁₋₆ alkynyl. An illustrative embodiment of T* has 2-thio-4-oxo-5-methylpyrimidine (2-thiothymine) as the base. The latter nucleotide can be abbreviated as 2-sT or d2-sT, as applicable. A general structure for a suitable class of the modified G analog, G*, shown as a 3-phosphate (or phosphorothioate) incorporated into the primer, is provided by Formulas 9, 10 and 11, wherein: R₁ is H, C₁₋₄ alkyl, C₁₋₄ alkoxy, C₁₋₄ alkylthio, F, or NHR₃, where Ra is defined as above; and X, Y, Z, and R are defined as above. An illustrative embodiment of G* has 6-oxo-purine (hypoxanthine) as the base. A general structure for a suitable class of the modified C analog, C*, shown as a 3′-phosphate (or phosphorothioate) incorporated into the primer, is provided by Formula 8, wherein: Y, Z, R, and R₄ are defined as above; Z₁ is O or NH; and R₅ is H or C₁₋₄ alkyl. An illustrative embodiment of C* has pyrrolo-[2,3-d]pyrimidine-2(3H)-one as the base. The above-described modified bases and nucleotides are also described in U.S. Pat. No. 5,912,340 (issued Jun. 15, 1999 to Kutyavin et al.), which is hereby incorporated by reference for this description. The hybridization properties of d2-amA and d2-sT are described in Kutyavin, et al. (1996) Biochemistry 35:11170-76, which is also hereby incorporated by reference for this description. The synthesis and hybridization properties of d/I and dP are described in Woo et al. (1996) Nucleic Acids Research 25 (13): 2470-75, which is also hereby incorporated by reference for this description. Additional examples of G* and C* include 7-alkyl-7-deazaguanine and N⁴-alkylcytosine (where the alkyl is methyl or ethyl), respectively, which are described in Lahoud et al. (2008) Nucleic Acids Research 36 (10): 3409-19 (hereby incorporated by reference for this description). Further examples of G* and C* include 7-nitro-7-deazahypoxanthine (NitrocH) and 2-thiocytosine (sC), respectively, which are described in Lahoud et al. (2008) Nucleic Acids Research 36 (22): 6999-7008 (hereby incorporated by reference for this description). Hoshinka et al. (2010) Angew Chem Int Ed Engl. 49 (32): 5554-5557 describes the use of such bases (“Self-Avoiding Molecular Recognition Systems”), including 2′-hypoxanthine as G+ (this reference is hereby incorporated by reference for this description; see especially, FIG. 1); see also Yang et al. (2015) Chembiochem. 16 (9): 1365-1367 (this reference is hereby incorporated by reference for this description; see especially, Scheme 1).

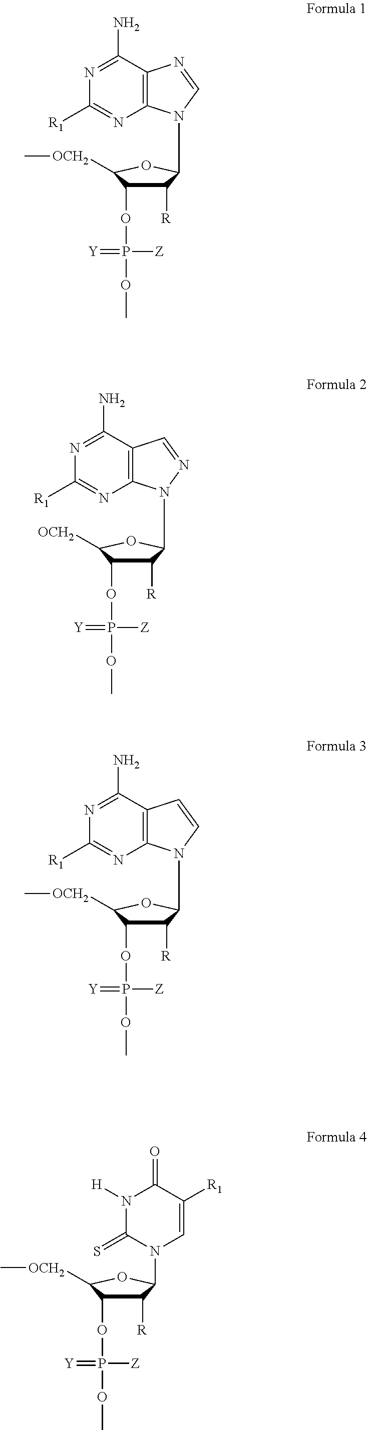

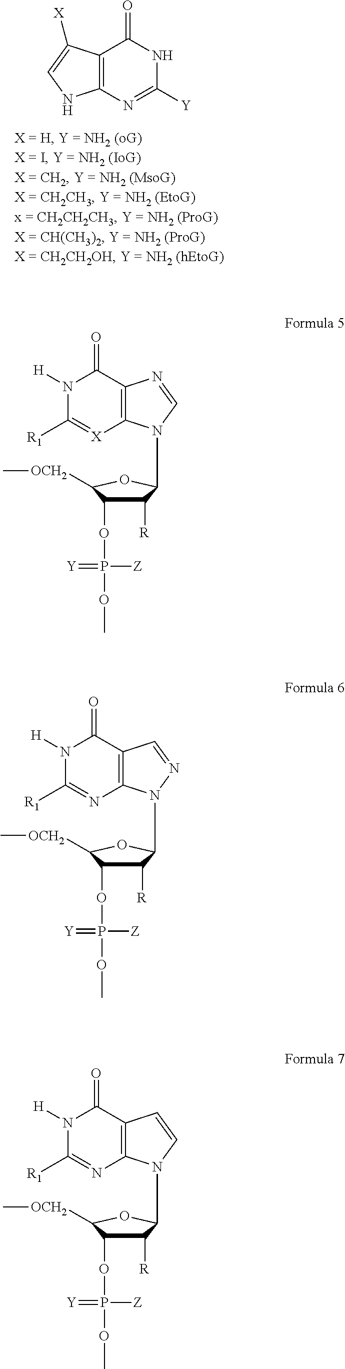

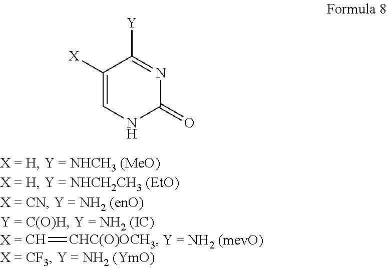

In the methods described herein for identifying up to forty (or more) target nucleic acids in a biological sample collected from a subject, each of the forward primer, reverse primer, and probe within a primer set for a target nucleic acid has less than 10% homogeneity with a forward primer, a reverse primer, and a probe within a different primer set.

The methods for identifying up to forty (or more) target nucleic acids in a biological sample collected from a subject further include subjecting the nucleic acid, primers, and probes within the reaction vessel to amplification conditions, optionally followed by a melt curve assay, and detecting the presence of any amplification product(s) via real-time PCR and optionally melt curve analysis, by an optical assembly. Multi-channel optical detection assemblies are described in U.S. Pat. No. 6,369,893B1, embodiments of which are incorporated herein by reference. The optical assembly utilized in the current disclosure preferably comprise at least ten optical channels configured to simultaneously transmit excitation beams in at least ten excitation wavelength ranges, and simultaneously detect emitted light in at least ten emission wavelength ranges to and from the reaction vessel. Since the optical assembly can be limited by the number of wavelengths emitted or detected, when greater than 10 target nucleic acids are to be identified, one more probes for detection via melt curve analysis and one or more probes for detection via real-time PCR can be combined in a single optical channel. In some embodiments, the optical assembly simultaneously detects up to ten (10) emission wavelength ranges via real-time PCR in each amplification cycle and up to ten (10) emission wavelength range in each melt curve cycle, in each reaction chamber. In certain embodiments, method differentially identifies up to thirty (30) target nucleic acids in each melt curve assay, by combining probes for a plurality (up to three) of target nucleic acids in each optical channel and defining melt windows for each target nucleic acid. When two or more probes are combined in an optical channel, probe(s) detected by melt curve assay are configured to have a Tm below annealing temperature (no amplification curve). Some channels, however, have a plurality of probes (up to 4 probes) but they are all amplification probes or are all melt probes. When greater than ten (10) target nucleic acids are to be identified, target nucleic acids from viruses can be detected via real-time PCR, and target nucleic acids from bacteria can be detected via melt curve assay. There are exceptions, however, where viruses are detected by melt and bacteria by real-time PCR.

The method for identifying up to forty target nucleic acids in a biological sample collected from a subject further include differentially identifying the presence of the target nucleic acid(s) in the sample or determining that no target nucleic acid is detectable based on detection of the amplification product(s) or lack thereof, respectively.

The lysis chamber together with the lysis reagent used in the methods described herein are adapted to lyse viral, bacterial, fungal, protozoan, and epithelial cells present in the biological sample. In some aspects, the method does not include mechanical lysis of the biological sample. For example, the methods can utilize chemical lysis only. In other aspects, the method includes mechanical lysis of the biological sample. The biological sample can be selected from a respiratory sample (e.g., nasopharyngeal swab (NP), oral-pharyngeal swab (OP), nasal swab (NS), respiratory mucus sample, respiratory tissue sample, respiratory cell sample), saliva sample, sputum sample, blood, plasma, serum, semen, spinal fluid, tissue, tear, urine, stool, smear preparation, vaginal swab, vaginal mucus sample, vaginal tissue sample, vaginal cell sample, bacterial culture, mammalian cell culture, viral culture, human cell, bacteria, extracellular fluid, pancreatic fluid, cell lysate, PCR reaction mixture, in vitro nucleic acid modification reaction mixture, or combination thereof. In some examples, the target nucleic acids are selected from a viral pathogen, a bacterial pathogen, a fungal pathogen, a protozoan pathogen, or a combination thereof. For example, the target nucleic acids can be selected from a viral respiratory pathogen, a bacterial respiratory pathogen, or a combination thereof.

Sample cartridges for identifying up to forty target nucleic acids in a biological sample collected from a subject are also disclosed. The sample cartridge can comprise a cartridge body comprising a plurality of chambers therein. As described herein, the plurality of chambers includes a sample chamber having at least a fluid outlet in fluid communication with another chamber of the plurality; a lysis chamber in fluidic communication with the sample chamber, wherein the lysis chamber is adapted for performing mechanical and/or chemical lysis to release nucleic acid from the biological sample, optionally wherein the sample chamber and lysis chamber are the same; a lyophilized reagent chamber comprising a lyophilized PCR master mix including at least one primer set for amplifying and detecting the target nucleic acids, and one or more liquid reagent chambers comprising a lysis buffer, a binding reagent, and an eluting buffer. The sample cartridge also comprises a reaction vessel fluidically coupled to the plurality of chambers of the cartridge body and configured for amplification of nucleic acid and melt curve assay and adapted for detection of one or a plurality of amplification products via real-time PCR and melt curve analysis by an optical assembly. In some embodiments, the reaction vessel can comprise up to 5 reaction chambers, each reaction chamber configured for performing amplification of the up to forty target nucleic acids in solution. In some embodiments, the reaction vessel comprises a single reaction chamber. The optical assembly comprises at least ten optical channels configured to simultaneously transmit excitation beams in at least ten excitation wavelength ranges to the reaction vessel, and simultaneously detect emitted light in at least ten emission wavelength ranges from the reaction vessel. When greater than 10 target nucleic acids are to be identified, one or more probes for detection via melt curve analysis and one or more probes for detection via real-time PCR are combined in a single optical channel. The sample cartridge further comprises a filter disposed in a fluidic path between the lysis chamber, if present, or the sample chamber, and the reaction vessel.

The sample cartridge can be further adapted for performing high-resolution melt detection, nested PCR, multiphasic detection, or a combination thereof. In some embodiments, the methods disclosed herein do not include nested PCR.

Diagnostic assay systems for identifying up to forty target nucleic acids in a biological sample collected from a subject are also disclosed. The systems can comprise a sample cartridge as described herein, a thermal assembly configured to subject the nucleic acid, primers, and probes within the reaction vessel of the sample chamber to amplification conditions, optionally followed by a melt curve assay; an optical assembly comprising at least ten optical channels and configured to simultaneously transmit excitation beams in at least ten excitation wavelength ranges, and simultaneously detect emitted light in at least ten emission wavelength ranges to and from the reaction vessel, respectively; an electronic device for (i) receiving and processing signals detected due to the presence of any amplification product(s) via real-time PCR and melt curve assay, by the optical assembly; (ii) differentially identifying the presence of a target nucleic acid in the sample, or determining that no target nucleic acid is detectable based on detection of the amplification product(s), melt curve, or lack thereof; and (iii) outputting a real-time PCR result for the sample in the reaction vessel during or after the amplification, and outputting a melting result for the sample in the reaction vessel during or after the melt probe assay; and a communication interface in communication with an electronic device that comprises a display screen having a user interface that displays one or more graphical elements that permit a user to input a selection corresponding to at least one target assay from a plurality of target assays, which at least one target assay is directed to determining presence, absence or amount of at least one target nucleic acid in the biological sample. In some aspects, the system is configured to tune the number of amplification cycles to the titers of the target nucleic acids. The respiratory panel can be a qualitative test in which positive or negative call-outs are made. All targets are preferably tested at high concentration to make sure they are detected.

The present disclosure contemplates any method capable of receiving, processing, and transmitting the information to and from laboratories conducting the assays, information provides, medical personal, and subjects. For example, in some embodiments of the present invention, a sample is obtained from a subject and submitted to a testing service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample collected and sent to the testing service, or subjects may collect the sample themselves and directly send it to a testing service. Where the sample includes previously determined biological information, the information may be directly sent to the testing service by the subject (e.g., an information card containing the information may be scanned by a computer and the data transmitted to a computer of the profiling center using an electronic communication systems). Once received by the testing service, the sample is processed and a set of test results is produced, specific for the diagnostic or prognostic information desired for the subject.

The test results can be prepared in a format suitable for interpretation by a treating clinician. For example, rather than providing raw expression data, the prepared format may represent a diagnosis or risk assessment for the subject, with or without recommendations for particular treatment options. The test results may be displayed to the clinician by any suitable method. For example, in some embodiments, the testing service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may choose further intervention or counseling based on the results. In some embodiments, the data is used for research use. For example, the data may be used to further optimize the inclusion or elimination of markers as useful indicators of a particular condition or stage of disease or as a companion diagnostic to determine a treatment course of action.

### Kits

Also contemplated is a kit for carrying out the methods described herein. Such kits include one or more reagents useful for practicing any of these methods. A kit generally includes a package with one or more containers holding the reagents, as one or more separate compositions or, optionally, as an admixture where the compatibility of the reagents will allow. The kit can also include other material(s) that may be desirable from a user standpoint, such as a buffer(s), a diluent(s), a standard(s), and/or any other material useful in sample processing, washing, or conducting any other step of the assay.

Kits preferably include instructions for carrying out one or more of the screening methods described herein. Instructions included in kits can be affixed to packaging material or can be included as a package insert. While the instructions are typically written or printed materials they are not limited to such. Any medium capable of storing such instructions and communicating them to an end user can be employed. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

In some embodiments, a kit includes primer pairs for amplifying and/or detecting the above-described respiratory pathogen biomarker panel targets described above, optionally with probes specific for these targets. In some embodiments, these kits can include primers pairs and optional probes for detecting one or more of the above-described controls

In some embodiments, the kit can include any the reagents described above provided with or in one or more GENEXPERT® cartridge(s). See e.g., U.S. Pat. Nos. 5,958,349, 6,403,037, 6,440,725, 6,783,736, 6,818, 185; each of which is herein incorporated by reference for this description).

Any of the kits described here can include, in some embodiments, a receptacle for a nasal aspirate/wash sample and/or a swab for collecting a nasopharyngeal swab sample.

### EXAMPLES

### Example 1: Respiratory Panel Limit of Detection (LOD) Study Report

This example shows the output of a Respiratory Panel Limit of Detection (LOD) Study. Data collected was used to verify the analytical performance of the Respiratory Panel test.

### Abbreviations

ADF—Assay Definition File; CCU—Color Changing Unit; CEID—Chicken Embryo Infectious Dose; CFU—Colony Forming Unit; CI—Confidence Interval; CLSI—Clinical and Laboratory Standards Institute; Cp—Copy; Ct—Cycle Threshold; CV—Coefficient of Variation; EDTA—Ethylenediaminetetraacetic Acid; FDA—Food and Drug Administration; Flu—Influenza; GX—GeneXpert®; hMPV—Human metapneumovirus; IRR—International Reagent Resource; IVD—In vitro diagnostics; LOD—Limit of Detection; LSP—Lot Specific Parameters; NIBSC—National Institute for Biological Standards and Control; NP—Nasopharyngeal; PFU—Plaque Forming Unit; RNA—Ribonucleic Acid; RSV—Respiratory Syncytial Virus; SD—Standard Deviation; TCID—Tissue Culture Infectious Dose; UTM—Universal Transport Media; WHO—World Health Organization

### SUMMARY

A study to determine the analytical sensitivity, or Limit of Detection (LOD), for each organism target included in the Respiratory Panel test was performed. The LOD was estimated by probit analysis and subsequently verified for each organism target. All Respiratory Panel organisms, strains, and the level of LOD verification are presented in Table 1. The Xpert Respiratory Panel acceptance criteria for LOD were met.

TABLE 1
Respiratory panel organisms, strains and the verified LOD

| Organism/Type/Strain | Verified LOD | Unit |
|---|---|---|
| Adenovirus B, Wan | 25 | TCID50/mL |
| Adenovirus 1C | 20 | TCID50/mL |
| Adenovirus 4(E) | 3.75 | TCID50/mL |
| Coronavirus 229E | 0.063 | TCID50/mL |
| Coronavirus HKU1 | 100 | Cp/mL* |
| Coronavirus NL63 | 0.05 | TCID50/mL |
| Coronavirus OC43 | 0.08 | TCID50/mL |
| SARS-CoV-2 WHO 1^{st} International Standard (NIBSC 20/146) | 1492 | IU/mL |
| SARS-CoV-2 Hong Kong/VM20001061/2020 | 2 | TCID50/mL |
| Human metapneumovirus (hMPV) 18 B2, IA18-2003 | 0.5 | TCID50/mL |
| Human Rhinovirus 3, FEB | 4 (3) | TCID50/mL |
| | | (PFU/mL) |
| Enterovirus D68, US/MO/14-18947 | 8 | TCID50/mL |
| Parainfluenza 1, C35 | 8 | TCID50/mL |
| Parainfluenza 2, Greer | 2 | TCID50/mL |
| Parainfluenza 3, C 243 | 10 | TCID50/mL |
| Parainfluenza 4A | 8 | TCID50/mL |
| Influenza A/Wisconsin/04/2018 (H3N2) | 16 | CEID50/mL |
| Influenza A/Wisconsin/505/2018 (H1N1)pdm09 | 0.5 | TCID50/mL |
| Influenza B/Missouri/12/2018 (NA D197E) (Victoria Lineage) | 4 (1) | TCID50/mL |
| | | (Cp/mL) |
| Influenza B/Indiana/17/2017 (NA I221T) (Yamagata Lineage) | 4.4 (1.5) | TCID50/mL |
| | | (Cp/mL) |
| Respiratory Syncytial Virus (RSV) A | 0.1 | TCID50/mL |
| Respiratory Syncytial Virus (RSV) B, WV/14617/85 | 0.025 | TCID50/mL |
| Bordetella Pertussis, A639 | 200 | CFU/mL |
| Bordetella Parapertussis, A747 | 8 | CFU/mL |
| Mycoplasma pneumoniae, M129 | 15 (93) | CCU/mL |
| | | (Cp/mL) |
| Chlamydia pneumoniae, CM-1 | 1 | TCID50/mL |

*Titer available in cp/mL for coronavirus HKU1 clinical sample.

### Objective

To determine the analytical sensitivity, or Limit of Detection (LOD) of the Respiratory Panel test. The LOD is defined as the lowest concentration of an analyte that can be consistently detected in ≥95% of samples tested under routine clinical laboratory conditions and in a defined type of sample.

### Materials and Methods

The study was performed according to an Xpert Respiratory Panel Limit of Detection (LOD) Study Protocol. The study was designed in alignment with the Clinical and Laboratory Standards Institute (CLSI) guideline, EP17-A2, 2012, but with exception of using a smaller number of replicates than recommended in the logistic regression analysis, in accordance with correspondence with the FDA (Q202166).

The LOD was estimated for each target organism using a minimum of one strain. In addition, strains representing adenovirus B, C and E, all known serotypes of coronavirus (NL63, HKU1 229E and OC43), parainfluenza subtype 1-4, two types of influenza A (H3N2 and H1N1) and two types of influenza B (Yamagata and Victoria lineages) were included in the study. The LOD estimation was performed by logistic regression analysis using the probit approach.

For LOD verification, the concentration with positive reported result greater than or equal to 95% was determined as the verified LOD.

### Samples

All virus and bacterial stocks were purchased from external vendors as titered cultures except for the Corona HKU1 strain which was a clinical sample with a titer determined from Droplet Digital PCR (ddPCR) and the SARS-COV-2 WHO 1st International Standard (NIBSC 20/146). Additionally, some purchased cultures were also titered by ddPCR to obtain a LOD in the unit cp/mL, needed for subsequential design verification studies. Dilution panels for each strain were prepared by spiking the virus or bacteria respectively into pooled negative clinical nasopharyngeal (NP) sample matrix at concentrations spanning the expected LOD. The dilution series were made with the aim of at least 3 levels resulting in positive hit rates within the range of 0.10 to 0.90 and at least one level exceeding 0.95. The hit rate is defined as the number of replicates with a “detected” outcome per the total number of replicates tested. In addition, one negative sample level was also included and consisted of clinical sample matrix without spiked virus or bacteria.

### Testing Procedure

The LOD for each organism strain was initially estimated by testing a minimum of five-member dilution panel, that spanned over the expected LOD, in clinical sample matrix. Each panel member was tested using two Xpert Respiratory Panel reagent lots, across a minimum of three days with a minimum of three replicates per reagent lot and day. In addition, a negative sample was tested across three days with three replicates per lot and day. The results from the negative replicates were used for Probit analysis for all tested organism strains. The LOD was estimated for each individual strain, reagent lot and both reagent lots combined by logistic regression analysis using the probit approach.

Based on the LOD estimation for each strain, the maximum observed level for LOD from the different reagent lots was selected for verification. Each organism strain was diluted to the selected LOD level for verification in negative clinical sample matrix. The LOD verification was performed by testing a minimum of 20 replicates using one Xpert Respiratory Panel reagent lot in accordance with CLSI EP17-A2 and with correspondence with the FDA (Q202166). If the verification was not successful, i.e., a positive reported result less than 95%, the procedure was repeated at a higher concentration. The concentration with a positive reported result greater than or equal to 95% (19 out of 20 replicates detected) was determined as the verified LOD.

For each day of testing, one external control (altering one out of two positives) was tested for the duration of the study.

### External Controls

Two different external positive controls were altered and tested on each day of study testing using the Xpert Respiratory Panel test. The two external controls consisted of target organism mixes diluted in serum protein matrix. The external control test results were considered valid if all target organisms included in the control mix were reported as “POSITIVE” and the organism targets not included were reported as “NEGATIVE”. The external control compositions together with expected valid results are presented in Table 2.

TABLE 2
Composition and requirements for external positive controls

| | | Test Results | Test Results |
|---|---|---|---|
| | | External Control | External Control |
| External Control | Organism | Sample Mix 1 | Sample Mix 2 |
| External Control | Bordetella pertussis | POSITIVE | NEGATIVE |
| Sample Mix 1 | Mycoplasma pneumoniae | POSITIVE | NEGATIVE |
| | Bordetella parapertussis | POSITIVE | NEGATIVE |
| | Coronavirus | POSITIVE | NEGATIVE |
| | Chlamydia pneumoniae | POSITIVE | NEGATIVE |
| | Adenovirus | POSITIVE | NEGATIVE |
| | Influenza A H3N2 | POSITIVE | POSITIVE* |
| External Control | Parainfluenza | NEGATIVE | POSITIVE |
| Sample Mix 2 | Human Metapneumovirus | NEGATIVE | POSITIVE |
| | SARS-CoV-2 | NEGATIVE | POSITIVE |
| | Influenza A H1N1 -09 | NEGATIVE | POSITIVE |
| | RSV | NEGATIVE | POSITIVE |
| | Influenza B | NEGATIVE | POSITIVE |
| | Rhino-Enterovirus | NEGATIVE | POSITIVE |

The pandemic Influenza, i.e. Influenza A H1N1-09 included in the external control sample Mix 2, will not only result in a positive report out for Influenza A H1N1, but will also result in a positive report out for Influenza A H3N2. Thus, a positive report out for Influenza A H3N2 for both external control sample mix 1 and 2 is considered a valid result.

### Instruments

All testing was performed on calibrated GeneXpert® instrument systems using validated software 6.4.

### Retest

If the initial Xpert Respiratory Panel test was reported as indeterminate (INVALID, ERROR, NO RESULT), a single retest was performed, provided enough sample was available for retesting. If upon retest the result was still indeterminate, it was reported as such.

### Data Analysis

The LOD for each target organism was estimated by logistic regression analysis using the probit analysis in Minitab® (version 19.2020.1). The hit rate for each dilution was converted to normal probability units and plotted versus their nominal concentrations and fitted by a regression model. The concentration of virus/bacteria that was detected with a hit rate equal to 95% as determined by logistic regression analysis was used to estimate the LOD. The LOD was calculated for each individual strain for each reagent lot and the two reagent lots combined. The maximum observed LOD, from analysis of different reagent lots, was selected for subsequent verification.

The LOD verification was performed by calculating the positivity rate for each target organism strain. For the LOD to be considered valid, each analyte needed to be consistently detected in ≥95% of the samples tested.

The expected analytical sensitivity/LOD for each target organism in the Xpert Respiratory Panel values are defined in Table 3.

TABLE 3
Xpert Respiratory expected LOD

| Target Organism | Expected LOD | |
|---|---|---|
| Adenovirus | <1000 | TCID50/mL |
| Coronavirus | <250 | TCID50/mL |
| SARS-CoV-2* | <250 | Cp/mL |
| Human metapneumovirus (hMPV) | <1000 | TCID50/mL |
| Rhinovirus/Enterovirus | <100 | TCID50/mL |
| Parainfluenza | <100 | TCID50/mL |
| Influenza A (FluA) | <1000 | TCID50/mL |
| Influenza A (Flu A) H1N1pdm | <1000 | TCID50/mL |
| Influenza B (Flu B) | <1000 | TCID50/mL |
| Respiratory Syncytial Virus (RSV) | <5 | TCID50/mL |
| Bordetella Pertussis | <300 | CFU/mL |
| Bordetella Parapertussis | <300 | CFU/mL |
| Mycoplasma pneumoniae | <100 | CCU/mL |
| Chlamydia pneumoniae | <100 | TCID50/mL |

*Provide LOD for SARS-CoV-2 using the First WHO International Standard (20/146)

### Results and Analysis

### LOD Verification

The estimated LOD by probit at 95% CI for each Xpert Respiratory Panel organism are presented in Table 4.

TABLE 4
Probit results (95% CI for each Xpert Respiratory panel target organism,
specified for each reagent lot and both reagent lots combined)

| | | LOD | | |
|---|---|---|---|---|
| | | Estimated by | | |
| Target Organism | | Probit 95% | 95% CI | |
| Strain | Kit Lot | CI | Lower-Upper | Unit |
| Adeno virus B | Lot 1 | 7.58 | 5.96-9.20 | TCID50/mL |
| | Lot 2 | 8.64 | 6.51-10.76 | |
| | Across both kit lots | 8.10 | 6.78-9.42 | |
| Adenovirus C | Lot 1 | 3.82 | 2.37-5.28 | TCID50/mL |
| | Lot 2 | 13.60 | 7.32-19.88 | |
| | Across both kit lots | 10.24 | 7.16-13.32 | |
| Adenovirus 4(E) | Lot 1 | 0.05 | 0.03-0.07 | TCID50/mL |
| | Lot 2 | 0.62 | 0.28-0.96 | |
| | Across both kit lots | 0.42 | 0.28-0.57 | |
| Coronavirus 229E | Lot 1 | 0.019 | 0.011-0.026 | TCID50/mL |
| | Lot 2 | 0.010 | 0.007-0.013 | |
| | Across both kit lots | 0.015 | 0.011-0.019 | |
| Coronavirus | Lot 1 | 44.90 | 31.55-58.24 | Cp/mL |
| HKU1 | Lot 2 | 29.25 | 18.84-39.67 | |
| | Across both kit lots | 39.19 | 29.99-48.39 | |
| | Lot 1 | 0.016 | 0.008-0.024 | TCID50/mL |
| Coronavirus | Lot 2 | NA* | NA* | |
| NL63 | Across both kit lots | 0.011 | 0.007-0.015 | |
| Coronavirus | Lot 1 | 0.033 | 0.020-0.046 | TCID50/mL |
| OC43 | Lot 2 | 0.039 | 0.027-0.051 | |
| | Across both kit lots | 0.037 | 0.028-0.046 | |
| SARS WHO | Lot 1) | 1917 | 1317-2517 | IU/mL |
| | Lot 2 | 790 | 537-1042 | |
| | Across both kit lots | 1489 | 1160-1819 | |
| SARS | Lot 1 | 0.64 | 0.45-0.80 | TCID50/mL |
| (Hong Kong) | Lot 2 | 0.63 | 0.40-0.85 | |
| | Across both kit lots | 0.64 | 0.49-0.79 | |
| hMPV | Lot 1 | 0.125 | 0.095-0.151 | TCID50/mL |
| | Lot 2 | 0.096 | 0.070-0.123 | |
| | Across both kit lots | 0.113 | 0.093-0.133 | |
| Rhinovirus | Lot 1 | 2.55 | 1.72-3.37 | TCID50/ml |
| | | (1.78) | (1.21-2.36) | (PFU/mL) |
| | Lot 2 | 1.60 | 1.14-2.05 | |
| | | (1.12) | (0.80-1.43) | |
| | Across both kit lots | 2.17 | 1.68-2.65 | |
| | | (1.52) | (1.18-1.86) | |
| Enterovirus | Lot 1 | 2.24 | 1.68-2.79 | TCID50/mL |
| | Lot 2 | 2.20 | 1.71-2.68 | |
| | Across both kit lots | 2.23 | 1.85-2.60 | |
| Parainfluenza 1 | Lot 1 | 3.55 | 2.54-4.56 | TCID50/mL |
| | Lot 2 | 1.66 | 1.06-2.26 | |
| | Across both kit lots | 2.96 | 2.30-3.62 | |
| Parainfluenza 2 | Lot 1 | 1.12 | 0.67-1.57 | TCID50/mL |
| | Lot 2 | 0.78 | 0.45-1.11 | |
| | Across both kit lots | 0.97 | 0.69-1.25 | |
| Parainfluenza 3 | Lot 1 | 2.92 | 1.85-4.00 | TCID50/mL |
| | Lot 2 | 2.60 | 1.57-3.63 | |
| | Across both kit lots | 2.78 | 2.02-3.53 | |
| Parainfluenza 4 | Lot 1 | 4.25 | 2.71-5.79 | TCID50/mL |
| | Lot 2 | 2.31 | 1.55-3.07 | |
| | Across both kit lots | 3.45 | 2.58-4.32 | |
| Influenza A | Lot 1 | 4.33 | 2.64-6.02 | CEID50/mL |
| H3N2 | Lot 2 | 3.96 | 2.64-5.27 | |
| | Across both kit lots | 4.02 | 3.08-5.19 | |
| Influenza A | Lot 1 | 0.136 | 0.095-0.177 | TCID50/mL |
| H1N1-pdm09 | Lot 2 | 0.168 | 0.112-0.225 | |
| | Across both kit lots | 0.152 | 0.118-0.186 | |
| Influenza B | Lot 1 | 0.63 | 0.36-0.91 | TCID50/mL |
| | | (0.152) | (0.085-0.220) | |
| (Victoria) | Lot 2 | 0.86 | 0.55-1.18 | (Cp/mL) |
| | | (0.207) | (0.133-0.282) | |
| | Across both kit lots | 0.77 | 0.56-0.99 | |
| | | (0.186) | (0.134-0.238) | |
| Influenza B | Lot 1 | 1.46 | 0.88-2.03 | TCID50/mL |
| (Yamagata) | | (0.499) | (0.302-0.696) | (Cp/mL) |
| | Lot 2 | 2.37 | 1.39-3.36 | |
| | | (0.806) | (0.472-1.140) | |
| | Across both kit lots | 1.98 | 1.43-2.52 | |
| | | (0.671) | (0.486-0.856) | |
| RSV A | Lot 1 | 0.030 | 0.017-0.042 | TCID50/mL |
| | Lot 2 | 0.036 | 0.020-0.051 | |
| | Across both kit lots | 0.033 | 0.023-0.043 | |
| RSV B | Lot 1 | 0.016 | 0.010-0.022 | TCID50/mL |
| | Lot 2 | 0.017 | 0.010-0.024 | |
| | Across both kit lots | 0.016 | 0.012-0.021 | |
| B. Pertussis | Lot 1 | 62.5 | 34.5-90.4 | CFU/mL |
| | Lot 2 | 59.5 | 33.0-86.0 | |
| | Across both kit lots | 61.0 | 41.7-80.3 | |
| B. Parapertussis | Lot 1 | 1.50 | 0.93-2.07 | CFU/mL |
| | Lot 2 | 1.34 | 0.77-1.91 | |
| | Across both kit lots | 1.44 | 1.04-1.84 | |
| Mycoplasma | Lot 1 | 6.35 | 3.91-8.79 | CCU/mL |
| | | (39.6) | (24.4-54.8) | |
| | Lot 2 | 3.50 | 2.12-4.89 | (Cp/mL) |
| | | (21.8) | (13.2-30.4) | |
| | Across both kit lots | 5.26 | 3.80-6.71 | |
| | | (32.7) | (23.7-41.8) | |
| Chlamydia | Lot 1 | 0.187 | 0.119-0.254 | TCID50/mL |
| | Lot 2 | 0.333 | 0.201-0.464 | |
| | Across both kit lots | 0.276 | 0.204-0.348 | |

*Less than three levels resulting in a positive hit rate between 0.1-0.9 and therefore, no statistical output is presented, see section 7.2.

### Reported False Results

Of 3690 tested cartridges, 22 provided false reported results, i.e., 0.6%. The overall false callout rate was below the criterion of 1.4%.

### External Controls

All external controls passed the acceptance criteria.

### CONCLUSION

The LODs for the Xpert Respiratory Panel target organisms were estimated by probit analysis and verified at levels where each analyte was consistently detected at a rate of ≥95% of the samples tested. The verified LOD for each Xpert Respiratory Panel target organism was lower than the values defined in Table 3, and the acceptance criteria were therefore met.

### Example 2: Crosstalk

Crosstalk is the undesirable optical signal present in a channel that is due to and directly proportional to the optical signal in an adjacent channel. Crosstalk can be either positive or negative, and crosstalk from different neighboring channels is additive. Preliminary crosstalk factors were determined for both amplification and melt curves (data not shown).

A reduced crosstalk study was performed to obtain preliminary crosstalk factors for both amplification and melt curves. A small melt crosstalk was observed from TxR to Alx532 (data not shown). Otherwise, only melt crosstalk to non-melt channels was detected. Thus, no melt crosstalk factors were set at this stage, since the result in non-melt channels does not affect the result output, and the melt channel is separated in TxR & Alx532 and thus should not cause a false result.

The final primary crosstalk factors for both amplification and melt curves were evaluated. In addition, preliminary residual crosstalk factors were also defined.

### Example 3: Respiratory Panel Assessment of Cut-Off

### Purpose

To assess the change of the assay definition file (ADF) settings, and its impact on the analytical, clinical and manufacturing specifications.

Initial results from the LOD study indicated that the Respiratory Panel assay could report positive results at the high Ct/low titer range. The late positive call outs origin from sub-LOD levels of pathogens, not detected by the comparator assay, contamination from the environment or sporadic unspecific amplifications.

The changes described below was implemented to ensure manufacturability by increased robustness to contamination from the environment during manufacturing and handling of assay cartridge. The changes were also implemented to further decrease the risk of false positive call outs due to potential unforeseen oligonucleotide interactions or other unspecific amplifications. In addition, optical settings were adjusted to decrease the risk of unnecessary invalid call outs due to curve fit misalignments and crosstalk.

### Supporting Data

To assess possible settings changes, data from pre-clinical study II, preliminary kit stability, development lot data and analytical studies were reviewed for each target, where applicable. Settings changes aimed to increase the robustness against positives call out due to environmental contamination and unspecific amplification without having significant impact on clinical performance, kit shelf life or analytical sensitivity. Setting changes also aimed to decrease unnecessary invalid call outs.

### SARS-COV-2 Change of Valid Max Cycle

In the pre-clinical II study a total of 8 samples were detected SARS-COV-2 positive with Respiratory Panel while reported negative with the comparator assay hence assigned as false positives. In data from design verification studies (collected up to the point of the design change) a total of 4 replicates with unexpected SARS-COV-2 positive call outs have been observed.

By decreasing the max valid cycle from 40 to 39, the number of false positives from the design verification studies would be reduced by 3. One additional sample is assessed as false negative in the pre-clinical study.

The preliminary kit stability data with the latest technical feasibility lot was reviewed. The new valid max cycle would cause three replicates to be reported as negative for cartridges stored in 50° C. For cartridges stored at 4° C., 28° C. and 35° C. all tested time point has Ct-values well below the new valid max cycle with no change to reported result.

PQC data for SARS-COV-2 from development lots are presented in FIG. 8. The new valid max cycle would not change any reported result for SARS-Cov-2 control material and the mean Ct would not be affected.

### Influenza A (Flu A) Change of Valid Max Cycle

In the pre-clinical II study one sample was reported Flu A positive with Respiratory Panel while reported negative with the comparator assay hence assigned as false positives. In data from design verification studies (collected up to the point of the design change), one replicate with unexpected Flu A positive call outs have been observed. This replicate had a Ct-value/EPF of 41.7/70.

By decreasing the valid max cycle from 45 to 40 for Flu A both mentioned samples with a false positive call out will be reported Flu A negative. The decreased max valid cycle would not generate any false negative results in the pre-clinical II study since all true positive samples have Ct below 40.

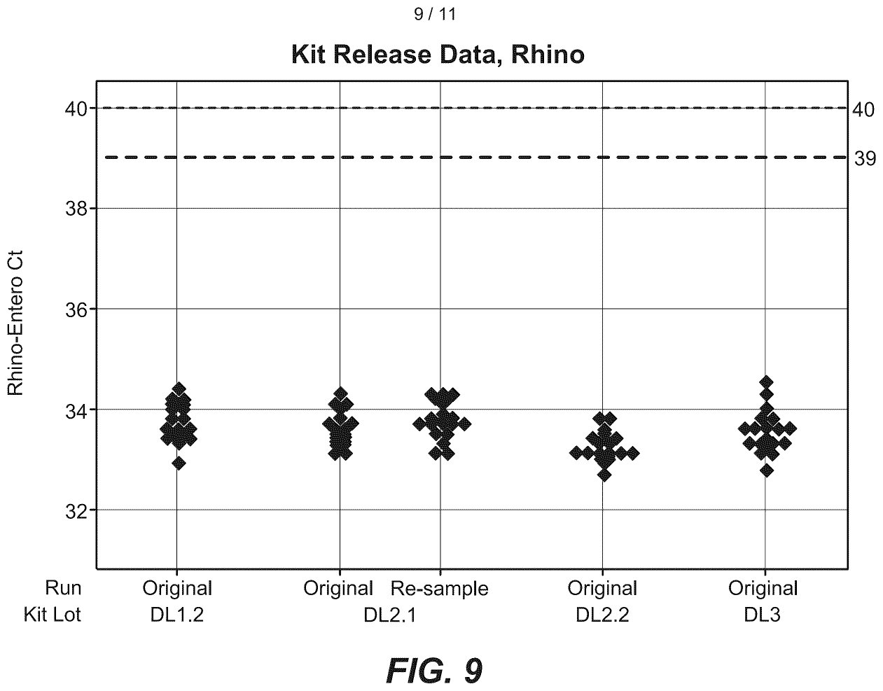

PQC data for FluA and FluA H1N1-09 from development lots are presented in FIG. 9. The new valid max cycle would not change any reported result for neither the Flu A control material nor the Flu A H1N1-09, and the mean Ct would not be affected.

### RSV Change of Manual Threshold Fluorescence Unit

In the pre-clinical II study a total of 3 samples (2 clinical samples and 1 contrived) were detected RSV positive with Respiratory Panel while reported negative with the comparator assay hence assigned as false positives. During design verification, a total of 6 replicates have given unexpected RSV positive call outs. By increasing threshold to 50, 1 false positive from the pre-clinical II study (clinical samples) and 5 replicates with unexpected positive results from design verification would be reported RSV negative.

Increasing the manual threshold fluorescence would result in a Ct-shift. The preliminary kit stability data was re-analyzed with the increased threshold and shifted Cts. Thresholds of 35 and 50 were evaluated and compared with the current threshold of 20. Shifted Ct-values with evaluated thresholds would not change any reported results in the preliminary kit stability study. Time point zero (T=0) would get a Ct at approximately 33-34 with threshold 50.

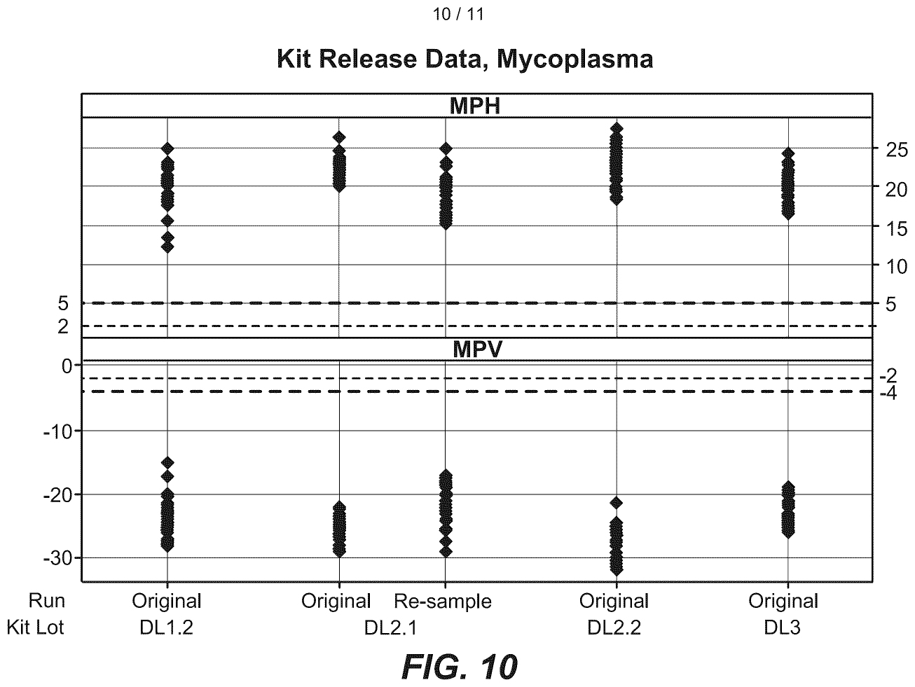

PQC data for RSV from development lots are presented in FIG. 10. The new manual threshold would not change any reported result for neither the RSV-A control material nor the RSV-B.

### Rhino-Enterovirus Change of Valid Max Cycle, Melt Peak Threshold, Melt Valley Threshold, and Melt Window

The Rhino-Enterovirus target is detected by amplification and/or melt. By decreasing the max valid cycle from 40 to 39 and by adjusting melt setting (melt peak threshold from 2 to 6 and melt peak valley from −2 to −4) several false positive call outs obtained during design verification would be reported Rhino-Enterovirus negative.

In preliminary kit stability studies, one Rhino strain have been included which is detected primarily by amplification. By decreasing the max valid cycle from 40 to 39 one outlier replicate at time point zero (T=0) would have a changed reported result from positive to negative. All other data points are below the new valid max cycle and would therefor remain as positive callouts.

Enterovirus is primarily detected by melt curves. From preliminary inclusivity studies Coxsackievirus have been identified to have weaker melt curves compared to other Enterovirus strains. To evaluate the effect of new settings, three Coxsackievirus strains were diluted to levels close to LOD and the data was analyzed with the new melt peak and melt valley thresholds. Coxsackie strain A21 and A9 are the required inclusivity strains and they would be reported as positive at LOD level with updated melt settings. Coxsackie strain B5 (not a required inclusivity strain) might have slightly higher LOD with the updated settings.

By decreasing the max temperature for the melt window from 75° C. to 65° C., the risk to get false call outs due to erroneous melt curves would be reduced. A Rhino-Enterovirus Ct detection is expected in addition to the melt curve for all melt curves with a Tₘ above 65° C., hence a positive call out will be generated based on amplification.

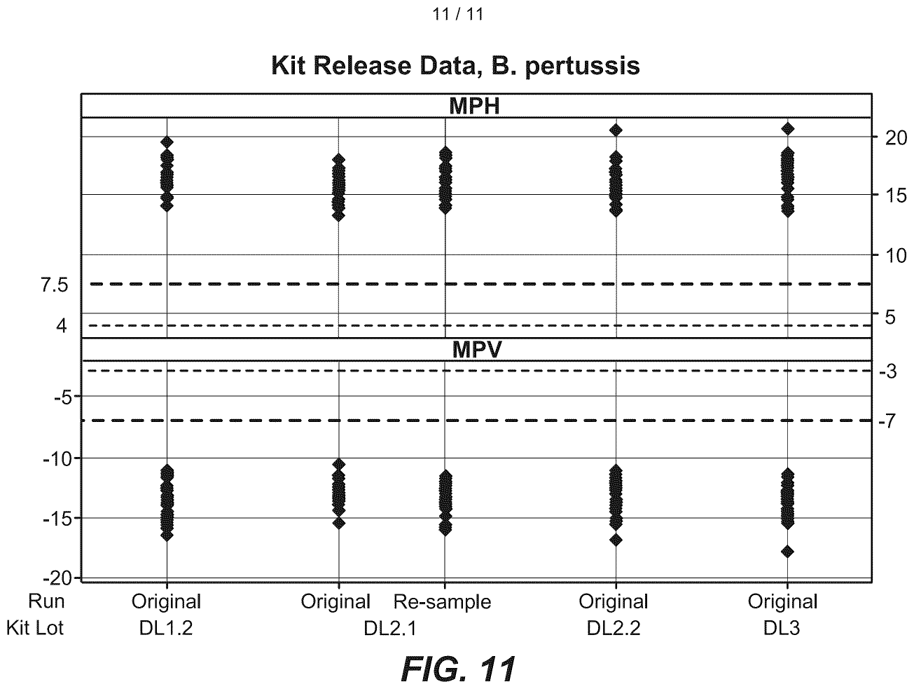

Similarly, as in the preliminary kit stability studies one Rhino strain is included in the testing. PQC data for Rhino from development lots are presented in FIG. 11. The new valid max cycle would not change any reported result for the Rhinovirus control material and the mean Ct would not be affected. The melt settings will not affect the data since the Rhinovirus control material is detected by amplification.

Mycoplasma (Myco. pneumoniae) Change of Melt Peak Threshold and Melt Valley Threshold

Background noise close to melt peak height and valley thresholds have been observed for Myco. pneumoniae. To reduce the risk of false positive call out due to background noise the thresholds are increased. In the pre-clinical II no samples were assessed as false positive or false negative compared to comparator. By increasing the melt peak threshold from 2 to 5 and decreasing the melt valley threshold from −2 to −4 no samples would have changed reported result. All Myco. pneumoniae positive sample would still be reported as positive.

In data from design verification studies (collected up to the point of the design change) a total of 6 replicates with unexpected Myco. pneumoniae positive call outs have been observed. By adjusting threshold of melt peaks (melt peak threshold from 2 to 5 and melt peak valley from −2 to −4), 2 of these will be reported Myco. pneumoniae negative.

PQC data for Myco from development lots are presented in FIG. 12. The new melt peak thresholds would not change any reported result for the Myco control material and the melt peak height and valley values would not be affected.

Bordetella pertussis (B. pertussis) Melt Peak Threshold and Melt Valley Threshold

A total of 12 samples were detected as B. pertussis false positives compared to Biofire in the pre-clinical II study. In data from design verification studies (collected up to the point of the design change) a total of 3 replicates with unexpected B. pertussis positive call outs have been observed.

By adjusting melt setting (melt peak threshold from 4 to 7.5 and melt peak valley from −3 to −7), the number of false positives from the pre-clinical II study would be reduced by 4 and from the design verification studies reduced by 2. One additional sample would be assessed as false negative in the pre-clinical II study.

PQC data for B. pertussis from development lots are presented in FIG. 13. The new melt peak thresholds would not change any reported result for the B. pertussis control material and the melt peak height and valley values would not be affected.

### Coronavirus and Adenovirus Curve Fit

Crosstalk from FAM-2, where the SPC is detected, to CF7 (Adenovirus) and CF9 (Coronavirus) has during the LOD study been observed to cause miss-shaped curve for true positive samples with low levels of Adenovirus and Coronavirus. The miss-shaped curves are failing the curve fit settings (shape and NRSE criteria) and is reported as invalid runs. The likelihood of having these crosstalk issues is assessed as high since the FAM-2 (SPC) will always have a positive call with the potential to affect the result from CF7 and CF9. To reduce the risk of getting shape or NRSE errors for samples at LOD levels the curve fit setting was changed for Adenovirus and Coronavirus.

### CF3 (Alx532) to FAM-2 Crosstalk Factor

The crosstalk study performed during technical feasibility concluded on a crosstalk of an average of −0.3% from CF3 to FAM-2. According to the Procedure for Measuring Assay-Specific Crosstalk Correction Factors, values of crosstalk <0.5% may be rounded to zero, which was done in the original crosstalk factor settings.

A follow-up study was performed during design and development showing similar crosstalk. However, since CF3 generally have strong signals even a low percent crosstalk has a noticeable effect in the FAM-2 channel with the risk of generating background errors. To reduce this risk, a crosstalk factor of −0.5% was added from CF3 to FAM. The added negative crosstalk factor entails no risk of generating false positive in FAM-2 since this channel detects the SPC, which always should be positive.

### Residual Crosstalk

The effect of residual crosstalk factors has been evaluated on all data generated data up to this point. Residual crosstalk factors doing false corrections or with no evidence of doing necessary correction will be removed. Removing non-contributing factors reduces the risk of false corrections, which might otherwise result in incorrect results.

### Impact of the Change

Impact on Analytical Performance: Data generated up to this point has been reanalyzed and the analytical LODs is determined based on updated setting. Re-analysis of data is deemed sufficient since updates to ADF settings only impacts post-run analysis of data. Hence, no studies need to be repeated.

Based on the changed reported results, new PPA and NPA have been calculated for all targets. Table 5 reflects the performance in the pre-clinical II study prior and post the change.

TABLE 5
Pre-clinical II PPA and NPA prior and post change

| | PPA | | NPA | | Target |
|---|---|---|---|---|---|
| | Prior | Post | Prior | Post | Post |
| Target | Change | Change | Change | Change | Change |
| Adenovirus | 90.2% | 92.7% | 99.3% | 99.3% | MET |
| Coronavirus | 97.3% | 97.3% | 99.8% | 99.7% | MET |
| SARS-CoV-2 | 97.9% | 97.4% | 99.3% | 99.3% | MET |
| Metapneumovirus | 95.0% | 95.0% | 100% | 100% | MET |
| Rhinovirus/Enterovirus | 97.1% | 93.8% | 96.5% | 98.1% | MET |
| Parainfluenza virus | 94.0% | 94.0% | 99.4% | 99.4% | MET |
| Influenza A virus | 100% | 100% | 99.9% | 100% | MET |
| Influenza A H1N1 2009 | 100% | 100% | 100% | 100% | MET |
| virus | | | | | |
| Influenza B virus | 100% | 100% | 100% | 100% | MET |
| Respiratory syncytial | 96.4% | 96.4% | 99.7% | 99.8% | MET |
| virus | | | | | |
| Bordetella pertussis | 100% | 96.3% | 98.8% | 99.2% | MET |
| Bordetella parapertussis | 100% | 100% | 100% | 100% | MET |
| Mycoplasma pneumoniae | 100% | 100% | 100% | 100% | MET |
| Chlamydia pneumoniae | 100% | 100% | 100% | 100% | MET |

1. A cartridge for detecting and/or identifying viral and bacterial respiratory pathogens in a sample, the cartridge comprising:
a cartridge body comprising a plurality of chambers therein, wherein the plurality of chambers includes:
a sample chamber having at least a fluid outlet in fluid communication with another chamber of the plurality; and
an optional lysis chamber in fluidic communication with the sample chamber, optionally wherein the sample chamber and lysis chamber are the same;
a reaction vessel fluidically coupled to the plurality of chambers of the cartridge body and configured for: i) amplification of nucleic acid and ii) detection and identification of a plurality of amplification products via real-time PCR, melt curve analysis, or a combination thereof;
a filter disposed in a fluidic path between the lysis chamber, if present, or the sample chamber, and the reaction vessel, and
a set of primers and optional probes for detecting and/or identifying the presence of α-coronavirus, β-coronavirus, SARS-COV-2, adenovirus, Chlamydia pneumoniae, Influenza A, Influenza B, metapneumovirus, rhinovirus/enterovirus, Mycoplasma, Bordetella spp., parainfluenza, and respiratory syncytial virus (RSV).

2. (canceled)

3. The cartridge of claim 1, wherein the reaction vessel comprises up to four reaction chambers for detection of the plurality of amplification products in solution.

4. The cartridge of claim 1, wherein the reaction vessel comprises one reaction chamber for multiplex amplification and detection of the plurality of amplification products in solution.

5. The cartridge of claim 1, wherein detection and identification of the plurality of amplification products is via real-time PCR and melt curve analysis.

6. The cartridge of claim 3, wherein each reaction chamber is configured to simultaneously amplify and detect at least 10 amplification products via real-time PCR.

7. The cartridge of claim 3, wherein each reaction chamber is configured to simultaneously detect up to 10 amplification products via melt curve analysis.

8. A method for detecting and/or identifying viral and bacterial respiratory pathogens in a sample, the method comprising:
a) contacting nucleic acid from the sample with a set of primers and optional probes for detecting the presence of α-coronavirus, β-coronavirus, SARS-COV-2, adenovirus, Chlamydia pneumoniae, Influenza A, Influenza B, metapneumovirus, rhinovirus/enterovirus, Mycoplasma, Bordetella spp., parainfluenza, and respiratory syncytial virus (RSV);
b) subjecting the nucleic acid, primers, and optional probes to amplification conditions followed by a melt curve assay;
c) detecting the presence of any amplification product(s) via real-time PCR and melt curve analysis; and
d) differentially identifying the presence of a viral and/or bacterial respiratory pathogen in the sample, or determining that no viral or bacterial pathogen detectable using the set of primers is present, based on detection of the amplification product(s) or lack thereof, respectively.

9. The method of claim 8, wherein the method further comprises administering a treatment regimen to a subject based on detecting the presence of at least one of the viral or bacterial respiratory pathogens and/or the severity of an infection.

10. (canceled)

11. The method of claim 8, wherein the bacterial respiratory pathogens are identified via melt curve analysis and the viral respiratory pathogens identified via real-time PCR and/or melt curve analysis.

12. (canceled)

13. The method of claim 8, wherein:
a) said contacting nucleic acid from the sample with the set of primers and optional probes comprises:
placing the sample in a cartridge comprising a cartridge body having a plurality of chambers in fluidic communication, a reaction vessel having one or more reaction chambers and configured for amplification and detection of the nucleic acid, a fluidic path between the plurality of chambers and the reaction vessel, and a filter in the fluidic path; and
if the sample comprises cells, lysing cells in the sample with one or more lysis reagents present within at least one of the plurality of chambers;
b) said subjecting the nucleic acid, primer pairs, and optional probes to amplification conditions comprises amplifying the nucleic acid with primers and probes present in solution within the reaction vessel.

14. The method of claim 13, wherein said detecting is carried out in up to four reaction chambers in solution.

15. The method of claim 13, wherein said detecting is carried out in a single reaction chamber in solution.

16.-18. (canceled)

19. The cartridge of claim 1, wherein:
the primers and optional probe(s) for detecting α-coronavirus are capable of hybridizing to the spike glycoprotein gene of α-coronavirus;
the primers and optional probe(s) for detecting β-coronavirus are capable of hybridizing to the ORF1a gene of β-coronavirus A;
the primers and optional probe(s) for detecting SARS-COV-2 coronavirus are capable of hybridizing to the E gene, N gene, RDRP gene, or a combination thereof, of SARS-COV-2 coronavirus;
the primers and optional probe(s) for detecting adenovirus are capable of hybridizing to the hexon protein gene of Adenovirus A, B, C, D, and E;
the primers and optional probe(s) for detecting Chlamydia are capable of hybridizing to the ompA gene of Chlamydia pneumoniae;
the primers and optional probe(s) for detecting Influenza A are capable of hybridizing to the HA gene, the MP gene, the PA gene, the PB gene, or a combination thereof, of Influenza A;
the primers and optional probe(s) for detecting Influenza B are capable of hybridizing to the MP gene, NS gene, or a combination thereof of Influenza B;
the primers and optional probe(s) for detecting metapneumovirus are capable of hybridizing to the N gene of human metapneumovirus;
the primers and optional probe(s) for detecting rhinovirus/enterovirus are capable of hybridizing to 5′ UTR gene of rhinovirus/enterovirus;
the primers and optional probe(s) for detecting Mycoplasma are capable of hybridizing to the P1 adhesion protein gene of Mycoplasma;
the primers and optional probe(s) for detecting Bordetella comprise primers and optional probe(s) that are capable of hybridizing to the IS1001 gene of Bordetella parapertussis;
the primers and optional probe(s) for detecting Bordetella also comprise primers and optional probe(s) that are capable of hybridizing to the IS481 gene of Bordetella pertussis;
the primers and optional probe(s) for detecting parainfluenza are capable of hybridizing to the polymerase (L) protein gene, nucleocapsid (NP) protein gene, or a combination thereof, of parainfluenza 1, 2, 3, and 4; and
the primers and optional probe(s) for detecting RSV are capable of hybridizing to the RSV A gene, the RSV B gene, or a combination thereof.

20. The cartridge of claim 1, wherein, when present:
the primers and optional probe(s) capable of selectively hybridizing to the ORF1a gene of CoV-OC43 comprise a sequence that is identical or complementary to at least 15 contiguous nucleotides of SEQ ID NO:24;
the primers and optional probe(s) capable of selectively hybridizing to the ORF1a gene of CoV-HKU1 comprise a sequence that is identical or complementary to at least 15 contiguous nucleotides of SEQ ID NO:28;
the primers and optional probe(s) capable of selectively hybridizing to the S gene of CoV-229E comprise a sequence that is identical or complementary to at least 15 contiguous nucleotides of SEQ ID NO:20;
the primers and optional probe(s) capable of selectively hybridizing to the S gene of CoV-NL63 comprise a sequence that is identical or complementary to at least 15 contiguous nucleotides of SEQ ID NO:32;
the primers and optional probe(s) capable of selectively hybridizing to the N gene of SARS-COV-2 comprise a sequence that is identical or complementary to at least 15 contiguous nucleotides of SEQ ID NO:96;
the primers and optional probe(s) capable of selectively hybridizing to the RDRP gene of SARS-COV-2 comprise a sequence that is identical or complementary to at least 15 contiguous nucleotides of SEQ ID NO:104;
the primers and optional probe(s) capable of selectively hybridizing to the E gene of SARS-COV-2 comprise a sequence that is identical or complementary to at least 15 contiguous nucleotides of SEQ ID NO:100;
the primers and optional probe(s) capable of selectively hybridizing to the hexon protein gene of Adenovirus A, B, C, D, and E comprise a sequence that is identical or complementary to at least 15 contiguous nucleotides of SEQ ID NO:4;
the primers and optional probe(s) capable of selectively hybridizing to the ompA gene of Chlamydia pneumoniae comprise a sequence that is identical or complementary to at least 15 contiguous nucleotides of SEQ ID NO:16;
the primers and optional probe(s) capable of selectively hybridizing to the PB gene of Influenza A comprise a sequence that is identical or complementary to at least 15 contiguous nucleotides of SEQ ID NO:40;
the primers and optional probe(s) capable of selectively hybridizing to the MP gene of Influenza A comprise a sequence that is identical or complementary to at least 15 contiguous nucleotides of SEQ ID NO:36;
the primers and optional probe(s) capable of selectively hybridizing to the PA gene of Influenza A comprise a sequence that is identical or complementary to at least 15 contiguous nucleotides of SEQ ID NO:44;
the primers and optional probe(s) capable of selectively hybridizing to the H1 gene of Influenza A comprise a sequence that is identical or complementary to at least 15 contiguous nucleotides of SEQ ID NO:48;
the primers and optional probe(s) capable of selectively hybridizing to the MP gene of Influenza B comprise a sequence that is identical or complementary to at least 15 contiguous nucleotides of SEQ ID NO:52,
the primers and optional probe(s) capable of selectively hybridizing to the NS gene of Influenza B comprise a sequence that is identical or complementary to at least 15 contiguous nucleotides of one or more of SEQ ID NO:56;
the primers and optional probe(s) capable of selectively hybridizing to the N gene of human metapneumovirus comprise a sequence that is identical or complementary to at least 15 contiguous nucleotides of SEQ ID NO:60;
the primers and optional probe(s) capable of selectively hybridizing to 5′ UTR gene of rhinovirus/enterovirus comprise a sequence that is identical or complementary to at least 15 contiguous nucleotides of SEQ ID NO:84;
the primers and optional probe(s) capable of selectively hybridizing to the P1 adhesion protein gene of Mycoplasma pneumoniae comprise a sequence that is identical or complementary to at least 15 contiguous nucleotides of SEQ ID NO:64;
the primers and optional probe(s) capable of selectively hybridizing to the IS1001 gene of Bordetella parapertussis comprise a sequence that is identical or complementary to at least 15 contiguous nucleotides of SEQ ID NO:8;
the primers and optional probe(s) capable of selectively hybridizing to the IS481 gene of Bordetella pertussis comprise a sequence that is identical or complementary to at least 15 contiguous nucleotides of SEQ ID NO: 12;
the primers and optional probe(s) capable of selectively hybridizing to the polymerase (L) protein gene of Parainfluenza 1 and 3 comprise a sequence that is identical or complementary to at least 15 contiguous nucleotides of one or more of SEQ ID NOs: 68 and 76, respectively;
the primers and optional probe(s) capable of selectively hybridizing to the nucleocapsid (NP) protein gene of Parainfluenza 2 and 4 comprise a sequence that is identical or complementary to at least 15 contiguous nucleotides of one or more of SEQ ID NO: 72 and 80, respectively;
the primers and optional probe(s) capable of selectively hybridizing to the RSV A gene comprise a sequence that is identical or complementary to at least 15 contiguous nucleotides of SEQ ID NO:88; and
the primers and optional probe(s) capable of selectively hybridizing to the RSV B gene comprise a sequence that is identical or complementary to at least 15 contiguous nucleotides of SEQ ID NO:92.

21.-22. (canceled)

23. The cartridge of claim 1, wherein the cartridge or kit further comprises, or the method further employs, a primer pair specific for an exogenous control and/or an endogenous control, wherein the exogenous control is a sample processing control, and wherein the endogenous control is a sample adequacy control.

24. (canceled)

25. The cartridge of claim 2, wherein the one or more lysis reagents provide simultaneous lysis of viral and bacterial cells.

26. (canceled)

27. A system for detecting pathogens in a biological sample, the system comprising:
a module having a receiving bay for receiving the cartridge of claim 1, wherein the module includes one or more mechanisms within the receiving bay for manipulating a fluid sample within the cartridge, and an instrument that interfaces with the reaction vessel; and
a memory having programmable instructions recorded thereon, that are specially configured to operate the module according to a respiratory pathogen assay protocol to determine nucleic acid sequence characteristics of α-coronavirus, β-coronavirus, SARS-COV-2, adenovirus, Chlamydia pneumoniae, Influenza A, Influenza B, metapneumovirus, rhinovirus/enterovirus, Mycoplasma, Bordetella spp., parainfluenza, and respiratory syncytial virus (RSV).

28. A method for identifying up to forty target nucleic acids in a biological sample collected from a subject, the method comprising:
a. providing a sample cartridge comprising:
i. a plurality of chambers including: a lysis chamber having at least a fluid outlet in fluid communication with another chamber of the plurality, the lysis chamber adapted for performing mechanical and chemical lysis to release nucleic acid from the biological sample;
ii. lyophilized and liquid reagents in two or more of the plurality of chambers, the lyophilized reagents comprising at least one primer set for amplifying and detecting the target nucleic acid(s), and the liquid reagent including a lysis buffer, a binding reagent, and an eluting buffer; and
iii. a reaction vessel fluidically coupled to the plurality of chambers of the sample cartridge and configured for performing amplification of the target nucleic acids;
b. contacting nucleic acid from the biological sample with the at least one primer set,
i. wherein each primer set comprises a forward primer, a reverse primer, and a probe specific for a target nucleic acid in the biological sample, and
ii. wherein at least one of the forward primer or the reverse primer within each set comprises one or more modified bases selected from a destabilizing base, a stabilizing base, or a combination thereof; and
iii. wherein the probe in each set comprises a detectable moiety for detection via real-time PCR or melt curve analysis;
c. subjecting the nucleic acid, primers, and probes within the reaction vessel to amplification conditions, optionally followed by a melt curve assay,
d. detecting the presence of any amplification product(s) via real-time PCR and optionally melt curve analysis, by an optical assembly, wherein the optical assembly comprises at least ten optical channels configured to simultaneously transmit excitation beams in at least ten excitation wavelength ranges, and simultaneously detect emitted light in at least ten emission wavelength ranges to and from the reaction vessel, respectively; and
e. differentially identifying the presence of the target nucleic acid(s) in the sample, or determining that no target nucleic acid is detectable based on detection of the amplification product(s) or lack thereof, respectively,
wherein when greater than 10 target nucleic acids are to be identified, one or more probes for detection via melt curve analysis and one or more probes for detection via real-time PCR are combined in a single optical channel.

29.-40. (canceled)

41. A sample cartridge for identifying up to forty target nucleic acids in a biological sample collected from a subject, the cartridge comprising:
a. a cartridge body comprising a plurality of chambers therein, wherein the plurality of chambers includes:
i. a sample chamber having at least a fluid outlet in fluid communication with another chamber of the plurality;
ii. a lysis chamber in fluidic communication with the sample chamber, wherein the lysis chamber is adapted for performing mechanical and chemical lysis to release nucleic acid from the biological sample, optionally wherein the sample chamber and lysis chamber are the same;
i. a lyophilized reagent chamber comprising a lyophilized PCR master mix including at least one primer set for amplifying and detecting the target nucleic acids, further
a. wherein each primer set comprises a forward primer, a reverse primer, and a probe specific for a target nucleic acid in the biological sample, and
b. wherein at least one of the forward primer or the reverse primer within each set comprises one or more modified bases selected from a destabilizing base, a stabilizing base, or a combination thereof; and
c. wherein the probe in each set comprises a detectable moiety for detection via real-time PCR or melt curve analysis;
iii. one or more liquid reagent chambers comprising a lysis buffer, a binding reagent, and an eluting buffer;
b. a reaction vessel fluidically coupled to the plurality of chambers of the cartridge body and configured for amplification of nucleic acid and melt curve assay, and adapted for detection of one or a plurality of amplification products via real-time PCR and melt curve analysis by an optical assembly,
wherein the optical assembly comprises at least ten optical channels configured to simultaneously transmit excitation beams in at least ten excitation wavelength ranges to the reaction vessel, and simultaneously detect emitted light in at least ten emission wavelength ranges from the reaction vessel; and
c. a filter disposed in a fluidic path between the lysis chamber, and the reaction vessel; and
wherein when greater than 10 target nucleic acids are to be identified, one or more probes for detection via melt curve analysis and one or more probes for detection via real-time PCR are combined in a single optical channel.

42. A diagnostic assay system for identifying up to forty target nucleic acids in a biological sample collected from a subject, the system comprising:
a. a sample cartridge according to claim 1;
b. a thermal assembly configured to subject the nucleic acid, primers, and probes within the reaction vessel of the sample chamber to amplification conditions, optionally followed by a melt curve assay;
c. an optical assembly comprising at least ten optical channels and configured to simultaneously transmit excitation beams in at least ten excitation wavelength ranges, and simultaneously detect emitted light in at least ten emission wavelength ranges to and from the reaction vessel, respectively;
d. an electronic device for
i. receiving and processing signals detected due to the presence of any amplification product(s) via real-time PCR and melt curve assay, by the optical assembly;
ii. differentially identifying the presence of a target nucleic acid in the sample, or determining that no target nucleic acid is detectable based on detection of the amplification product(s), melt curve, or lack thereof; and
iii. outputting a real-time PCR result for the sample in the reaction vessel during or after the amplification, and outputting a melting result for the sample in the reaction vessel during or after the melt probe assay,
e. a communication interface in communication with an electronic device that comprises a display screen having a user interface that displays one or more graphical elements that permit a user to input a selection corresponding to at least one target assay from a plurality of target assays, which at least one target assay is directed to determining presence, absence or amount of at least one target nucleic acid in the biological sample.


SEQUENCE LISTING (104 sequences)

SEQ ID NO: 1 - DNA, 23 bp, synthetic construct
  source 1..23: mol_type = other DNA
gccacggtggggtttctaaactt

SEQ ID NO: 2 - DNA, 25 bp, synthetic construct
  source 1..25: mol_type = other DNA
tgatgccgcagtgggcatacatgca

SEQ ID NO: 3 - DNA, 44 bp, synthetic construct
  source 1..44: mol_type = other DNA
cccccccctcggtgcaccagacccgggctcaggtactccgaggc

SEQ ID NO: 4 - DNA, 136 bp, synthetic construct
  source 1..136: mol_type = other DNA
gccacggtggggtttctaaacttgtttcccagggtgaagtaggtgtccgtggcgcgggcgaattgcaccagaccgggactcaggtactccgaggcatcctgaccggcgatgtgcatgtaagaccactgcggcatca

SEQ ID NO: 5 - DNA, 16 bp, synthetic construct
  source 1..16: mol_type = other DNA
ccggctcgacgaattg

SEQ ID NO: 6 - DNA, 21 bp, synthetic construct
  source 1..21: mol_type = other DNA
ccagagccgtttgagttcgtc

SEQ ID NO: 7 - DNA, 41 bp, synthetic construct
  source 1..41: mol_type = other DNA
ggcgccaaccagccgctgctgacggtctatgtcctggcgcc

SEQ ID NO: 8 - DNA, 79 bp, synthetic construct
  source 1..79: mol_type = other DNA
ccggctcgacgaattgctgcaagccaaccagccgctgctgacggtctatgtcctgcgtgacgaactcaaacggctctgg

SEQ ID NO: 9 - DNA, 25 bp, synthetic construct
  source 1..25: mol_type = other DNA
cacacaaacttgatgggcgatcaat

SEQ ID NO: 10 - DNA, 23 bp, synthetic construct
  source 1..23: mol_type = other DNA
agcttggtcggatgaacacccat

SEQ ID NO: 11 - DNA, 43 bp, synthetic construct
  source 1..43: mol_type = other DNA
ccccccggcgggttcgagtcgacgtaggaaggtcaatcccgcc

SEQ ID NO: 12 - DNA, 94 bp, synthetic construct
  source 1..94: mol_type = other DNA
cacacaaacttgatgggcgatcaattgctggaccatttcgagtcgacgtaggaaggtcaatcgggcatgcttatgggtgttcatccggccgggc

SEQ ID NO: 13 - DNA, 21 bp, synthetic construct
  source 1..21: mol_type = other DNA
gcgttgctttccccttgtcaa

SEQ ID NO: 14 - DNA, 24 bp, synthetic construct
  source 1..24: mol_type = other DNA
gatagagaggctcctacttgccat

SEQ ID NO: 15 - DNA, 45 bp, synthetic construct
  source 1..45: mol_type = other DNA
ccccccagcctgcagactttgttctagtagctgttgttacaggct

SEQ ID NO: 16 - DNA, 103 bp, synthetic construct
  source 1..103: mol_type = other DNA
gcgttgctttccccttgccaacagacgctggcgtagcaacagctactggaacaaagtctgcgaccatcaattatcatgaatggcaagtaggagcctctctatc

SEQ ID NO: 17 - DNA, 25 bp, synthetic construct
  source 1..25: mol_type = other DNA
tactggtgttaatgatgctattaca

SEQ ID NO: 18 - DNA, 24 bp, synthetic construct
  source 1..24: mol_type = other DNA
ttgattaacaacatcctggatctt

SEQ ID NO: 19 - DNA, 29 bp, synthetic construct
  source 1..29: mol_type = other DNA
cttcacaagctctacaaacagttgctact

SEQ ID NO: 20 - DNA, 91 bp, synthetic construct
  source 1..91: mol_type = other DNA
tactggtgttaatgatgctattacacaaacttcacaagctctacaaacagttgctactgcacttaataagatccaggatgttgttaatcaa

SEQ ID NO: 21 - DNA, 28 bp, synthetic construct
  source 1..28: mol_type = other DNA
tttaatcgtatagctagttgtaaatgga

SEQ ID NO: 22 - DNA, 21 bp, synthetic construct
  source 1..21: mol_type = other DNA
ccttttgcgtttctgcagcaa

SEQ ID NO: 23 - DNA, 25 bp, synthetic construct
  source 1..25: mol_type = other DNA
agctaatgaatgtacagagcgcttg

SEQ ID NO: 24 - DNA, 103 bp, synthetic construct
  source 1..103: mol_type = other DNA
tttaatcgtatagctagttgtaaatggaccgatgtggatgattacatactagctaatgaatgtacagagcgcttgaaattgtttgctgcagaaacgcaaaagg

SEQ ID NO: 25 - DNA, 28 bp, synthetic construct
  source 1..28: mol_type = other DNA
tttaataagatagctagttgtaaatgga

SEQ ID NO: 26 - DNA, 21 bp, synthetic construct
  source 1..21: mol_type = other DNA
ccttttgagtttctgcagcaa

SEQ ID NO: 27 - DNA, 27 bp, synthetic construct
  source 1..27: mol_type = other DNA
ctggcaaatgagtgtattgaacgttta

SEQ ID NO: 28 - DNA, 103 bp, synthetic construct
  source 1..103: mol_type = other DNA
tttaataagatagctagttgtaaatggacagaagttgatgattatgttctggcaaatgagtgtattgaacgtttaaagttatttgctgcagaaactcaaaagg

SEQ ID NO: 29 - DNA, 25 bp, synthetic construct
  source 1..25: mol_type = other DNA
tagtagcgttaatgatgctattaca

SEQ ID NO: 30 - DNA, 24 bp, synthetic construct
  source 1..24: mol_type = other DNA
ttgattaacaacatcctgaatctt

SEQ ID NO: 31 - DNA, 31 bp, synthetic construct
  source 1..31: mol_type = other DNA
ctgcagaggctatacatactgttactattgc

SEQ ID NO: 32 - DNA, 91 bp, synthetic construct
  source 1..91: mol_type = other DNA
tagtagcgttaatgatgctattacacaaactgcagaggctatacatactgttactattgcacttaataagattcaggatgttgttaatcaa

SEQ ID NO: 33 - DNA, 20 bp, synthetic construct
  source 1..20: mol_type = other DNA
ttctaaccgaggtcgaaacg

SEQ ID NO: 34 - DNA, 19 bp, synthetic construct
  source 1..19: mol_type = other DNA
attggtcttgtctttagcc

SEQ ID NO: 35 - DNA, 20 bp, synthetic construct
  source 1..20: mol_type = other DNA
tcaggccccctcaaagccga

SEQ ID NO: 36 - DNA, 145 bp, synthetic construct
  source 1..145: mol_type = other DNA
ttctaaccgaggtcgaaacgtacgttctttctatcatcccgtcaggccccctcaaagccgagatcgcacagagactggaaagtgtctttgcaggaaagaacacagatcttgaggctcttatggaatggctaaagacaagaccaat

SEQ ID NO: 37 - DNA, 20 bp, synthetic construct
  source 1..20: mol_type = other DNA
aaacgggactctagcatact

SEQ ID NO: 38 - DNA, 20 bp, synthetic construct
  source 1..20: mol_type = other DNA
taattgatggccatccgaat

SEQ ID NO: 39 - DNA, 22 bp, synthetic construct
  source 1..22: mol_type = other DNA
ctgacagccagacagcgaccaa

SEQ ID NO: 40 - DNA, 68 bp, synthetic construct
  source 1..68: mol_type = other DNA
aaacgggactctagcatacttactgacagccagacagcgaccaaaagaattcggatggccatcaatta

SEQ ID NO: 41 - DNA, 25 bp, synthetic construct
  source 1..25: mol_type = other DNA
atcttggggggctatatgaagcaat

SEQ ID NO: 42 - DNA, 19 bp, synthetic construct
  source 1..19: mol_type = other DNA
aggaaggagttgaaccaag

SEQ ID NO: 43 - DNA, 20 bp, synthetic construct
  source 1..20: mol_type = other DNA
aatgatccctgggttttgct

SEQ ID NO: 44 - DNA, 88 bp, synthetic construct
  source 1..88: mol_type = other DNA
atcttggggggctatatgaagcaatcgaggagtgcttgattaatgatccctgggttttgcttaatgcatcttggttcaactccttcct

SEQ ID NO: 45 - DNA, 24 bp, synthetic construct
  source 1..24: mol_type = other DNA
cctgggaaatccagagtgtgaatc

SEQ ID NO: 46 - DNA, 24 bp, synthetic construct
  source 1..24: mol_type = other DNA
atctcctgggtaacacgttccatt

SEQ ID NO: 47 - DNA, 44 bp, synthetic construct
  source 1..44: mol_type = other DNA
cggcgtagatgtttccgcaatgtagggccatgagcttgacgccg

SEQ ID NO: 48 - DNA, 100 bp, synthetic construct
  source 1..100: mol_type = other DNA
cctgggaaatccagagtgtgaatcactctccacagcaagctcatggtcctacattgtggaaacatctagttcagacaatggaacgtgttacccaggagat

SEQ ID NO: 49 - DNA, 19 bp, synthetic construct
  source 1..19: mol_type = other DNA
ttggagacacgattgccta

SEQ ID NO: 50 - DNA, 19 bp, synthetic construct
  source 1..19: mol_type = other DNA
aggtcaaattctttcccac

SEQ ID NO: 51 - DNA, 19 bp, synthetic construct
  source 1..19: mol_type = other DNA
ctgctagttctgctttgcc

SEQ ID NO: 52 - DNA, 106 bp, synthetic construct
  source 1..106: mol_type = other DNA
ttggagacacgattgcctacctgctttcattaacagaagatggagaaggcaaagcagaactagcagaaaaattacactgttggttcggtgggaaagaatttgacct

SEQ ID NO: 53 - DNA, 19 bp, synthetic construct
  source 1..19: mol_type = other DNA
gatggccatcggatcctca

SEQ ID NO: 54 - DNA, 20 bp, synthetic construct
  source 1..20: mol_type = other DNA
gctcttgaccaaattgggat

SEQ ID NO: 55 - DNA, 21 bp, synthetic construct
  source 1..21: mol_type = other DNA
  modified_base 11: mod_base = OTHER; note = Uracil
caaagccaattcgagcagctg

SEQ ID NO: 56 - DNA, 112 bp, synthetic construct
  source 1..112: mol_type = other DNA
gatggccatcggatcctcaattcactcttcgagcgtcttaatgaaggacattcaaagccaattcgagcagctgaaactgcggtgggagtcttatcccaatttggtcaagagc

SEQ ID NO: 57 - DNA, 29 bp, synthetic construct
  source 1..29: mol_type = other DNA
catacaagcatgctatattaaaagagtct

SEQ ID NO: 58 - DNA, 25 bp, synthetic construct
  source 1..25: mol_type = other DNA
cctatttctgcagcatatttgtaat

SEQ ID NO: 59 - DNA, 24 bp, synthetic construct
  source 1..24: mol_type = other DNA
ttcttgttgcaatgatgaaggtgt

SEQ ID NO: 60 - DNA, 163 bp, synthetic construct
  source 1..163: mol_type = other DNA
catataaacatgctatattaaaagagtctcaatacacaataaaaagagatgtaggcaccacaactgcagtgacaccttcatcattgcagcaagagataacacttttgtgtggagagattctttacactaaacatactgattacaaatatgctgcagagatagg

SEQ ID NO: 61 - DNA, 22 bp, synthetic construct
  source 1..22: mol_type = other DNA
cgcaccctactacttccacaat

SEQ ID NO: 62 - DNA, 23 bp, synthetic construct
  source 1..23: mol_type = other DNA
atccttaccgttcttgtcctctc

SEQ ID NO: 63 - DNA, 23 bp, synthetic construct
  source 1..23: mol_type = other DNA
ataaaggtatcaactacagtttg

SEQ ID NO: 64 - DNA, 103 bp, synthetic construct
  source 1..103: mol_type = other DNA
cgcaccctactacttccacaataaccccgattgacaagaccgtccaatccaaaatgtggttgatgcctttattaagccctgagaggacaagaacggtaaggat

SEQ ID NO: 65 - DNA, 22 bp, synthetic construct
  source 1..22: mol_type = other DNA
cataatccaagaggaggaatag

SEQ ID NO: 66 - DNA, 29 bp, synthetic construct
  source 1..29: mol_type = other DNA
ctatggcttgattgtctccttgaaccatt

SEQ ID NO: 67 - DNA, 31 bp, synthetic construct
  source 1..31: mol_type = other DNA
cagctgcaagatggattgcactaatagagat

SEQ ID NO: 68 - DNA, 136 bp, synthetic construct
  source 1..136: mol_type = other DNA
cataatccaagaggaggaatagaaggttattgccagaaattatggacactaatctctattagtgcaatccatcttgcagctgttaaagttggtgtcagagtgtcagcaatggtacaaggagacaatcaagctatag

SEQ ID NO: 69 - DNA, 22 bp, synthetic construct
  source 1..22: mol_type = other DNA
taggacccatggccaagtacat

SEQ ID NO: 70 - DNA, 23 bp, synthetic construct
  source 1..23: mol_type = other DNA
gacagttccaatacccattgcat

SEQ ID NO: 71 - DNA, 28 bp, synthetic construct
  source 1..28: mol_type = other DNA
caccaaagctgatggattttgtcccatc

SEQ ID NO: 72 - DNA, 110 bp, synthetic construct
  source 1..110: mol_type = other DNA
taggacccatggccaagtacatggctctattagaatcaccaaagctgatggattttgtcccatctgaatatccattagtctatagttatgcaatgggtattggaactgtc

SEQ ID NO: 73 - DNA, 22 bp, synthetic construct
  source 1..22: mol_type = other DNA
cataacccaagagggggtatag

SEQ ID NO: 74 - DNA, 29 bp, synthetic construct
  source 1..29: mol_type = other DNA
ctatggcttgattgtctccttgaaccatt

SEQ ID NO: 75 - DNA, 30 bp, synthetic construct
  source 1..30: mol_type = other DNA
cagctgctagatgtattgcacttatggata

SEQ ID NO: 76 - DNA, 127 bp, synthetic construct
  source 1..127: mol_type = other DNA
cataacccaagagggggtatagaaggattttgtcaaaaattatggacactcatatctataagtgcaatacatctagcagctgttagaataggcgtgagggtgactgcaatggttcaaggagacaatc

SEQ ID NO: 77 - DNA, 26 bp, synthetic construct
  source 1..26: mol_type = other DNA
tttccttgcaggccacatcaatgcag

SEQ ID NO: 78 - DNA, 24 bp, synthetic construct
  source 1..24: mol_type = other DNA
gggtctattgcatcaacttcaatg

SEQ ID NO: 79 - DNA, 24 bp, synthetic construct
  source 1..24: mol_type = other DNA
gcatctggggctctggcagcaatc

SEQ ID NO: 80 - DNA, 97 bp, synthetic construct
  source 1..97: mol_type = other DNA
tttccttgcaggccacatcaatgcagaatcatcttatgattgctgccagagccccagatgctgctcttaggattattgaagttgatgcaatagatcc

SEQ ID NO: 81 - DNA, 18 bp, synthetic construct
  source 1..18: mol_type = other DNA
gtcctccggtccctgaat

SEQ ID NO: 82 - DNA, 19 bp, synthetic construct
  source 1..19: mol_type = other DNA
gaaacacggacacccaaag

SEQ ID NO: 83 - DNA, 39 bp, synthetic construct
  source 1..39: mol_type = other DNA
ccccccgtcgtaatgggtaattgtgggatgggaccgact

SEQ ID NO: 84 - DNA, 121 bp, synthetic construct
  source 1..121: mol_type = other DNA
gtcctccggcccctgaatgcggctaaccctaaccctgcagccattgcccacaatccagtgggtttatggtcgtaatgagcaattgcgggatgggaccgactactttgggtgtccgtgtttc

SEQ ID NO: 85 - DNA, 26 bp, synthetic construct
  source 1..26: mol_type = other DNA
tacactcaacaaagatcaacttctgt

SEQ ID NO: 86 - DNA, 27 bp, synthetic construct
  source 1..27: mol_type = other DNA
ttctgcacatcataattaggagtgtca

SEQ ID NO: 87 - DNA, 22 bp, synthetic construct
  source 1..22: mol_type = other DNA
ctcctgtgctccgttggatggt

SEQ ID NO: 88 - DNA, 96 bp, synthetic construct
  source 1..96: mol_type = other DNA
tacactcaacaaagatcaacttctgtcatccagcaaatacaccatccaacggagcacaggagatagtattgacactcccaattatgatgtgcagaa

SEQ ID NO: 89 - DNA, 24 bp, synthetic construct
  source 1..24: mol_type = other DNA
cattaaataaggatcagctgctgt

SEQ ID NO: 90 - DNA, 26 bp, synthetic construct
  source 1..26: mol_type = other DNA
tttgcacatcataattgggagtgtca

SEQ ID NO: 91 - DNA, 25 bp, synthetic construct
  source 1..25: mol_type = other DNA
tctcctgtactacgttgaatagtgt

SEQ ID NO: 92 - DNA, 93 bp, synthetic construct
  source 1..93: mol_type = other DNA
cattaaataaggatcagctgctgtcatccagcaaatacactattcaacgtagtacaggagataatattgacactcccaattatgatgtgcaaa

SEQ ID NO: 93 - DNA, 22 bp, synthetic construct
  source 1..22: mol_type = other DNA
tgattacaaacattggccgcaa

SEQ ID NO: 94 - DNA, 20 bp, synthetic construct
  source 1..20: mol_type = other DNA
aaagcgcgacattccgaaga

SEQ ID NO: 95 - DNA, 23 bp, synthetic construct
  source 1..23: mol_type = other DNA
ctgaagcgctgggggcaaattgt

SEQ ID NO: 96 - DNA, 73 bp, synthetic construct
  source 1..73: mol_type = other DNA
tgattacaaacattggccgcaaattgcacaatttgcccccagcgcttcagcgttcttcggaatgtcgcgcatt

SEQ ID NO: 97 - DNA, 26 bp, synthetic construct
  source 1..26: mol_type = other DNA
acaggtacgttaatagttaatagcgt

SEQ ID NO: 98 - DNA, 21 bp, synthetic construct
  source 1..21: mol_type = other DNA
atattgcagcagtacgcacac

SEQ ID NO: 99 - DNA, 26 bp, synthetic construct
  source 1..26: mol_type = other DNA
acactagccatccttactgcgcttcg

SEQ ID NO: 100 - DNA, 113 bp, synthetic construct
  source 1..113: mol_type = other DNA
acaggtacgttaatagttaatagcgtacttctttttcttgctttcgtggtattcttgctagttacactagccatccttactgcgcttcgattgtgtgcgtactgctgcaatat

SEQ ID NO: 101 - DNA, 22 bp, synthetic construct
  source 1..22: mol_type = other DNA
cctcacttgttcttgctcgcaa

SEQ ID NO: 102 - DNA, 24 bp, synthetic construct
  source 1..24: mol_type = other DNA
cactcaatacttgagcacactcat

SEQ ID NO: 103 - DNA, 24 bp, synthetic construct
  source 1..24: mol_type = other DNA
caacgtgttgtagcttgtcacacc

SEQ ID NO: 104 - DNA, 93 bp, synthetic construct
  source 1..93: mol_type = other DNA
cctcacttgttcttgctcgcaaacatacaacgtgttgtagcttgtcacaccgtttctatagattagctaatgagtgtgctcaagtattgagtg